SEQUENCE LISTING (14 sequences)

SEQ ID NO: 1 - protein, 1273 aa, Artificial Sequence
  Synthetic vaccine01 | L5P L7S S13N K386E D389N K417N S477N E484K S494P N501Y
MFVFPVSLPLVSNQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTELNNLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVKGFNCYFPLQPYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, Artificial Sequence
  Synthetic vaccine02 | L5P L7S S13N D80A K386E K417N S477N E484K S494P N501Y
MFVFPVSLPLVSNQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTELNDLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVKGFNCYFPLQPYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1272 aa, Artificial Sequence
  Synthetic vaccine03 | F4S L5P L7S S12P S13N V120I D215G C291-S477N
MFVSPVSLPLVPNQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIINNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHRSYLTPGDSSSGWTAGAAAYYVGYLQPRTFLLKYNENGTITDAVDALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1270 aa, Artificial Sequence
  Synthetic vaccine04 | L5R L7S S13N P26S F55L H69- Y145- S221-A372V K417N S477N N501Y
MFVFRVSLPLVSNQCVNLTTRTQLPSAYTNSFTRGVYYPDKVFRSSVLHSTQDLLLPFFSNVTWFHAIVSGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSVSFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1270 aa, Artificial Sequence
  Synthetic vaccine05 | L5P L7S S13N P26S D80A T240- L241- L242-T323I K417N R457G K458R E484K N501Y
MFVFPVSLPLVSNQCVNLTTRTQLPSAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQALHRSYLTPGDSSSGWTAGAAAYYVGYLQPRTFLLKYNENGTITDAVDCALDPLSETKCTLKSFTVEKGIYQTSNFRVQPIESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFGRSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1273 aa, Artificial Sequence
  Synthetic vaccine06 | L5P L7S S13N V327I K386E K417N S477N E484K S494P N501Y
MFVFPVSLPLVSNQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIIRFPNITNLCPFGEVFNATRFASVYAWNRKRISNCVADYSVLYNSASFSTFKCYGVSPTELNDLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVKGFNCYFPLQPYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1271 aa, Artificial Sequence
  Synthetic vaccine07 | L5P L7S S13N T240- L242- K417N R457G E484K T500I N501Y
MFVFPVSLPLVSNQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFGKSNLKPFERDISTEIYQAGSTPCNGVKGFNCYFPLQSYGFQPIYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1270 aa, Artificial Sequence
  Synthetic vaccine08 | L5R L7S S13N H69- V70- Y145- I472M E484K N501Y A570D P681H D1118H K1191N
MFVFRVSLPLVSNQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEMYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVANNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1270 aa, Artificial Sequence; identical to SEQ ID NO: 8
  Synthetic vaccine09 | L5R L7S S13N H69- V70- Y145- I472M E484K N501Y A570D P681H D1118H K1191N

SEQ ID NO: 10 - protein, 1273 aa, Artificial Sequence
  Synthetic vaccine10 | L5P L7S S13N P25S K417N S477N E484K S494P N501Y Y505H
MFVFPVSLPLVSNQCVNLTTRTQLSPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGNTPCNGVKGFNCYFPLQPYGFQPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 5 aa, Artificial Sequence
  Synthetic
ATCGL

SEQ ID NO: 12 - protein, 5 aa, Artificial Sequence
  Synthetic
NLKRV

SEQ ID NO: 13 - protein, 5 aa, Artificial Sequence
  Synthetic
  misc_feature (3)..(3): Xaa is a masked amino acid
ATXGL

SEQ ID NO: 14 - protein, 5 aa, Artificial Sequence
  Synthetic
  misc_feature (4)..(4): Xaa is a masked amino acid
NLKXV

(19) United States
(12) Patent Application Publication
(10) Pub. No.: US 2024/0321387 A1
(43) Pub. Date: Sep. 26, 2024

(54) IMMUNOGEN SELECTION

(71) Applicants: BioNTech SE, Mainz (DE); InstaDeep Ltd, London (GB)

(72) Inventors: Alexander Muik, Seeheim-Jugenheim (DE); Asaf Poran, Stoneham, MA (US); Yunpeng Liu, Cambridge, MA (US); Ugur Sahin, Mainz (DE); Karim Beguir, London (GB); Marcin Skwark, London (GB); Thomas Pierrot, Paris (FR); Yunguan Fu, London (GB)

(21) Appl. No.: 18/289,424

(22) PCT Filed: May 4, 2022
(86) PCT No.: PCT/US22/27736

§ 371 Date: Nov. 3, 2023

Related U.S. Application Data
(60) Provisional application No. 63/293,611, filed on Dec. 23, 2021.
(60) Provisional application No. 63/293,649, filed on Dec. 23, 2021.
(60) Provisional application No. 63/283,430, filed on Nov. 27, 2021.
(60) Provisional application No. 63/283,206, filed on Nov. 24, 2021.

(30) Foreign Application Priority Data
May 4, 2021 (GB) 2106376.3
May 7, 2021 (GB) 2106580.0

(51) Int. Cl. G16B 15/30; G06N 3/045; G06N 3/08; G06N 5/01; G06N 20/00; G16B 40/20

(52) U.S. Cl. CPC G16B 15/30; G06N 3/045; G06N 3/08; G06N 5/01; G06N 20/00; G16B 40/20

(57) ABSTRACT

A method is provided for identifying a number of variants of concern of a reference disease associated immunogen. The method uses a language model to perform inference on data representing each of a plurality of variants and data representing the reference immunogen. For each of the plurality of variants and the reference immunogen, a characteristic vector is derived from an output feature map of a hidden layer of the language model. For each of the plurality of variants, a measure of distance is generated for the variant that includes calculating a measure of distance between the characteristic vector of the variant and the characteristic vector of the reference immunogen. A semantic change score is calculated for each variant based on the generated measure of distance for that variant. A variant of the reference immunogen is selected based, at least in part, on the generated the semantic change scores.

### CROSS REFERENCE TO RELATED APPLICATIONS

This application claims the benefit of United Kingdom Patent Application No. GB 2106376.3 filed May 4, 2021, United Kingdom Patent Application No. GB 2106580.0 filed May 7, 2021, U.S. Provisional Application No. 63/283,206 filed Nov. 24, 2021, U.S. Provisional Application No. 63/283,430 filed Nov. 27, 2021, and U.S. Provisional Application No. 63/293,611 filed Dec. 23, 2021, and U.S. Provisional Application No. 63/293,649 filed Dec. 23, 2021, the contents of each of which are hereby incorporated herein in their entirety.

### TECHNICAL FIELD

The present disclosure relates generally to immunogen selection and immunogenic compositions. In particular, the present disclosure relates to data-driven identification and selection of immunogens of interest and rational vaccine design.

### BACKGROUND

One of the biggest challenges in healthcare today is accelerating the speed of drug development. Diseases and human response to them is an ongoing race and traditional drug discovery routes and timescales cannot cope with the pace of disease evolution or spread of pathogens in the modem interconnected world.

Immunogens targeted by the immune system while responding to a disease are often susceptible to mutations. Such mutations can lead to the phenomenon of immune escape or immune evasion whereby the mutated variant antigen is no longer recognised and countered by the immune system. Pathogens, including viruses, such as coronaviruses, can mutate leading to so-called variants. If a variant is more evolutionarily fit, for example more virulent, more infectious, or notably resistant to immune response (by antibodies or T-cells), it is more competent than its peers and spreads more readily. Identification of such variants is of paramount importance, as it allows to plan for the appropriate strategy of response.

Recent attempts to identify potentially dangerous variants of a given virus have struggled to reliably predict “dangerousness” of the viral variant, with some using trained statistical models to identify most “different” variants, assuming that these would be the ones to readily escape the immune system.

Analogous work for other transmissible diseases, such as influenza (Moncla, et al, “Influenza Evolution: New Insights into an Old Foe.”; Li et al “Selection of Antigenically Advanced Variants of Seasonal Influenza Viruses.” 2016; Fleury et al “Antigen Distortion Allows Influenza Virus to Escape Neutralization.” 1998; Schey et al “Multistate Design of Influenza Antibodies Improves Affinity and Breadth against Seasonal Viruses.” 2019) or HIV (Mascola et al “HIV-1: Nature's Master of Disguise.” 2003; McKeating et al. “Characterization of HIV-1 Neutralization Escape Mutants.” 1989; Althaus et al “Dynamics of Immune Escape during HIV/SIV Infection.” 2008; Wei et al. “Antibody Neutralization and Escape by HIV-1.” 2003; Leslie et al. “HIV Evolution: CTL Escape Mutation and Reversion after Transmission.” 2004; Burton et al. “HIV Vaccine Design and the Neutralizing Antibody Problem.” 2004; Goulder et al. “Evolution and Transmission of Stable CTL Escape Mutations in HIV Infection.” 2001). The attempts to decipher the broad, general patterns of discerning the “meaningful” variants have usually been focused on frequentist statistics (Schey et al. “Multistate Design of Influenza Antibodies Improves Affinity and Breadth against Seasonal Viruses.” 2019; Sevy et al. “Integrating Linear Optimization with Structural Modeling to Increase HIV Neutralization Breadth.” 2018; Crowe et al “Principles of Broad and Potent Antiviral Human Antibodies: Insights for Vaccine Design.” 2017).

Recently, there has been an increased interest in using technology deployed with success in Natural Language Processing for modelling causal relationships in protein sequences. Models like ProtBERT or FaceBook Research ESM-1b (Elnaggar et al. “ProtTrans: Towards Cracking the Language of Life's Code Through Self-Supervised Deep Learning and High Performance Computing.” 2020; Rives et al. “Biological Structure and Function Emerge from Scaling Unsupervised Learning to 250 Million Protein Sequences.” 2019; Rao et al. “Transformer Protein Language Models Are Unsupervised Structure Learners.” 2020) are available. However, each trained ML model is usable for what it has been trained for, and general-purpose models are not sufficiently granular for specific tasks.

There remains, therefore, a need for data driven fully-automated methods to identify variants of concern in diseases where the immunogens being targeted are susceptible to mutation.

### SUMMARY

According to a first aspect of the present invention, there is provided a method of selecting one or more variant of a reference disease associated immunogen for preparing an immunogenic composition. Another aspect provides a method of identifying a number of variants of concern of a reference disease associated immunogen. The number may be the top 10-20, such as top 10, 11 or 12, variants of concerns. The method can be used to detect and monitor high risk variants. The variants include one or more variant of the reference immunogen. In some embodiments the reference immunogen may be a wild-type immunogen, such as variants of SARS-CoV-2. These methods comprise: using a language model to perform inference on data representing each of a plurality of variants and data representing the reference immunogen; determining, for each of the plurality of variants and the reference immunogen, a characteristic vector derived from an output feature map of a hidden layer of the language model; for each of the plurality of variants, generating a measure of distance for the variant that includes calculating a measure of distance between the characteristic vector of the variant and the characteristic vector of the reference immunogen; generating a semantic change score for each variant based on the generated measure of distance for that variant; selecting a variant of the reference immunogen based, at least in part, on the generated the semantic change scores.

The language model used in the method may be one of an ensemble of language models comprising a plurality of language models, wherein the extracted characteristic vectors are derived from an output feature map of each language model in the ensemble of language models. The ensemble of language models may comprise at least one of a Recurrent neural network and Transformer-based model. Each of the language models within the ensemble may have been trained on a dataset of protein sequences. The language model may have been trained on a dataset of naturally-occurring variants of the reference immunogen. The naturally-occurring variants of the reference immunogen may be newly-sequenced variants.

The step of generating a semantic change score may comprise ranking the plurality of variants by their respective generated measures of distance and then transforming the ranked values into scaled semantic change scores.

The method may further comprise identifying one or more epitope regions of the reference immunogen and identifying each corresponding range of positions associated with each epitope in the data representing the reference immunogen. The epitope regions identified above may be assigned different weightings. The weightings may correspond to a measure of confidence of the existence of the epitope. The method may comprise generating an epitope value for each variant based on the location of mutations in that variant relative to the reference immunogen and the assigned weightings. The epitope value may vary depending on whether the one or more mutations occur on or close to positions on the variant that correspond to positions identified as being associated with the epitope of the reference immunogen. In some embodiments, the epitope value may vary depending upon a surface availability of the position of the mutation.

The method may comprise generating an epitope score for each variant from the epitope values by ranking the plurality of variants by the respective epitope value and then transforming the ranked values into a scaled epitope score.

Selecting a variant of the reference immunogen may be based, at least in part, on the generated semantic change scores and the epitope scores.

The method may comprise a step of calculating an immune escape score that is a combination of the semantic change score and the epitope score. The immune escape score may be an average of the semantic change score and the epitope score.

The method may further comprise calculating, for each variant, a likelihood value for the data representing the variant. The data representing the variant may be data describing a protein sequence of the variant. The likelihood value for the data representing the variant may be derived from a likelihood for each amino acid in the data describing the protein sequence.

The method may comprise calculating a likelihood value for each variant based, at least in part, on the likelihood for the data representing the variant. The likelihood for a variant may be calculated based on an output of inference performed by the language model on data representing the variant. The likelihood may be calculated as a log likelihood. The likelihood value may be an overall likelihood calculated for a protein sequence. The overall likelihood may be calculated from a sum of likelihoods for each amino acid in the protein sequence. In other embodiments, the likelihood value may be calculated from an average of likelihoods for each amino acid in the protein sequence.

The process for deriving the likelihood values may comprise masking at least one data point in the data representing a variant to generate a masked sequence of data, wherein the data point represents an amino acid in a protein sequence of the variant. The method may include performing inference using the language model on the masked sequence of data to identify a likelihood of the one or more amino acids corresponding to the at least one data point within the sequence of data. The method may include repeating the masking and inference until a likelihood of each amino acid within the protein sequence has been identified. The method may include summing the likelihoods across the amino acids to calculate a likelihood value of the variant. A likelihood score for the variant may be generated based, at least in part, on the likelihood value of the variant.

In a case that the language model is one of an ensemble of language models comprising a plurality of language models, the likelihood value for a variant may be derived from a likelihood generated by each of the language models in the ensemble. The likelihood value of a variant may be an average of the likelihoods generated by the language models in the ensemble.

The method may comprise generating a likelihood score for each variant from the likelihood values by ranking the plurality of variants by their respective likelihood value. The method may comprise transforming the ranking into a scaled likelihood score using a linear projection.

The method may comprise determining a binding value that represents an estimated binding energy between a variant and a corresponding human receptor. Determining the binding value may include simulating one or more structures associated with the variant. The structure may correspond to a model of a folded protein sequence of the variant using the data representing the variant. Determining the binding value may further comprise estimating, using the simulated one or more structures, a change of binding energy when an interface of the structure is bound with a model of a human receptor.

Simulating the structure of a particular variant may comprise modifying a known three-dimensional structure of a related variant to have the same protein sequence as the particular variant and selecting a plurality of conformational configurations of the resulting structure of the particular variant in order to identify one or more structures with a minimum energy.

The method of determining the binding energy for a particular variant may comprise estimating a binding energy of a simulated structure of a particular variant with a human receptor. The method of determining the binding energy for a particular variant may comprise estimating a size of an interface area between the structure of a particular variant and a human receptor. The binding value may be the binding energy, the estimated size of the interface area, or a combination (such as a ratio) of the binding energy and estimated size of the interface area.

In order to reduce computational burden, the simulation of the structure of each variant may be performed only for a part of the data representing the variant that is known to form the binding interface with the human receptor.

The method may comprise generating a binding score for each variant from the binding values by ranking the plurality of variants by their respective binding value. The method may comprise transforming the rankings into a scaled binding score.

The method may comprise determining a growth value for each variant. The growth value may be a measure of growth of submissions associated with each variant within a dataset.

The method may comprise generating a growth score for each variant from the growth values by ranking the plurality of variants by their respective growth value. The method may comprise transforming the rankings into a scaled growth score.

Selecting a variant of the reference immunogen may be based, at least in part, on one or more of the likelihood score, the binding score, and the growth score.

The method may comprise a step of calculating an infectivity score that is a combination of the likelihood score, the binding score, and the growth score. The immune escape score may be an average of the likelihood score, the binding score, and the growth score.

The method may further comprise calculating a Pareto score which is determined by calculating a Pareto front using the immune escape score and the infectivity score. Calculating the Pareto score may comprise calculating a first Pareto front corresponding to a set of variants for which there does not exist any other variant with both higher immune escape and infectivity score. Calculating the Pareto score may further comprise calculating successive Pareto fronts from the set of variants remaining after removing the first or succeeding Pareto fronts. The method may comprise calculating successive Pareto fronts until all variants are assigned to a front. Each variant is assigned a Pareto value depending on the number of the front that they were member of. The Pareto score may then be obtained by transforming the Pareto values to a scaled Pareto score.

In the method, generating a score for each variant may be based, at least in part, on additional scores provided by trained human experts.

The data representing each of the plurality of variants may comprise data representing a protein sequence of the variant and the data representing the reference immunogen may comprise data representing a protein sequence of the reference immunogen. The data representing protein sequences of the respective variants and the reference immunogen may comprise a plurality of data points representing a plurality of amino acids in a plurality of positions within each protein sequence.

The characteristic vector for each variant may be derived from a plurality of vectors from output feature maps generated by the language model, each output feature map being associated with an amino acid in the data representing the variant. The characteristic vector may be derived from an average of the plurality of vectors from the output feature maps generated by the language model in association with different amino acids.

Where the language model is a Recurrent neural network, the characteristic vector may be derived from a final embedding layer of the Recurrent neural network. Where the language model is a Transformer-based language model, the characteristic vector may be derived from a final transformer layer of the Transformer-based language model.

The characteristic vector for a variant may be a concatenation of a plurality of characteristic vectors for that variant, each of the plurality of characteristic vectors for that variant being derived from a hidden layer of a separate language model of the ensemble of language models.

The measures of distance of each variant from the reference immunogen may be L1 distances. The reference immunogen may be a wild-type immunogen. The step of generating a measure of distance for the variant may comprise calculating a measure of distance between the characteristic vector of the variant and the characteristic vector of the reference immunogen and calculating a measure of distance between the characteristic vector of the variant and the characteristic of one or more additional reference immunogens. In one example, in the case of SARS-CoV-2 spike protein, the reference immunogen may be the variant sequenced in Wuhan and the one or more additional reference immunogens may be the D614G variant. The measure of distance may be a sum of the Euclidean distances of the characteristic vectors. In embodiments in which the variants are ranked by measure of distance and a semantic change score is calculated the result of use of one or more additional reference immunogens to generate the measure of distance does not affect the scaling of the resulting semantic change score.

The method according to preceding aspects may comprise a step of training the language model. The training of the language model may be performed by self-supervised learning. Training the language model may include masking one or more amino acids of a protein sequence from a training dataset to form one or more masked protein sequences. The language model may perform inference based on the one or more masked protein sequences. A loss function may be defined based on a difference between the results of inference and the one or more protein sequences from the training dataset. In some embodiments, a probabilistic masking scheme may be used in the training of the language model.

The method may be performed by an information processing apparatus. The information processing apparatus may comprise hardware accelerators, such as graphics processing units or neural processing units.

Methods according to the first aspect may act as an early warning system for detection of SARS-CoV-2.

According to a second aspect of the present invention there is provided a method for use in association with therapeutic or vaccine development, the method comprising: using a language model to perform inference on data representing each of a plurality of variants and data representing a reference disease associated immunogen; determining, for each of the plurality of variants and the reference immunogen, a characteristic vector derived from an output feature map of a hidden layer of the language model; for each of the plurality of variants, generating a measure of distance for the variant that includes calculating a measure of distance between the characteristic vector of the variant and the characteristic vector of the reference immunogen; generating a semantic change score for each variant based on the generated measure of distance for that variant; selecting a variant of the reference immunogen based, at least in part, on the generated the semantic change scores.

According to a further aspect of the present disclosure, there is provided an immunogenic composition comprising one or more immunogens selected or identified using the methods as per any element of the previous aspects.

Another aspect provides a method of making an immunogenic composition comprising selecting one or more immunogens for inclusion in the composition as per the method of any preceding aspect. The method of making the composition may further include formulation the selected immunogens with adjuvants and/or carriers for administration to subjects.

Another aspect provides an immunogenic composition for use in a method of vaccinating or treating a subject, the vaccination or treatment method comprising selecting one or more immunogens using the methods as per any element of the previous aspects. The vaccination or treatment methods may also include formulation of the selected immunogens with adjuvants and/or carriers for administration to subjects. Also provided are methods for treating or vaccinating a subject by administering an immunogenic composition comprising one or more variant immunogens selected or identified using the methods as per any element of the previous aspects.

The treatment or vaccination is in respect of a disease associated with the reference immunogen. The disease may be an infectious disease caused by a pathogen such as virus, bacteria or other parasites.

According to a yet further aspect of the present disclosure, there is provided a method of performing a trend analysis on the prevalence of variants of concern of a reference immunogen in a subject or a population. The analysis may be a weekly identification of a number of variants of concern. The number of variants of concern may be the top 10-20, such as top 10, 11 or 12, variants of concerns out of the known variants being analysed by the method. The method can be used to monitor high risk variants. The variants include one or more variant of the reference immunogen. In some embodiments the reference immunogen is a wild-type immunogen, such as variants of SARS-CoV-2. These methods comprise: using a language model to perform inference on data representing each of a plurality of variants and data representing the reference immunogen; determining, for each of the plurality of variants and the reference immunogen, a characteristic vector derived from an output feature map of a hidden layer of the language model; for each of the plurality of variants, generating a measure of distance for the variant that includes calculating a measure of distance between the characteristic vector of the variant and the characteristic vector of the reference immunogen; generating a semantic change score for each variant based on the generated measure of distance for that variant; selecting a variant of the reference immunogen based, at least in part, on the generated the semantic change scores.

Methods according to this aspect may act as an early warning system for detection of SARS-CoV-2.

In one example of all aspects, the pathogen is SARS-CoV-2 and the disease is COVID-19. The reference immunogen in this case may be SARS-CoV-2, the spike protein or SARS-CoV-2 or one or more epitopes of the spike protein of SARS-CoV-2.

In one example of all aspects, the pathogen is the influenza virus and the disease is influenza. The reference immunogen in this case may be a virus subtype, such as H5N1, or a surface protein from the virus such as hemagglutinin or neuraminidase.

The subject may be human or a non-human animal.

According to further aspects of the invention, there is provided a method of selecting immunogens for inclusion in an immunogenic composition by identifying at least one variant of a reference immunogen, the method comprising: using a language model to perform inference on data representing each of a plurality of variants and data representing the reference immunogen; extracting, for each of the plurality of variants, a characteristic vector derived from an output feature map of a hidden layer of the language model; performing a dimension reduction on each characteristic vector to generate a plurality of points in a shared lower dimensional space, each point being associated with a different variant or the reference immunogen; identifying clusters of variants in the shared lower dimensional space and calculating a middle point for each cluster; generating at least one distance score for each variant, wherein the distance score is derived at least in part from a measure of distance calculated between the characteristic vector corresponding to that variant and the middle point of at least one cluster of variants; and identifying at least one variant based on the at least one distance score and selecting at least one immunogen from that at least one variant for inclusion in the immunogenic composition.

In some embodiments, the reference immunogen is a wild-type immunogen.

Identifying a variant based on the distance score may comprise: splitting the plurality of variants into groups based on one or more behaviour descriptors; within each group of variants, establishing a Pareto front with respect to a plurality of characteristics of the variants, wherein establishing the Pareto front comprises retaining in the group only variants forming the relevant Pareto front and wherein a characteristic of the plurality of characteristics is the distance score; sampling the retained variants and copying the nucleic acid sequence of each sampled variant; introducing mutations into the nucleic acid sequence of each sampled variant to obtain mutated variants; determining, for each mutated variant, values for the plurality of characteristics and the at least one behaviour descriptor, the characteristics including the distance calculated between the characteristic vector corresponding to that variant and the middle point of at least one cluster of variants; selecting, for each mutated variant, a group based on the behaviour descriptors for the mutated variant, and determining, for each mutated variant, whether to add the mutated variant to the Pareto front in the selected group by comparing the plurality of characteristics of the mutated variant against the corresponding plurality of characteristics of immunogens in the Pareto front established in the selected group.

Generating at least one distance score for each variant may comprise generating a plurality of distance scores for each variant. Each of the plurality of distance scores for each variant may be derived at least in part from a measure of distance calculated between the characteristic vector corresponding to that variant and the middle point of a respective different cluster of a plurality of clusters of variants in the shared lower dimensional space.

The plurality of characteristics of each of the variants may comprise a likelihood of the variant, wherein the likelihood of the variant is determined by the language model performing inference on a protein sequence of the variant.

Each mutated variant may be added to the Pareto front if the variant is determined to be more likely than other variants in the group. The plurality of characteristics of each of the variants may comprise each of the plurality of distance scores for the variant, each distance score being a separate characteristic of the plurality of characteristics. Each mutated variant may be added to the Pareto front if the mutated variant is determined to be closer to the middle of at least one cluster than other variants in the group.

The measure of distance may be an L1 distance, and the one or more behaviour descriptors may be dimensions in the shared lower dimensional space. The method may further comprise establishing, across all the groups of variants, an overall Pareto front with respect to the plurality of characteristics.

Introducing mutations to the nucleic acid sequence of each sampled variant may be performed by changing, adding or removing one or more nucleotides in the nucleic acid sequence. The changes to the nucleic acid sequence may be performed within a set of constraints. The set of constraints may comprise at least one of: maintaining a ratio or range of ratios of transition-type mutations to transversion-type mutations; limiting mutations to predetermined portions of the sequence; and limiting mutations such that two mutations may not occur on neighbouring positions in the sequence.

The method may further comprise ranking the variants in the overall Pareto front to identify immunogens for inclusion in an immunogenic composition. The immunogenic composition may be a polyvalent composition including several immunogens, such as 2, 3 or 4 immunogens. A plurality of variants may be identified based on the at least one distance score, and a plurality of immunogens may be selected from the at least one variant for inclusion in the immunogenic composition.

According to another aspect of the present invention there is provided a method of making an immunogenic composition, the method comprising: selecting immunogens for inclusion in the immunogenic composition by identifying at least one variant of a reference immunogen, the selecting comprising: using a language model to perform inference on data representing each of a plurality of variants and data representing the reference immunogen; extracting, for each of the plurality of variants, a characteristic vector derived from an output feature map of a hidden layer of the language model; performing a dimension reduction on each characteristic vector to generate a plurality of points in a shared lower dimensional space, each point being associated with a different variant or the reference immunogen; identifying clusters of variants in the shared lower dimensional space and calculating a middle point for each cluster; generating at least one distance score for each variant, wherein the distance score is derived at least in part from a measure of distance calculated between the characteristic vector corresponding to that variant and the middle point of at least one cluster of variants; and identifying at least one variant based on the at least one distance score and selecting at least one immunogen from that variant for inclusion in the immunogenic composition. The method of such another aspect may further comprise formulating the selected immunogens with adjuvants and/or carriers for administration to subjects.

In some embodiments, the reference immunogen is a wild-type immunogen.

According to a further aspect of the present invention, there is provided an immunogenic composition for use in a method of vaccinating or treating a subject, the vaccination or treatment method comprising selecting immunogens for inclusion in the immunogenic composition by identifying at least one variant of a reference immunogen, the selecting comprising: using a language model to perform inference on data representing each of a plurality of variants and data representing the reference immunogen; extracting, for each of the plurality of variants, a characteristic vector derived from an output feature map of a hidden layer of the language model; performing a dimension reduction on each characteristic vector to generate a plurality of points in a shared lower dimensional space, each point being associated with a different variant or the reference immunogen; identifying clusters of variants in the shared lower dimensional space and calculating a middle point for each cluster; generating at least one distance score for each variant, wherein the distance score is derived at least in part from a measure of distance calculated between the characteristic vector corresponding to that variant and the middle point of at least one cluster of variants; and identifying at least one variant based on the at least one distance score and selecting at least one immunogen from that variant for inclusion in the immunogenic composition.

In some embodiments, the reference immunogen is a wild-type immunogen.

The vaccination or treatment methods may also include formulating the selected immunogens with adjuvants and/or carriers for administration to subjects.

According to a further aspect of the present invention, there is provided a method or use according to any previous aspect, wherein the immunogen is the spike protein or SARSCoV-2 and the selected immunogen for inclusion in the immunogenic composition is a sequence comprising one or more or SEQ ID Nos. 1 to 10. In one example, the selected immunogen for inclusion in the immunogenic composition comprises SEQ ID NO. 1. In one example, the selected immunogen for inclusion in the immunogenic composition comprises SEQ ID NO. 2. In one example, the selected immunogen for inclusion in the immunogenic composition comprises SEQ ID NO. 3. In one example, the selected immunogen for inclusion in the immunogenic composition comprises SEQ ID NO. 4. In one example, the selected immunogen for inclusion in the immunogenic composition comprises SEQ ID NO. 5. In one example, the selected immunogen for inclusion in the immunogenic composition comprises SEQ ID NO. 6. In one example, the selected immunogen for inclusion in the immunogenic composition comprises SEQ ID NO. 7. In one example, the selected immunogen for inclusion in the immunogenic composition comprises SEQ ID NO. 8. In one example, the selected immunogen for inclusion in the immunogenic composition comprises SEQ ID NO. 9. In one example, the selected immunogen for inclusion in the immunogenic composition comprises SEQ ID NO. 10.

According to a further aspect of the present invention there is provided a method of selecting immunogens for inclusion in an immunogenic composition by identifying at least one variant of a reference immunogen, the method comprising: using a language model to perform inference on data representing each of a plurality of variants and data representing the reference immunogen; extracting, for each of the plurality of variants, a characteristic vector derived from an output feature map of a hidden layer of the language model; identifying clusters of variants based on the characteristic vector extracted for each of the plurality of variants and calculating a middle point for each cluster; generating at least one distance score for each variant, wherein the distance score is derived at least in part from a measure of distance calculated between the characteristic vector corresponding to that variant and the middle point of at least one cluster of variants; and identifying at least one variant based on the at least one distance score and selecting at least one immunogen from that at least one variant for inclusion in the immunogenic composition.

In some embodiments, the reference immunogen is a wild-type immunogen.

Identifying a variant based on the distance score may comprise: splitting the plurality of variants into groups based on one or more behaviour descriptors; within each group of variants, establishing a Pareto front with respect to a plurality of characteristics of the variants, wherein establishing the Pareto front comprises retaining in the group only variants forming the relevant Pareto front and wherein a characteristic of the plurality of characteristics is the distance score; sampling the retained variants and copying the nucleic acid sequence of each sampled variant; introducing mutations into the nucleic acid sequence of each sampled variant to obtain mutated variants; determining, for each mutated variant, values for the plurality of characteristics and the at least one behaviour descriptor, the characteristics including the distance calculated between the characteristic vector corresponding to that variant and the middle point of at least one cluster of variants; selecting, for each mutated variant, a group based on the behaviour descriptors for the mutated variant, and determining, for each mutated variant, whether to add the mutated variant to the Pareto front in the selected group by comparing the plurality of characteristics of the mutated variant against the corresponding plurality of characteristics of immunogens in the Pareto front established in the selected group.

Generating at least one distance score for each variant may comprise generating a plurality of distance scores for each variant. Each of the plurality of distance scores for each variant may be derived at least in part from a measure of distance calculated between the characteristic vector corresponding to that variant and the middle point of a respective different cluster of a plurality of clusters of variants.

The plurality of characteristics of each of the variants may comprise a likelihood of the variant, wherein the likelihood of the variant is determined by the language model performing inference on a protein sequence of the variant.

Each mutated variant may be added to the Pareto front if the variant is determined to be more likely than other variants in the group. The plurality of characteristics of each of the variants may comprise each of the plurality of distance scores for the variant, each distance score being a separate characteristic of the plurality of characteristics. Each mutated variant may be added to the Pareto front if the mutated variant is determined to be closer to the middle of at least one cluster than other variants in the group.

The measure of distance may be an L1 distance, and the one or more behaviour descriptors may be dimensions. The method may further comprise establishing, across all the groups of variants, an overall Pareto front with respect to the plurality of characteristics.

Introducing mutations to the nucleic acid sequence of each sampled variant may be performed by changing, adding or removing one or more nucleotides in the nucleic acid sequence. The changes to the nucleic acid sequence may be performed within a set of constraints. The set of constraints may comprise at least one of: maintaining a ratio or range of ratios of transition-type mutations to transversion-type mutations; limiting mutations to predetermined portions of the sequence; and limiting mutations such that two mutations may not occur on neighbouring positions in the sequence.

The method may further comprise ranking the variants in the overall Pareto front to identify immunogens for inclusion in an immunogenic composition. The immunogenic composition may be a polyvalent composition. A plurality of variants may be identified based on the at least one distance score, and a plurality of immunogens may be selected from the at least one variant for inclusion in the immunogenic composition.

According to another aspect of the present invention there is provided a method of making an immunogenic composition, the method comprising: selecting immunogens for inclusion in the immunogenic composition by identifying at least one variant of a reference immunogen, the selecting comprising: using a language model to perform inference on data representing each of a plurality of variants and data representing the reference immunogen; extracting, for each of the plurality of variants, a characteristic vector derived from an output feature map of a hidden layer of the language model; identifying clusters of variants based on the characteristic vector extracted for each of the plurality of variants and calculating a middle point for each cluster; generating at least one distance score for each variant, wherein the distance score is derived at least in part from a measure of distance calculated between the characteristic vector corresponding to that variant and the middle point of at least one cluster of variants; and identifying at least one variant based on the at least one distance score and selecting at least one immunogen from that variant for inclusion in the immunogenic composition. The method of the eighth aspect may further comprise formulating the selected immunogens with adjuvants and/or carriers for administration to subjects.

In some embodiments the reference immunogen is a wild-type immunogen.

According to a further aspect of the present invention, there is provided an immunogenic composition for use in a method of vaccinating or treating a subject, the vaccination or treatment method comprising selecting immunogens for inclusion in the immunogenic composition by identifying at least one variant of a reference immunogen, the selecting comprising: using a language model to perform inference on data representing each of a plurality of variants and data representing the reference immunogen; extracting, for each of the plurality of variants, a characteristic vector derived from an output feature map of a hidden layer of the language model; identifying clusters of variants based on the characteristic vector extracted for each of the plurality of variants and calculating a middle point for each cluster; generating at least one distance score for each variant, wherein the distance score is derived at least in part from a measure of distance calculated between the characteristic vector corresponding to that variant and the middle point of at least one cluster of variants; and identifying at least one variant based on the at least one distance score and selecting at least one immunogen from that variant for inclusion in the immunogenic composition.

In some embodiments, the reference immunogen is a wild-type immunogen.

The vaccination or treatment methods may also include formulating the selected immunogens with adjuvants and/or carriers for administration to subjects.

According to a further of the present invention, there is provided a method or use according to any of the preceding three aspects, wherein the immunogen is the spike protein or SARS-CoV-2 and the selected immunogen for inclusion in the immunogenic composition is a sequence comprising one or more or SEQ ID Nos. 1 to 10.

Further features and advantages of the invention will become apparent from the following description of preferred embodiments of the invention, given by way of example only, which is made with reference to the accompanying drawings.

### BRIEF DESCRIPTION OF THE DRAWINGS

FIG. 1 is a schematic diagram showing an example of a process for training a deep language model.

FIG. 2 is a schematic diagram showing a fine-tuning process of training a language model.

FIG. 3 is a schematic diagram showing an example of the scoring process for a variant.

FIG. 4 is T-SNE plot before clustering is performed.

FIG. 5 is a T-SNE plot following a clustering operation.

FIG. 6A is a schematic diagram showing an example of a scoring process in a further embodiment.

FIG. 6B is a schematic diagram showing an example of the process of designing a vaccine based on naturally-occurring and de novo variants of a virus.

FIGS. 7A-7G are graphs relating to Example 1a, demonstrating the ranking of variants of concern according to the first embodiment.

FIGS. 8A-8F are graphs relating to Example 1b, demonstrating the ranking of variants of concern according to a further embodiment, wherein additional scores based on epitopes are made use of FIG. 9A is a graph showing a distribution of mutations that affect known epitopes in each of a set of selected vaccine candidates according to a vaccine selection method, labelled ‘Vaccine’, and in a sampled GISAID dataset, labelled ‘GISAID’.

FIG. 9B is a chart showing a number of different epitopes affected in each of the Vaccine and GISAID data sets.

FIG. 9C is a chart showing a number of Variant of Concern mutations in each of the Vaccine and GISAID data sets.

FIG. 9D is a violin plot diagram showing distributions of vaccine candidates having mutations within particular epitopes within the Vaccine and GISAID data sets.

FIGS. 10A to 10D are charts showing properties of protein sequences found in the GISAID data set and properties of sequences generated according to an example method.

FIG. 11 is a chart showing pareto scores of protein sequences found in the GISAID data set and pareto scores of sequences generated according to an example method.

Fig. A1. is a schematic of an Early Warning System (EWS) described in connection with Example 2. The EWS embodies structural modelling methods and natural language processing techniques to enable risk level estimation of SARS-CoV-2 variants in real-time. (A) Structural modelling is used to predict the binding affinity of SARS-CoV-2 Spike protein to host ACE2, and to score the mutated epitope regarding its impact on immune escape. (B) Machine Learning modelling (e.g. performed via machine learning model) is used to extract implicit information from unlabelled data for the hundreds of thousands of registered variants in the GISAID database. (C) EWS relies on the information from A and B to compute an immune escape score and a fitness prior (e.g. infectivity) score, which taken together, present a more comprehensive view of the SARS-CoV-2 variant landscape. Both scores can be combined to obtain a single score, based on the notion of Pareto optimality and dubbed Pareto score, that represents a variant's risk. The higher the Pareto score, the fewer variants with higher immune escape and fitness prior scores. (D) Schematic of AI model structure for assessing semantic change and log-likelihood. Once trained (see Fig. S1.A below), the model receives as input a variant spike protein sequence, and returns an embedding vector of the spike protein sequence as well as probabilities over amino-acids for each residue (see Fig. S1.B below). The embedding vector is used to calculate semantic change from the Wuhan and D614G variants while the probabilities are used to compute the log-likelihood.

Fig. A2 is a series of figures showing how in silico predicted scores for immune escape and fitness prior (e.g. infectivity) correlate with in vitro data. (A) The surface of a SARS-CoV-2 Spike protein in ‘one RBD up’ conformation (PDB id: 7kdl), in top colored by the frequency of contact of surface residues with neutralizing antibodies (brighter, warmer colors corresponds to more antibody binding). Middle and bottom row depict the number of evaded epitopes in a Beta (B.1.351) and Omicron (B.1.1.529). Left column: side view. Right column: top view. (B) Validation of the immune escape metric with pseudovirus neutralization test (pVNT) results. Relationships of the epitope score, semantic change score, and combined immune escape score with the observed 50% pseudovirus neutralization titer (pVNT₅₀) reduction are shown across n=19 selected SARS-CoV-2 variants of interest including Omicron (B.1.1.529). Cross-neutralization of n=12-40 BNT162b2-immune sera was assessed against vesicular stomatitis virus (VSV)-SARS-CoV-2-S pseudoviruses. pVNT₅₀ reduction compared to wild-type SARS-CoV-2 (Wuhan strain) Spike pseudotyped VSV is given in percent. Variants for which experimentally measured geometric mean pVNT₅₀ increased compared to the Wuhan strain have been assigned a pVNT₅₀ reduction of 0 (equal to wild type). Epitope score (based on structural modelling) indicates the number of known neutralizing antibodies (max. n=310) whose binding epitope is affected by the SARS-CoV-2 variants' mutations. Semantic change score (based on machine learning) indicates the predicted variation in the biological function between a variant and wild-type SARS-CoV-2. For the semantic change score, the distance in embedding space between the sequence in question and a reference (WT+D614G Spike protein) is compared. Sequences have then been ranked with respect to this distance and the resultant rank has been scaled in the range of [0, 100]. The immune escape score is calculated as the average of the scaled epitope score and the scaled semantic change score. Dashed lines represent the linear regression. (C and D) Validation of a component of the fitness prior (e.g. infectivity) metric, capturing the ACE2 binding propensity. Relationship of the ACE2 binding score with the experimentally determined ACE-2 binding affinity (KD, dissociation constant) are shown across n=19 RBD variants, along with a linear regression dash line. The ACE2 binding score is ranked and scaled analogously to fitness prior components, such that variants with the lowest energy are assigned a score of 100, highest −0. (D) Relationship of the ACE2 binding score with the experimentally determined ACE2 association rate (Kon) are shown across RBD variants, along with a regression dash line. The ACE2 binding score is ranked and scaled analogously to fitness prior components, such that variants with the lowest energy are assigned a score of 100, highest −0.

Fig. A3 is a series of figures to illustrate combination of immune escape and fitness prior (e.g. infectivity) for continuous monitoring. (A) Snapshot of lineages in terms of fitness prior and immune escape score on respectively from left to right January 17th, 2021, Sep. 1, 2021 and Nov. 23, 2021. Marker size indicates the number of submissions of each lineage. (B) Given a large number of lineages, densities are used instead of points clouds for visualisation. Densities of non-designated and designated variants on Jan. 17, 2021, September 1st 2021 and November 23rd 2021 are represented. The density contour plot is computed by grouping points specified by their coordinates into bins and calculating contours using counts. (C) Progression of the fitness prior (e.g. infectivity) and immune escape scores of main lineages designated by WHO through time from the early snapshot (January 2021) to the later snapshot (September 2021). Each dot represents the position of the center of mass of a given lineage on each month. The left and center plot demonstrates the progression using fitness prior score with and without growth respectively. The right plot shows the progression using only growth.

Fig. A4 is a series of charts showing how EWS flags High Risk Variants ahead of their WHO designation. (A) Cumulative sum of all cases of a given variant lineage (in log scale) over time. Vertical lines indicate the date of WHO designation of a given variant (green dot-dashed) vs. date of flagging by the EWS (red dashed, using a weekly watch-list size of 20 variants). (B). Lead time of EWS detection ahead of WHO designation vs. minimum weekly watch-list size required (in log scale). (C). Detection results (measured in days of lead time vs. WHO designation) from selecting 20 variants per week at random (repeated 100 times) compared with selecting top 20 variants by growth score (light-green cross) and immune escape score (green circle). Boxplots borders indicate 25th and 75th percentiles, horizontal lines indicate median, and whiskers indicate minimal and maximal values. If a variant cannot be detected with growth or immune escape score, the marker is not displayed. (D) Variants detected when using Epitope Score, Semantic Score and Immune Escape Score components of the EWS. The left bar chart displays the number of variants detected by EWS using different scores. The right part visualizes whether a WHO designated variant is detected in advance using different scores, where green dots indicate early detections and grey dots mean the variants are not detected in advance.

Figure S1 is a series of schematic diagrams illustrating machine learning modelling. (A) A transformer language model is pre-trained on all the protein sequences registered in the UniRef100 dataset. Every week, the model is fine-tuned over all the spike protein sequences registered at least twice, so far by the GISAID initiative. Both the pre-training and fine-tuning use the same protocol. Amino acids of a protein sequence are randomly masked. The model predicts probabilities over amino-acids at each residue position, both for residues that were masked and not masked. A loss function evaluates the sum over the masked residues of the log-probability of the correct predictions. A gradient of this loss is computed and used to update the model's parameters so as to increase the loss function. (B) Once fine-tuned, the model is used to compute the semantic change and the log-likelihood to characterise a Spike protein sequence. The output of the last transformer layer is averaged over the residues to obtain an embedding z of the protein sequence. The embedding of the Wuhan strain z_{wuhan} and the embedding of the D614G variant z_{D614G} are computed once for all. The semantic change is computed as the sum of the L1 distance between the z and z_{wuhan} the L1 distance between z and z_{D614G}. The log-likelihood is computed from the probabilities over the residues returned by the model. It is calculated as the sum of the log-probabilities over all the positions of the Spike protein amino acids.

Figure S2 is a series of plots showing cross-neutralization of BNT162b2-immune sera against VSV-SARS-CoV-2-S pseudoviruses bearing the spike protein of selected SARS-CoV-2 variants. Serum samples were obtained from participants in the BNT162b2 vaccine phase-I/II trial on day 28 or day 43 (7 or 21 days after Dose 2). A recombinant vesicular stomatitis virus (VSV)-based SARS-CoV-2 pseudovirus neutralisation assay was used to measure neutralisation. The pseudoviruses tested incorporated the ancestral SARS-CoV-2 Wuhan Hu-1 Spike or Spikes with substitutions present in B.1.1.7+E484K (Alpha), B.1.351 (Beta), P.1 (Gamma), B.1.617.2 (Delta), AY.1 (Delta), B.1.427/B.1.429 (Epsilon), B.1.526 (Iota), B.1.617 (Kappa), C.37 (Lambda), C.37* (Lambda), A.VOI.V2, B.1.517, B.1.258, B.1.160, and B.1.1.529 (Omicron) (Table S.3). (A) Pseudovirus 50% neutralisation titers (pVNT₅₀) are shown. Dots represent results from individual serum samples. Lines connect paired neutralisation analyses performed within one experiment. In total 8 experiments were performed covering the listed SARS-CoV-2 variants always referencing variant-specific neutralisation to the Wuhan reference. (B) pVNT₅₀ against B.1.1.529 (Omicron) are shown. Dots represent results from individual serum samples. Lines connect paired neutralisation analyses performed within one experiment. (C) Ratio of pVNT₅₀ between SARS-CoV-2 variant and Wuhan reference strain spike-pseudotyped VSV. Dots represent results from individual serum samples. Horizontal bars represent geometric mean ratios, error bars represent 95% confidence intervals.

Figure S3 is a series of charts showing results of molecular simulations of RBD binding. The efficiency of spike protein RBD binding to the ACE2 receptor is dictated by the combination of binding energy (A; the lower the better) and size of the interface (B). Both boxen plots depict distribution of these values across performed RBD binding simulations for circulating spike protein variants. Note, that while larger interfaces may be difficult to form, they are also more difficult to break. Strikingly, Omicron, despite its heavily mutated RBD has a relatively large interface and a binding affinity around the 25th percentile of the background distribution (‘Other’).

Figure S4 is a series of plots showing how log-likelihood score corrects for large mutation count. (Left) Snapshot of lineages in terms of log-likelihood ranked without correction for large mutation count and immune escape score on November 23rd 2021. Marker size indicates the number of submissions of each lineage. (Right) Same plot where the log-likelihood ranked without correction has been replaced by its corrected version. Note, that both plots are nearly identical, as highly mutated sequences comprise less than 10% of the entire data set. We observe nearly no change between the plots, with concerning lineages residing on the second Pareto front, except for the emergence of B.1.1.529 (Omicron) as a clear outlier, practically alone in the first Pareto front, due to its high immune escape and extraordinary log-likelihood, given its high number of mutations. Additionally, the conditional log-likelihood score is nearly collinear with expected prevalence of sequence in population (see Fig. S8)

Figure S5 is a series of plots showing combination of immune escape and fitness prior (e.g. infectivity) for continuous monitoring. (Left) Density contour plot of sequences on Jan. 17, 2021. Sequences are split into two groups: WHO designated ones and other non-designated ones. (Center) Density contour plot on September 1st, 2021. (Right) Density contour plot on Nov. 23, 2021.

Figure S6 is a plot showing the maximum lead time of EWS detection ahead of WHO designation vs. required weekly watch-list size. With a weekly watch list of 200 sequences, all WHO designated variants are detected, including Delta.

Figure S7 is a plot showing metrics of anticipated reduction of the immune response. Semantic change and Epitope score accurately segment the variant landscape, allowing to discriminate between variants that do not have immune escape propensity (B.1.429, WT), highly mutated, but neutralizable variants (P.1, B.1.160), and those with high potential for evading immune response (B.1.1.7, AY.1, B.1.351).

Figure S8 is a plot for validating the conditional log-likelihood score. Sequences are grouped into bins based on their submission count and the conditional log-likelihood scores and number of submissions were averaged per bin. The first ten bins correspond to count 1 to 10. The next 10 bins are equally split between counts 11 and 1000 such that each bin has a similar number of sequences. The last two bin contains all sequences having a submission count from 1000 to 10,000 and sequences having more than 10,000 submissions. This shows that the mean conditional log-likelihood of sequences that are observed frequently in circulation is much higher than that of outlier, infrequent sequences.

### DETAILED DESCRIPTION

The variants identified and/or selected by the methods described herein are variants of an originally identified disease associated immunogen. Such an originally identified variant may also be referred to as a native or wild-type and is used as the reference immunogen in the context of this disclosure. For example, in the context of SARS-CoV-2 the originally identified/native/wild-type variant is the variant characterised in Wuhan, China in early 2020 (Lu et al. “Genomic characterisation and epidemiology of 2019 novel coronavirus: implications for virus origins and receptor binding.” 2020; Wu al. “Complete genome characterisation of a novel coronavirus associated with severe human respiratory disease in Wuhan, China” 2020.)

An immunogen refers to a molecule or biological entity capable of eliciting an immune response by an organism's immune system. In an embodiment, the immunogen is a disease associated antigen which gets presented on the organism's antigen presenting cells in association with MHC molecules. In an embodiment, the immunogen is a pathogen or immunogenic material from a pathogen. A variant immunogen herein may also refer to an entire disease causing pathogen, such as a variant strain or SARS-CoV-2, or an immunogenic sequence from such pathogen such as an antigen or epitope that is capable of being presented on the organism's antigen presenting cells in association with MHC molecules. In one example, the immunogen is an immunogenic protein from the pathogen, such as the coronavirus spike protein.

The immunogens identified by the methods herein as existing variants of concern or as yet unidentified potential variants of concerns have several applications in disease surveillance, diagnosis, prevention and therapy. If the immunogens are from pathogens such as influenza, Ebola or coronaviruses then identification and prediction of variants of concerns can have huge utility in surveillance of variants. Hence, the invention also relates to methods of surveillance utilising variants identified or characterised as variants of concern.

Immunogenic compositions, such as vaccines, comprising such immunogens, methods of making such compositions and methods of prevention or treatment using such compositions all form further aspects of the present invention. Each immunogen in the composition may be a peptide sequence. The immunogen(s) in such an immunogenic composition may be nucleic acid sequences such as mRNA sequences. The nucleic acids can be delivered complexed to cationic compounds, such as cationic lipids. The composition may include one, two or several (for example 3 to 10) immunogens selected or identified according to the methods disclosed herein. The immunogen(s) in the immunogenic compositions can also be included in viral vector-based vaccine platforms, such as vaccinia, fowlpox, self-replicating alphavirus, marabavirus, adenovirus (See, e.g., Tatsis et al, “Adenoviruses”, 2004). The effective amount and method of administration of a particular composition can vary based on the individual patient and other factors evident to one skilled in the art.

In some embodiments, the immunogenic composition can be formulated or administered with one or more pharmaceutically acceptable excipients such as stabilising agents, encapsulating agents and buffers.

In some embodiments, the immunogenic composition may contain an amount of an adjuvant such as alum and MPL. In some embodiments, the immunogen is formulated with liposomes or microparticles. A variety of methods are available for preparing liposomes, as described in, e.g., Szoka et al, “Comparative properties and methods of preparation of lipid vesicles (liposomes)”, 1980.

The immunogens identified or selected by any elements within any aspect of the invention can also be used in developing diagnostics and therapeutics. Therefore, these methods can be methods for selection or identification of immunogens for the purpose of developing therapeutics, such as antibodies, or for the purpose of preparing diagnostic assays instead of for the purpose of preparing an immunogenic composition. Such further applications also form aspects of the invention and the embodiments of any aspect discussed above can equally be embodiments of all further aspects discussed herein.

### Machine Learning Model

A method will now be described in which fine-tuned language models perform inference on immunogen variants. Language Models (LMs) are neural networks that were designed for the purpose of predicting words within sentences, accounting for both the individual probability of a word occurring as well as the broader context of the sentence. The approach described herein relies on parallels that exist between natural languages and the syntax of protein sequences. By considering the protein sequence of a given immunogen as a sentence, and the constituent amino acids as the words within the sentence, a properly-trained LM may be used to predict the likelihood of finding different amino acids at a position within a protein sequence given the rest of the sequence. In order for this to be feasible, the LM must be trained on datasets of protein sequences.

By training a LM on large datasets of protein sequences found in nature, the LM is able to predict protein sequences likely to naturally exist. Large deep neural networks such as Recurrent networks or Transformers have been trained on such large general datasets of protein sequences, and the weights of the trained models have been open-sourced. One example of such an open-source trained model would be the Bi-LSTM model with a pre-trained checkpoint released by Hie et. al (Learning the language of viral evolution and escape, 2021), trained on protein sequences sourced from the Virus Pathogen Resource (ViPR) database, National Centre for Biotechnology Information (NCBI) Virus database, and GSAID database. Other machine learning models available include Evolutionary Scale Models (ESM), such as those released by Facebook (registered Trademark) and trained on datasets from UniRef (Suzek et al. “UniRef: comprehensive and non-redundant UniProt reference clusters” 2007). All models and training datasets used in the embodiments are open-sourced, with resources from the NCBI and ViPR databases being available to the public. GISAID is accessible subject to terms of use and a data access agreement. In a preferred embodiment, multiple machine learning models of different types (ESM, BiLSTM, or others) trained on a variety of different datasets, are used. This exhaustive approach is used to minimize the risk of bias across homogeneous models and training datasets. Given a composition of the training set, one includes certain biases in the resultant models. These biases could be avoided, if the underlying distribution of samples (such as protein sequences) were sampled uniformly and exhaustively. However, collection of biological samples rarely follows these rules. Moreover, it is not immediately obvious, which information is conducive for constructing the model of greatest utility. Training multiple models, potentially with varying architectures, allows to overcome the inherent model- and data-bias.

Due to the diversity of protein sequences found in nature, these existing models are not sufficiently granular as to be useful when performing inference on variants of a particular immunogen, such as a virus. Therefore, for this method, existing models are fine-tuned using datasets of known immunogens. By way of example and not limitation, this specification describes an implementation specific to coronaviruses; the skilled person will appreciate that the methods may be adapted for use on any virus or analogous pathogen, or other source of immunogenic material.

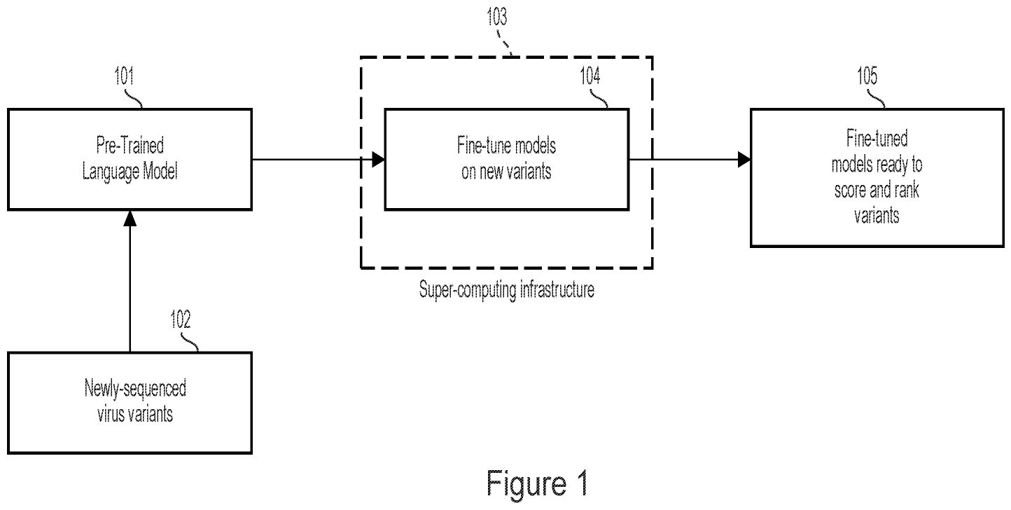

FIG. 1 is a flowchart showing steps in a process for training deep language models. The process is repeated for each of an ensemble of LM. Beginning with a deep language model 101, such as ESM 670M, that has been trained on large datasets of naturally-occurring protein sequences spanning a wide-range of sources not limited by biological grouping, physiological function, or chemical structure, data 102 consisting of newly-sequenced variants of a virus, representing the most up-to-date data available, is used as a training dataset. Example datasets of newly-sequenced variants may be found in the NCBI Virus resource, which aggregate viral data including genomes, nucleotide and protein sequence data. A subset of data from GISAID gathered from published papers is also provided, identical in type to that used. It should be noted that per the GISAID Data Access Agreement, this data is not comprehensive nor sufficient to replicate the training conducted; however, the training data is available in its entirety through GISAID subject to acceptance of the Data Access Agreement and Terms of Use.

The language model is then trained on a super computing infrastructure 103—this infrastructure may take the form of a cluster of graphics processing cards (GPUs), such as NVIDIA A100 GPUs. By training LM 101 on specific dataset 102, the fine-tuning process 103 allows LM 101 to learn sufficient information about the sequences of particular viruses as to be usable for scoring and ranking variants of that particular virus. Depending on the type of model implemented, different libraries may be used for the fine-tuning process 104. For example, for the fine-tuning of Recurrent models, Tensorflow may be relied upon, and Pytorch may be used for Transformer variants.

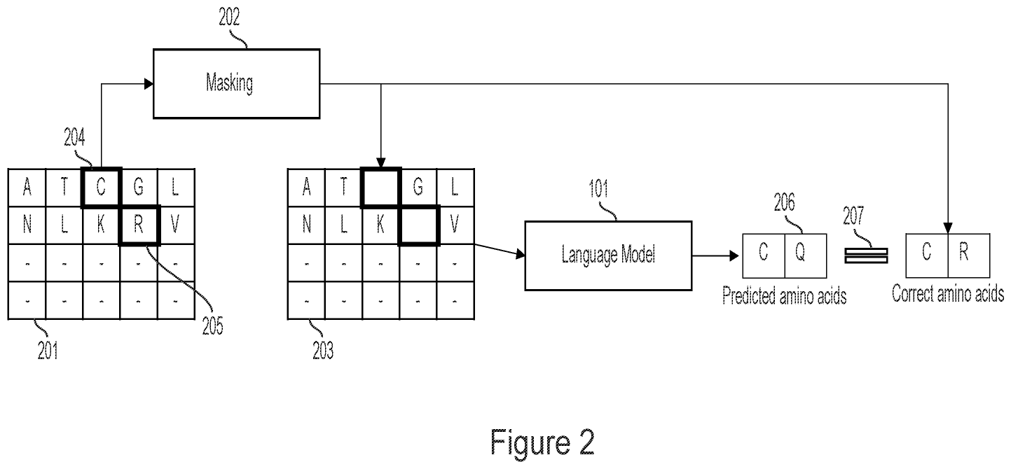

FIG. 2 is a flowchart showing steps of the fine-tuning process 104 of training the language models. The fine-tuning process is an example of self-supervised training of the language models. A batch of protein sequence data—the translations of the variants' DNA (or RNA) sequence—is sampled uniformly and randomly from the newly-sequenced variants dataset 102 at each iteration. If some sequences in the newly-sequenced variants dataset 102 have different lengths, the shorter sequences are padded so that each sequence in the batch gets the same length as the sequence with the maximum length. The input to the LM models is adapted to take into account positions that have been padded. For example, for Transformer models, the input is adapted to avoid computing attention and for Recurrent networks the model is not rolled over padded elements. Care is also taken not to consider padded elements in the loss function when updating the weights of the models.

FIG. 2 illustrates the process for a single sequence 201 of the batch. After sampling, a masking process 202 is applied, selecting data identifying one or more amino acids within the sequence at random, and masking the data point(s). This results in masked protein sequence 203. As shown, the data identifying amino acids at positions 204 and 205 have been masked, essentially ‘de-identifying’ them and hiding them from LM 101. This provides the LM with training data to fine-tune the LM. The masked protein sequence data 203 provides an input and the original protein sequence data provides a desired output.

The masking for training the language models varies between the Recurrent models and the Transformer models, such as ESM 1-b.

For the Recurrent models one position in the sequence is masked and the sequence on either side of the masked position is input into the Recurrent model. The amino acids are selected and masked at random from the sequence and, for each masked sequence, the logarithm of the probability is returned by the model. The calculated probability relates to the amino acid that was initially at the position 204 or 205 being masked. The loss function for this sequence is then calculated as the negative sum of all each of these logarithms of probability (cross-entropy loss), and the gradient of this loss function is then calculated with respect to the network weights.

The person skilled in the art will appreciate that language models as used may be trained according to a variety of training strategies, and that the proper training strategy may be selected according to the data. By way of example and not limitation, language models as used herein may be trained as described. For Transformer-based models, such as ESM 1-b, multiple positions in the protein sequence are masked. The loss value is taken to be the whole-batch average cross-entropy loss between the model's predictions/tokens, (x1, . . . , xn) and an output sequence of log probabilities (y1, . . . , yn) which are optimized using a probabilistically masked language modelling. A percentage, say 15%, of locations on the sequence are chosen for masking. For each position chosen for masking, there are three possibilities: 1) the amino acid is replaced by a mask token (80% chance) 2) the amino acid is replaced by a random amino acid (10% chance) 3) the amino acid is unchanged (10% chance). This approach to masking is explained in ‘Biological structure and function emerge from scaling unsupervised learning to 250 million protein sequences' (Rives et al. “Biological Structure and Function Emerge from Scaling Unsupervised Learning to 250 Million Protein Sequences.” 2020).

A variety of techniques may then be implemented to update the weights of the LM 101 based on the loss function. In one embodiment, classical gradient descent may be used, in which this gradient is multiplied by a fixed coefficient, called the learning rate. This multiplied gradient is then subtracted from the network weights, updating them. In other embodiments, techniques such as ADAM may be used, in which running average statistics are maintained over past gradients in order to compute an adaptive learning rate.

After the LM has been trained, a masked sequence 203 may be presented to LM 101 as input data and the LM will predict the probability of different resulting protein sequences. Predicting the probability of a protein sequence is performed differently between Recurrent networks LMs and Transformers models.

For the Recurrent models, in order to predict a likelihood for a particular protein sequence as a whole, inference is performed with a single mask applied sequentially along the protein sequence. In other words, for each masked position, the data either side of the masked position is entered into the LM. For each masked protein sequence, the LM 101 views the entire protein sequence, and derives the log-likelihood of the actual amino acid being in the masked position. As noted above, this is performed for every amino acid data point in the overall protein sequence. The resulting log-likelihoods of the predicted probabilities for each position are then summed, giving the overall log-likelihood of a given protein sequence—this overall log-likelihood can be thought of as a measure of how likely the sequence is to occur in nature.

For Transformer based models, no masking is applied, and the model returns a sequence of log probabilities for each position in the input sequence. Again, the resulting log-likelihoods of the predicted probabilities for each position are then summed, giving the overall log-likelihood of a given protein sequence In some embodiments, to remove the distortion of the summed log-likelihood values caused by variants of different lengths, the overall log-likelihood may be calculated as an average of the log-likelihoods for each amino acid across the sequence.

The process shown in FIG. 2 is performed for each of a plurality of models to form an ensemble of trained LM models.

The log-likelihood of a particular protein sequence will likely be different for each model. For each model, the log-likelihood values are first normalized based on the training data. The values output by the ensemble of models are then averaged, forming a final log-likelihood of the protein sequence in question.

### Variant Scoring & Ranking

The ensemble of language models fine-tuned as described above may now be used to generate rankings of variants in terms of the danger they may pose. This ranking is generated through a scoring of the variants according to several criteria.

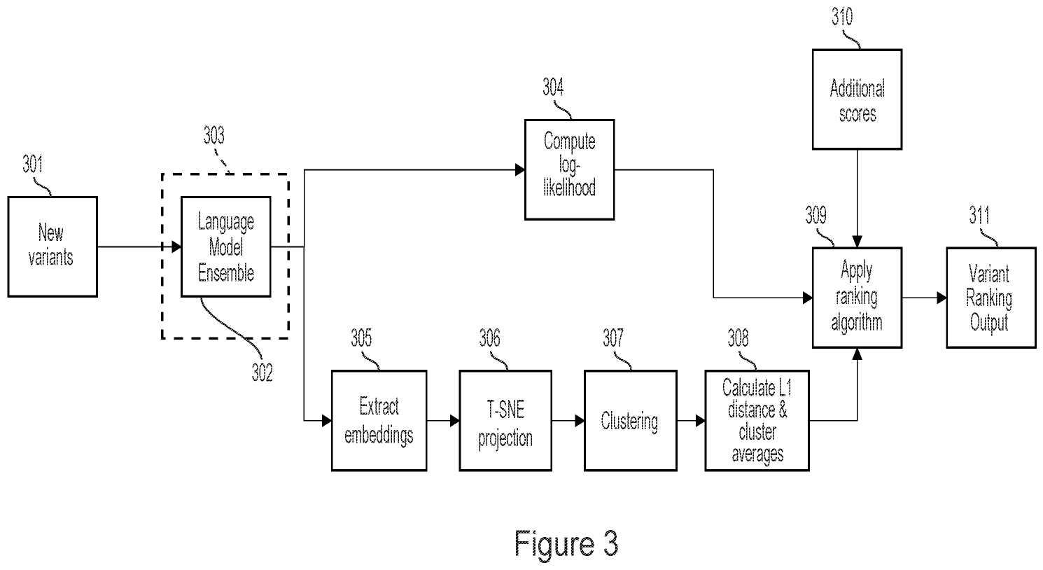

FIG. 3 illustrates the process by which new variants are scored. After sequencing, the new variants have their DNA/RNA sequences translated into protein sequences. Data representing these protein sequences are input 301 to an ensemble of language models. This ensemble 302 comprises multiple language models such as LM 101, each having a different architecture and having been fine-tuned separately as described above. The ensemble 302 may be stored and processed on a super-computing infrastructure 303—in one implementation, this infrastructure may comprise a high number of GPUs and CPU cores. The input 301 is fed into each language model 101 within the ensemble of language models 302. The language models each calculate an overall log-likelihood for the input protein sequence using the methods described above. As explained above, the method differs slightly between Recurrent LMs and Transformer-based models. Each language model is different from each other and this makes it highly unlikely that, should any errors or blind spots develop in one language model, the other language models will share the same issues. In this way, the ensemble approach ensures consistently reliable analysis.

In step 304, the overall log-likelihoods thus generated by each model are averaged—this provides an overall log-likelihood for each variant. The overall log-likelihood of each variant determines the likelihood that this sequence may exist in nature. This value can be understood as a measure of self-consistency of the protein sequence and its likelihood to occur in nature and may therefore form a first scoring criterion (Score 1) from which the overall ranking of the variant may be calculated.

Each model 101 provides a second output, which does not consist of the probabilities relating to each variant, but rather is a knowledge representation processed up to a given layer by the model 101. In step 305, this knowledge representation is extracted from an output feature map at an intermediate (hidden) layer of the LM. This knowledge representation will be referred to as an ‘embedding’.

For Transformer models, the ‘embedding’ is an output feature map of the penultimate (last transformer layer) of the LM.

As explained above, for the Recurrent models, a separate inference is performed for each amino acid location in the protein sequence of the variant. Accordingly, a separate embedding exists as the output feature map of a penultimate layer for each inference performed. By averaging the embeddings (output feature maps) across all amino acid positions within the sequence, a sequence embedding may be created.

In the embodiment described above, the LM layer in question is the final embedding layer of a Recurrent model or the last transformer layer of a Transformer-based model, however in other embodiments other intermediate layers may be selected. In one embodiment, this selection may be based on criteria such as whether the resulting embedding would better show separation between existing variants and randomly mutated variants. The person skilled in the art will recognize that a variety of subjective or quantitative characteristics may be used as selection criteria, and that the selection of the appropriate layer could be considered a hyper-parameter—i.e. a parameter that controls the process.

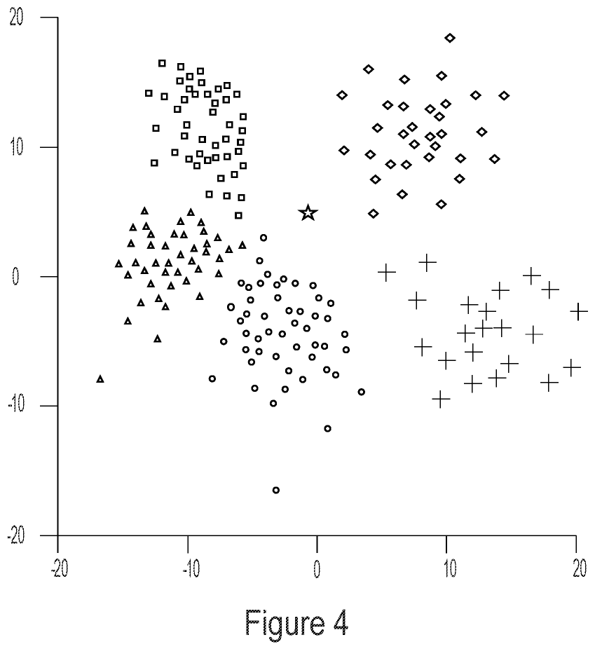

An overall embedding vector for the protein sequence of a variant is determined for each LM. These embedding vectors express categorical variables—variables that can take on one of a limited number of values, in this case protein sequences representing variants—as continuous vectors, each representing a respective variant as a point in N-dimensional space. Points representing variants with a higher degree of similarity may be grouped together within this space. This allows not just the probabilities of the individual variants, but also their location in relation to, and similarity with, other variants, to be leveraged as a source of insight, as detailed below in connection with FIGS. 3 and 4.

At this point in the process, for a particular variant, each model 101 has generated a separate overall embedding vector. To perform scoring and ranking operations, the embedding vectors from the different LMs must be turned into a single set of data. The embedding vectors of all models in the ensemble of models 302 are concatenated to form a concatenated embedding vector. The resulting embedding vector for each variant thus corresponds to the concatenation of the respective overall embedding vectors from each model 101 of the ensemble of models 302.

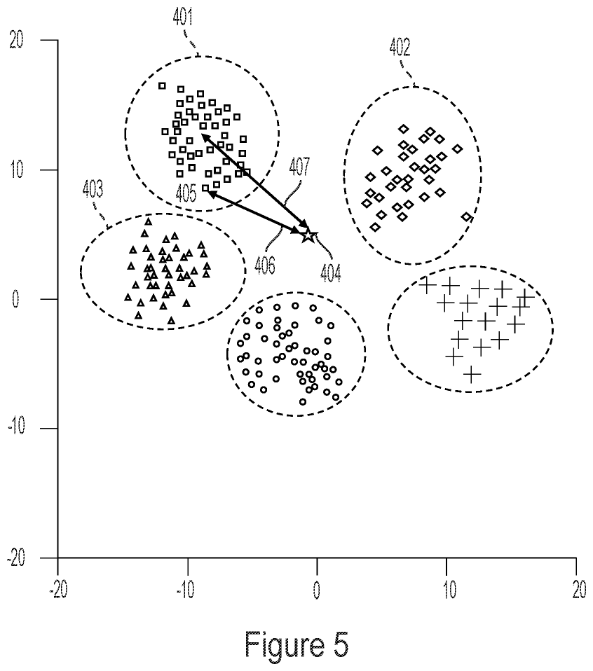

The embedding vectors generated as described are multi-dimensional data—for example, embedding vectors retrieved from an output feature map of a Transformer model may have 1024 dimensional values—so in order to enable the recognition of similarities between data points, and to facilitate both visualization and efficient calculation, this data is represented in lower dimensions. To this end, in step 306, a dimensionality reduction is performed. In one implementation, this may involve performing a T-SNE (t-distributed stochastic neighbourhood embedding) computation on the embedding vectors generated previously, in order to project a two-dimensional visualization of the data points representing each variant. The skilled person will appreciate that other dimensionality reduction techniques are available and may be applied, and that the data may be projected into other lower dimensional spaces such as three-dimensional space. The result of this reduction to two dimensions is shown in FIG. 4, which illustrates an exemplary T-SNE projection. The variants tend to cluster in T-SNE space, and the lines in FIG. 5 show the clusters. This T-SNE projection should display not only the embeddings of the new variants 401, 402, 403, but also the embeddings of a known variant 404.

Once the T-SNE projection has been computed, in step 307 clustering may be performed in the projected two-dimensional space. T-SNE plots will naturally tend to display clusters of data points, as variables that have been grouped together in at least one dimension at the embedding stage will likely be represented in close proximity in the T-SNE space. To identify groups of data points representing variants, in one embodiment k-means clustering may be used. This method of clustering partitions the entire set of data points into a fixed, defined number (k) of clusters. The clustering algorithm identifies k ‘centroids’, which serve as the location (real or imaginary) representing the centre of each cluster. Each data point is then allocated to the cluster with the nearest mean, i.e. where the lowest squared Euclidean distance to the centroid is lowest. An example of a T-SNE plot in accordance with this embodiment may be seen in FIG. 5. The embedding vectors for variants displaying similarities in their protein sequences are grouped into clusters 401, 402, and 403. The embedding for the known variant is marked as 404.

In step 308, several calculations are performed to calculate scores for the variants.

Firstly, the L1 distance 406 between the overall embedding vector representing anew variant 405 and the overall embedding vector representing the known variant 404 may be calculated. The L1 distance 406 is the sum of the magnitude of the vectors—it is calculated as the sum of the absolute differences of the components of the vectors.

The L1 distance 406 between the overall embedding vector of each of the new variant 405 and the overall embedding vector of the known variant 404 correlates to the immune escape potential of the new variant in question. The L1 distance 406 corresponds to how far apart the data points in question are—this in turn correlates to the dissimilarity of the two variants. A variant that is less similar to the known variant is less likely to be captured by an immune response provoked by—and targeted at—the known variant. Consequently, the L1 distance 406 provides a metric as to how likely a new variant is to evade an immune response. This L1 distance 406 forms a second criterion (Score 2) from which the overall ranking of the variant may be calculated. As the L1 distance is the sum of the absolute differences of the components of the vectors, this calculation is performed in the embedding space.

The T-SNE projection allows data points representing similar variants to be clustered together, providing a visual indication of how similar new variants are, not only to the known variant (which is calculated using the L1 distance as described) but to each other. This clustering of variants on the T-SNE projection also allows for calculations to be performed based not only on a given new variant, but also based on the other new variants within the same cluster.

The log-likelihood of all the variants within the same cluster may be averaged. This average log-likelihood provides the third criterion (Score 3) from which the ranking of the variant may be calculated.

Similarly, the L1 distance between the known variant and the new variants within the same cluster may be averaged, to give an average L1 distance 407 for the cluster, forming the fourth criterion (Score 4) from which the ranking of the variant may be calculated.

Criteria 3 and 4 provide additional information beyond that provided by examining only a specific new variant and not the variants within the same cluster. A variant is likely to be concerning when not only it, but also variants around it, have high scores. A variant that has a high log-likelihood, but that is surrounded by variants with low log-likelihoods, may on its own be likely to exist but is unlikely to mutate into a second similar variant, and therefore poses a lower overall risk.

In one implementation, the criteria from which an overall ranking of a variant may be calculated is therefore:

Score 1: log-likelihood of new variant.

Score 2: L1 distance between known variant overall embedding vector and new variant overall embedding vector.

Score 3: average log-likelihood of new variants in the same cluster.

Score 4: average L1 distance between the overall embedding vector of the known variant and the overall embedding vectors of the new variants in the same cluster.

Additional human expert scores 309 may also be provided but are not required. Additional scores are described below in further embodiments. If provided, these may be considered when overall ranking is performed.

In step 310, a ranking algorithm is applied to the scores generated as above. In one implementation, this ranking algorithm may apply a “league score” format to the scores generated. In this implementation, the variant with the highest value for Score 1 is given a maximum value of points, the variant with the second highest value is given a score one point lower, the variant with the third highest a score one point lower than that, and so on. This format may be applied to all of the scores generated, and the points awarded to each variant may be summed to provide an overall score for that variant. In step 311, these overall scores should provide a basis for ranking the new variants from most to least concerning.

### Further Embodiment—Further Score Based on Epitope

In a further embodiment of the invention, an epitope score, illustrated at step 310, may be calculated during the scoring process described in connection with FIG. 3. This further score may provide information relating to epitopes or antigenic determinants within a variant which are recognized by the host immune system, such as by antibodies created by the immune system of a host.

In order to generate this further score, epitopes are identified from information present and available in the art at the time. For example, in the case of coronavirus, the epitopes may be identified from published research on coronavirus epitopes. Based on this information or research, epitope(s) of the variant in question may be identified, for example, on the spike protein of the variant.

A weighting is then allocated to sections of the amino acid sequence of the variant based on the nature of its epitope(s). Different weightings may be allocated depending upon whether the sequence section is: i) a known epitope, ii) hypothesized but not known with certainty to be an epitope, or iii) a portion of the amino acid sequence relevant to the spatial structure of an epitope. Sequences relevant to spatial structure can be (for linear epitopes) sequentially neighbouring amino acid residues, or sequential segments that are brought together in spatial proximity when the corresponding antigen is folded (for conformational epitopes).

Following this, mutations in a naturally-occurring or de-novo variant of the chosen immunogen are identified compared to the chosen native/wild-type/initially identified variant, with their location being noted. The further score for the naturally-occurring or de-novo variant, is then calculated, based on the location of each mutation. The score is computed in accordance with the weighting of the different sections of the sequence described above. In one embodiment, the surface availability of the position in question may also be used to calculate this score.

In the case of the SARS-CoV-2 spike protein, previously resolved structures of neutralizing antibodies (nAbs) may be mapped onto the S protein based on publicly available resolved 3D structures of the nAbs. Previously resolved nAbs are described in Barnes et al. “SARS-CoV-2 neutralizing antibody structures inform therapeutic strategies” 2020, Dejnirattisai et al “The antigenic anatomy of SARS-CoV-2 receptor binding domain.” 2021, Ju et al. “Human neutralizing antibodies elicited by SARS-CoV-2 infection”, 2020, Yan et al. “Structural basis for bivalent binding and inhibition of SARS-CoV-2 infection by human potent neutralizing antibodies.” 2021. The number of known nAbs whose binding epitope is affected by a distinct SARS-CoV-2 variants' mutations may be defined as the epitope score.

Calculating the epitope score may include enumerating positions on the spike protein sequence or another relevant reference immunogen sequence that are expected to be involved in binding of the immunogen by antibodies, specifically neutralizing antibodies. An epitope in the immunogen sequence that can be bound by multiple antibodies is only counted as one epitope. The epitope score may be generated by counting the number of epitopes that contain mutations. In this case, an epitope with multiple mutations is only counted once for the epitope score. For example, if there are 7 mutations that affect epitopes, but two of the mutations are on the same epitope, the epitope score may be assigned as 6. This score accounts for the differences in danger posed by variants of a chosen immunogen based on the degree to which the mutation affects the epitope(s). Mutations affecting the epitope of a variant are likely to increase the danger posed by the variant substantially due to the potential for immune escape. A variant having an epitope that differs, due to mutation, from the epitope of a known variant will more likely not be recognized to the same degree by the immune system. The mutated variant may therefore not be affected to the same degree by antibodies released as part of the immune response.

### Further Embodiment—ACE2 Binding Score Based on ACE2 Binding with Receptor Binding Domain

Transmissibility and immune escape potential of a variant is affected by the binding affinity of the variant with its human receptor, angiotensin-converting enzyme 2 (ACE2), which is necessary for host cell infection. Accordingly, to represent this factor, an ACE2 binding score may be generated by in silico modelling. In the example of SARS-CoV-2 spike protein, the interaction between a variant S protein (the main antigen component) and the ACE2 protein may be estimated through repeated docking experiments as explained in more detail below. In some embodiments, in order to reduce the required computational resources, the spike protein structure modelling may be restricted to its RBD domain i.e. the domain known to be directly binding to the ACE2 receptor.

In one example, a number of receptor-binding domain (RBD) differentiated variants (proteins with differences between each other in the RBD domain), including the wild type, were selected for in-silico simulation. For each variant, a folded protein structure is simulated based on an existing model of a related variant, such as a wild-type variant. From this existing model, further structures, in some examples at least 500 structures, for the protein are generated using a conformational sampling algorithm with a view to identifying a protein structure for the variant with a lowest energy. These structures are further optimized with a probabilistic optimization algorithm, a variant of simulated annealing, aiming to overcome local energy barriers and follow a kinetically accessible path toward an attainable deep energy minimum with respect to a knowledge-based, protein-oriented potential. The knowledge-based protein-oriented potential is a function calculated based on known energies of different protein/amino acid configurations that estimates the energy of the protein structure.

For each structure, it is possible to estimate the change of binding energy when the interface forming chains are separated from the human receptor, versus when they are complexed, normalized by the solvent accessible area buried at the interface. A median change in binding energy per RBD variant is determined as a metric. Each metric is normalized by the metric of the wild type, which is considered to have no mutation on its RBD. Accordingly, the ACE2 score for the wild type is set to 1.

In some non-limiting examples, the approach described above may be implemented using available software such as RosettaDock. Computational docking experiments using RosettaDock are described in Marze et al., Efficient flexible backbone protein-protein docking for challenging targets, 2018. A further paper that describes i) generating template-based structural models using related homologs of known 3D structure and an automated search ii) exploring interactions between the structures and iii) modelling of resulting complexes is Quignot et al. InterEvDock3: a combined template-based and free docking server with increased performance through explicit modeling of complex homologs and integration of covariation-based contact maps, 2021.

Compared to conventional energy metrics, the surface area used above is less sensitive to local optimization pitfalls (e.g. side chain packing), and it is more robust across multiple samples, and generally requires less computational resources to compute accurately.

### Further Embodiment—Further Growth Score Based on Growth

A growth score may be calculated from the GISAID metadata. For a given date, growth may take into account a number of sequences relating to a particular variant that have been submitted to the data set within the last eight weeks. For each variant, its proportion among all submissions is calculated for the eight-week window and for the last week, denoted by rwin and rlast correspondingly. The growth of the lineage is defined by their ratio, rlast/rwin, measuring the change of the proportion. Having values larger than one means that the lineage is rising and smaller than one indicates declining.

### Scaling Scores and Merging to Generate Immune Escape Score and Infectivity Score

The L1 distance in embedding space, log-likelihood, epitope score, ACE2 binding score and growth have all different scales and units. Thus, these scores cannot be compared directly. To make comparisons possible, a scaling strategy is introduced. For a given metric m, all the variants considered are ranked according to the particular metric. In the ranking system used, the higher rank the better. Variants with the same value will get the same rank. The ranks are then transformed into values between 0 and 100 through a linear projection to obtain the values for the scaled metric. Each of the scores is scaled according to this strategy.

An immune escape score is computed as the average of the scaled L1 distance and of the scaled epitope score.

An infectivity score is computed as the average of the scaled log-likelihood, the scaled ACE2 binding score and the scaled growth scores.

The log-likelihood may be calculated using a modified scaling method in order to take into account the number of mutations. An increased number of mutations may strongly affect fitness resulting in a lower log-likelihood. However, as there are variants that are observed in the wild that include a large number of mutations, it is hypothesised that some variants with a large number of mutations may in fact be fit. To account for this, the ranking of log-likelihood for the purposes of calculating a log-likelihood score may rank each variant, having N mutations against variants that have a N-m mutations, where m is a predetermine value. In one example, the log-likelihood score is calculated by ranking a variant having N mutations against variants having M=min(max(0, N−10), 50) variations. That is to say that if a variant had 20 mutations, it would only be ranked against variants having at least 10 mutations. In each group, the ranks are transformed into values between 0 and 100 through a linear projection to obtain the values for the scaled log-likelihood score. Experimentation suggests that this method of generating the log-likelihood score is generally robust to varying the value of m.

Further Embodiment without Dimension Reduction

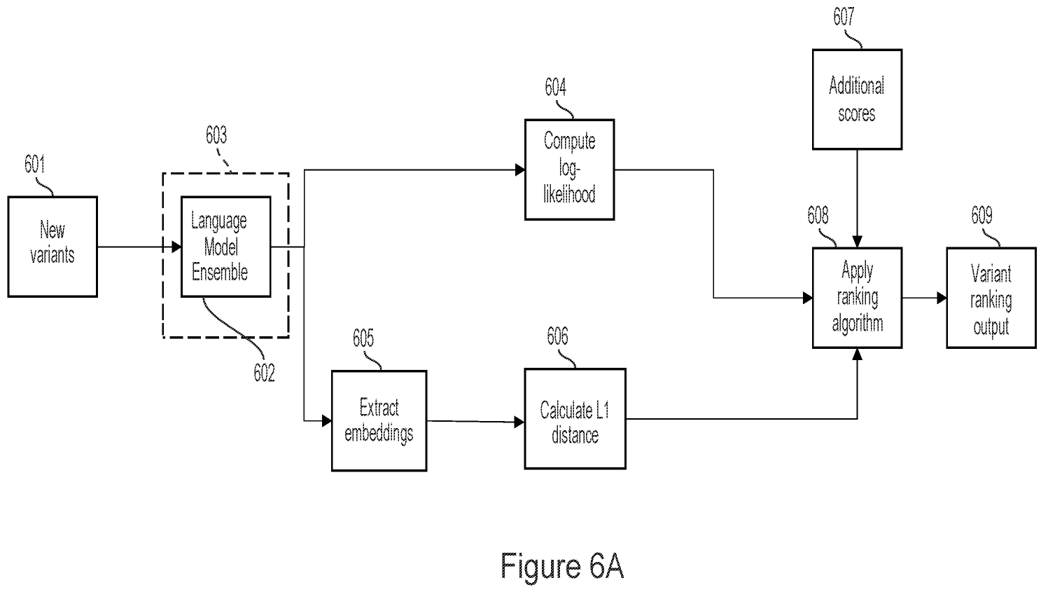

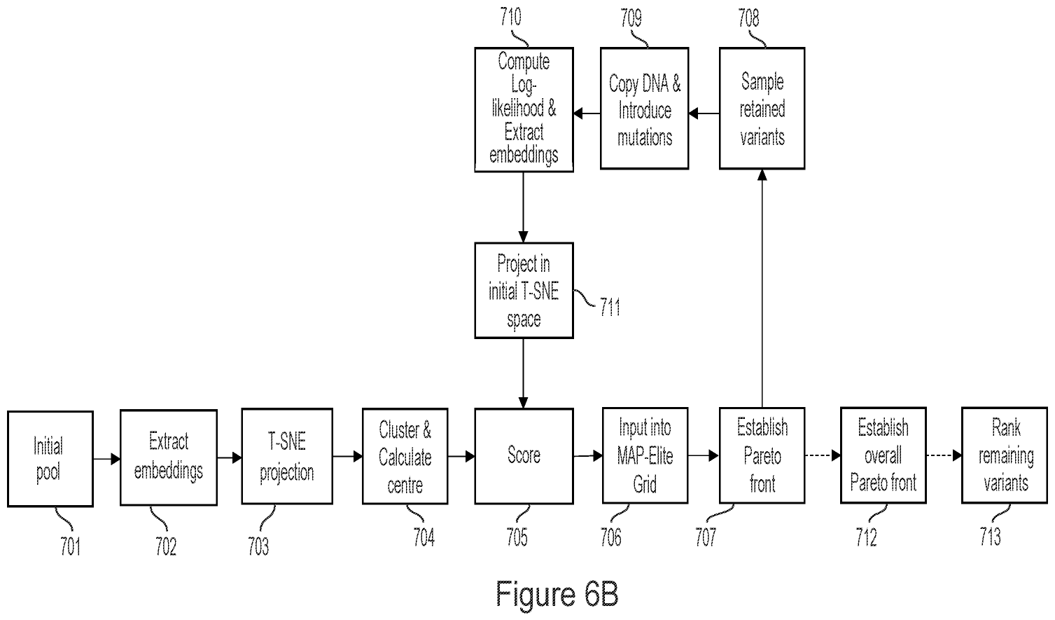

FIG. 6A is a schematic diagram of a ranking process according to a further embodiment. In this further embodiment, the T-SNE projection and clustering in projected two-dimensional space are not performed.

In step 601, similar to step 301, data representing a reference protein sequence and variants associated with the reference protein sequence are input to an ensemble of language models 602 running on a computing architecture 603. In one example, the reference variant may be the original SARS-CoV-2 spike protein characterised in Wuhan and the variants associated with the reference protein sequence may be other SARS-CoV-2 spike protein sequences including mutations that have subsequently been discovered, such as for example, the variants identified by the WHO as variants of concern. This ensemble 602 comprises multiple language models such as LM 101, each having a different architecture and having been fine-tuned separately as described above.

The language models each calculate an overall log-likelihood for the input protein sequence using the methods described above. As explained above, the method differs slightly between Recurrent LMs and Transformer-based models. In step 604, the overall log-likelihoods thus generated by each model are averaged—this provides an overall log-likelihood for each variant. The overall log-likelihood of each variant determines the likelihood that this sequence may exist in nature.

As before, each language model 101 provides a second output, which does not consist of the probabilities relating to each variant, but rather is a knowledge representation processed up to a given layer by the model 101. In step 605, this knowledge representation is extracted from an output feature map at an intermediate (hidden) layer of the LM. This knowledge representation is again referred to as an ‘embedding’. The extraction of embeddings and generation of embedding vectors was described in connection with FIG. 3 and this description is not repeated.

At step 606 a measure of distance is calculated for each variant. The measure of distance for a variant is determined in the embedding space of the embedding vectors and may be determined as a Euclidean distance between the embedding vector of the reference protein sequence and the embedding vector of the variant. In further implementations, the measure of distance may be calculated with reference to additional protein sequences in addition to the reference protein sequence. For example, in the example of SARS-CoV-2, the measure of distance may be determined as a sum of the Euclidean distance between the variant and the original SARS-CoV-2 spike protein characterised in Wuhan and the Euclidean distance between the variant and the D614G variant, which had become the dominant variant in the pandemic around July 2020. Additional (i.e. more than two) variants may be added and different variants may be used as a reference point for the measure of distance depending upon the use case.

As described above, at step 607 additional scores may be generated. These scores include the epitope score, ACE2 binding score, and growth score.

At step 608, the scores are scaled and combined to create an immune escape score and infectivity score for each variant as described in the section above ‘Scaling Scores and Merging to Generate Immune Escape Score and Infectivity Score’.

At step 609, a Pareto score is calculated to generate a variant ranking output. The Pareto score is computed by forming Pareto fronts based on the immune escape score and infectivity score for each variant. A first Pareto front is formed that corresponds to a set of variants for which there does not exist a variant that has both a higher immune escape score and a higher infectivity score. Second and subsequent Pareto fronts are formed by removing the variants that formed the previous Pareto front from the set and forming a new Pareto front. As variants are removed they are assigned a value according to the number of the Pareto front that they were a member of. This process is repeated until all variants have been assigned to a Pareto front. A linear projection is then performed such that variants from the first Pareto front obtain a score of 100 and variants from the last Pareto front are assigned a score of 0.

In some embodiments, a lineage is identified based on the Pango nomenclature system (Rambaut et al., “A dynamic nomenclature proposal for SARS-CoV-2 lineages to assist genomic epidemiology.” 2020) and the Pareto score each sequence belonging to a given lineage is averaged in order to give Pareto scores for specific lineages.

### Vaccine Design

Provided herein is a method for identification of immunogens capable of generating a broad immune response effective against several variants of the target disease or pathogen. The target pathogen may be a virus, such as a coronavirus, such as SARS-CoV-2, with the immunogen generating an immune response effective against several variants of this pathogen. This can, in effect, be seen as an immunogen identification as well as optimisation method.

Also provided herein is an immunogenic composition comprising one or more immunogens identified by the immunogen identification methods of this disclosure. The immunogenic composition may be a vaccine composition; the disclosure here regarding features of one applies equally to the other.

Immunogenic compositions of this disclosure are preferably for use in a method of vaccinating or treating a subject, the vaccination or treatment method comprising selecting one or more immunogens identified as per any of the identification methods of this disclosure.

Finding such an immunogen that would elicit a broad immune response, for example a broadly neutralizing antibody response, requires diverging from naturally observed sequences in order to produce such a protein which “resembles” a wide range of variants of the immunogen. This in and of itself is not an easy task, as the “similarity” metric is not intuitively obvious, and may differ between systems (Crowe, “Principles of Broad and Potent Antiviral Human Antibodies: Insights for Vaccine Design.” 2017; Sevy et al. “Integrating Linear Optimization with Structural Modeling to Increase HIV Neutralization Breadth.” 2018; Schey et al., “Multistate Design of Influenza Antibodies Improves Affinity and Breadth against Seasonal Viruses.” 2019). In most cases, candidate vaccines are produced by a shotgun, trial-and-error approach, in which expert-selected features of the known immune threats are selected and mixed together. The method provided herein seeks to improve both the speed and accuracy of this process, replacing the prevailing approach with one that begins with first principles and proceeds in a logical and sequential way, identifying as vaccine candidates immunogens similar to the maximal number of variants.

The vaccine design platform provided by this method is capable of automatically including and processing all new variants as they are discovered and sequenced, and through automated machine learning methods assessing their variability and potential dangerousness, as measured through their immune escape potential.

The platform can additionally design, in a fully data-driven and automated way, optimized vaccines aiming to address all known virus variants. Vaccines designed according to the following method may also address as yet unobserved but potentially dangerous variants. A variant is characterized by the nucleic acid (DNA or RNA) sequence of the gene encoding the immunogen; the protein (or set of proteins), present in the pathogen (virus), visible to the immune system, such as, but not limited to, influenza's hemagglutinin. The protein sequences of these newly designed immunogens (vaccine candidates) make it possible to promote an effective antibody immune response to protect recipients from all known variants.

The world's ever-growing population, combined with increasing international mobility and threats posed by climate change lead to the emergence and rapid spread of potentially species-wide diseases, causing pandemics as observed with COVID-19. The static vaccine design paradigm, focusing on designing a single vaccine once and for all, is no longer a viable strategy. Viruses evolve to evade immune response, making it infeasible to keep track of all the variants individually; furthermore, it is not possible to reformulate vaccines yearly, for each region separately.

The example provided below regarding SARS-CoV-2 is just a case study, provided by way of example and not limitation, for a broad system proposed in this application. This system, given the base data on pathogen variability, without need for annotation with virulence, infectivity or pathogenicity data, identifies such variants of the immunogens, which are (a) evolutionarily plausible, (b) similar to dangerous variants in terms of anticipated immune response, which can be formulated in the potential forward looking vaccine.

These criteria may be used to design vaccines from first principles, taking into account both existing variants and de-novo ones designed as described previously. To do so, an evolutionary-based multi-criteria optimization algorithm is relied upon. Using this algorithm, immunogen sequences may be designed that closely resemble variants close to the centre of the variant clusters projected in embedding space. The intuitive idea is that vaccines should be as “close” as possible to the centres of as many clusters as possible, so as to be effective against the broadest range of variants, while at the same time be properly-functioning (i.e. evolutionarily ‘fit’) virus vaccines, such that the immune system learns from appropriate and effective coronavirus spike proteins. For the purposes of introducing mutations into the nucleotide sequence of a variant, the algorithm uses the natural mutation probabilities observed in nature for coronaviruses. Furthermore, this method has the capability to evolve not only performing vaccines (i.e. vaccines with high scores on all metrics, including distance to centroids), but also diverse ones (vaccines that encode protein sequences with different properties). Optimizing for diversity in addition to quality prevents the algorithm from converging prematurely on a single set of vaccine candidates; it thus drastically improves the expected metrics of the proposed vaccine candidate sequences.

### Optimization Engine to Design Vaccine Candidates

In a further embodiment, the method includes a process for designing a broad-spectrum vaccine based on the de-novo variants. This embodiment will now be described with respect to FIG. 6B.

The vaccine design process starts at 701 with a pool of known variants. In step 702, the embedding vectors for each of these variants are computed. As described previously, for Recurrent models this step comprises the generation, in each of an ensemble of finely-tuned models 302, of an embedding vector for each amino acid across all positions in the protein sequence of each variant. These embedding vectors are then averaged, generating an averaged embedding vector for the model. For Transformer based models, the embedding vector is the output feature map of the last transformer layer. The embedding vectors for a variant from all models are concatenated to provide a concatenated embedding vector for the variant.

In step 703, a dimensionality reduction is performed on the concatenated embedding vector for each variant, allowing the higher-dimensional concatenated embedding vectors to be displayed in a lower-dimensional format. In one embodiment this dimensionality reduction may take the form of a T-SNE computation, projecting the data in two dimensions. The skilled person will, however, appreciate that other dimensionality reduction techniques are available and may be applied as suitable.

With each variant now represented as a data point in a lower-dimensional projection, in step 704 clustering may be performed, partitioning the overall dataset into meaningful clusters of interest. This has the effect of grouping data points (and therefore variants) with similar characteristics close together. By performing this clustering operation, a centroid may be calculated for each cluster displayed on the projection, this centroid being a mean position of variants in the cluster that was identified in T-SNE space. The centroid is a position in the embedding vector space.

The calculation of the centroid for each cluster allows the design of a broad-spectrum vaccine. The use of embedding vectors and clustering in the data projection means that points close in the embedding space represent virus variants that share similarities. Accordingly, the protein sequences of variants within the same cluster are likely to be similar, and the variants will display similar biological characteristics. This being the case, a vaccine candidate (variant) that has an embedding vector that places it at or close to the centroid of a cluster will likely trigger an immune response that would neutralise the widest possible range of variants in that cluster. Following this logic, the object of the optimization phase of the vaccine design process is to design a vaccine candidate—a de-novo variant—that would have an embedding vector as close as possible to the centre of as many clusters as possible. By doing so, the broadest number of variants in the broadest number of clusters would be neutralized by the immune response triggered by the vaccine candidate thus designed.

In step 705, at least one score is calculated for each variant in the pool. In one embodiment, the first criterion for which a score is calculated is the log-likelihood of the variant. This is known from the final output of the machine learning model, as described above, and provides a measure of the strength and competence of the virus.

Further criteria may rely on the previous calculation of the centre of each cluster. For each variant, L1 distances between the embedding vector of the variant being scored and the calculated centroid of each cluster is computed. As the L1 distance is the sum of the absolute differences of the components of the vectors, this calculation is performed in the embedding space.

Similar to above, the L1 distance provides a measure of similarity: in this particular instance, a lower L1 distance correlates to how close the variant in question is to the centroid of the cluster. As noted, a variant similar to the centroid of a cluster will likely trigger an immune response that neutralizes a broad range of variants in that cluster. Consequently, a variant close to—and therefore similar to—the centroid of multiple clusters will likely trigger an immune response that neutralizes a broad range of variants in those multiple clusters.

The scores of interest for each variant are therefore:
Log-likelihood
L1 distances to each cluster centroid

As the L1 distances in question are between the variant being scored and the centroid of each cluster, the number of scores of interest will therefore depend on the number of clusters that are present in the T-SNE space. In one embodiment, the number of clusters may be 3, resulting in four scores of interest:
1. Log-likelihood of the variant
2. L1 distance between the variant and the centroid of cluster 1
3. L1 distance between the variant and the centroid of cluster 2
4. L1 distance between the variant and the centroid of cluster 3

These scores may then be used to rank the variants, which may be done in a similar manner to that described in the ranking step of the previous embodiment. It must be stressed that the rankings for score 2, relating to the L1 distance to the centre of the cluster, are inverted. Therefore, a variant with a lower L1 distance to the centre of the cluster will have a higher ranking for this score than will a variant with a higher L1 distance to the centre of the cluster.

The variants must now be entered into an optimization algorithm capable of maintaining a broad set of solutions, such that an optimal vaccine across multiple metrics—in this embodiment, log-likelihood and similarity to multiple variants—may be identified. In one embodiment, the algorithm used for this purpose may be an MAP-Elite algorithm.

In one embodiment of the vaccine design process, the method defines the MAP-Elite space by a pair of descriptors, the descriptors corresponding to the two components of the projection of the embedding of a given vaccine candidate in the T-SNE space defined by the pool of initial variants. This therefore defines a two-dimensional feature space, each dimension being discretized into 10 cells, each corresponding to a range of both components—this 10-by-10 space constitutes the MAP-Elite grid. In other implementations, the MAP-Elite grid may be formed by discretizing into a different number of cells.

In step 706, the variants scored in step 705 are input into the MAP-Elite grid. Each variant is input into the cell in the MAP-Elite grid that corresponds to its position in the T-SNE projection created in step 703. Following this, in step 707 a Pareto front is established in each cell. A Pareto front is a framework for filtering a set of solutions with multiple criteria—the criteria in this instance corresponding to the scores calculated previously, and is said to exist when all the solutions in a given set are in a non-dominance situation—i.e. each solution is superior, in at least one criteria, to all other solutions in that cell. Variants that form the Pareto front are retained in the MAP-Elite grid, while variants that do not are discarded.

In step 708 a batch of the retained variants from the MAP-Elite grid is sampled, and in step 709 the DNA sequence of each variant is copied. Mutations are then introduced into the DNA sequences, creating a mutated version of each variant. The mutated DNA sequences are translated into protein sequences.

In order for each mutated variant to be compared to the retained variants within the MAP-Elite grid, the mutated variants must be scored. To this end, in step 710 the mutated variants are input into the ensemble 302 of finely-tuned machine learning models, inference is performed as described previously with reference to FIG. 3, resulting in an output log-likelihood for each mutated variant from each model. Embeddings are also extracted from an intermediate layer of each model. This is performed in the same manner for the mutated variants as for the initial pool of variants in step 702.

Following the extraction of the embedding vectors for the mutated variants, in step 611 a dimensionality reduction is performed on the newly-extracted embedding vectors, to project the data points representing the mutated variants into the T-SNE space.

The process then repeats several of the steps described previously, namely steps 705 through 707. The mutated variants, having been projected into the T-SNE plot, are scored based on their log-likelihood (as calculated in step 710) and L1 distances from the centroid of each and every cluster within the T-SNE plot. The mutated variants are then input into the MAP-Elite grid, into the cell corresponding to their position on the T-SNE plot defined by the initial pool of variants. The scores assigned to each variant are analysed to determine whether the variant joins the Pareto front established for that cell. Depending on the scores of the mutated variant in question, the Pareto front in the cell may be unchanged. the mutated variant may join the Pareto front, or the mutated variant may displace one or more variants already in the cell.

This process is repeated until a predetermined number of variants have been considered. In other embodiments, it is possible to repeat this process until all valid mutations, that is all mutations that can be made whilst adhering to a relevant set of rules.

In one embodiment, such rules may include:
Restricting the mutations that may occur so that a specific ratio of transition (mutation of a nucleotide of a given type—purines (adenine and guanine) or pyrimidines (cytosine and thymine)—to a nucleotide of the same type (A<->G, C<->T)) to transversion (mutation of a nucleotide of a given type to a nucleotide of a different type—that is purine to pyrimidine or vice versa) is maintained.
Restricting the mutations to selected subparts of the sequence or so that mutations may not occur at neighbouring positions in the nucleic acid sequence. For example, mutations can be restricted to parts of the nucleic acid sequence encoding the receptor binding domain (RBD) and N-terminal domain (NTD) domains of the coronavirus spike protein.

Once the predetermined number of variants has been evaluated and the resulting Pareto fronts have been established in all the cells, the entire grid of retained variants may be analysed, in step 712. In this step, an overall Pareto front is established for the entire MAP-Elite grid—the scores of every retained variant are analysed to determine whether the variant in question forms the overall Pareto front, with those that do not being discarded. In step 713, the variants forming this overall Pareto front may be ranked, providing a list of the most likely candidates for an effective broad-spectrum vaccine.

The immunogenic compositions provided herein may, therefore, contain one or more immunogens which have been identified by the method as close to the centroid of one or more clusters of interest in a low dimensional projection. Indeed, in a scenario with multiple clusters of interest, the immunogenic composition may include several immunogens representing the centroids of each cluster of interest.

In a further embodiment, steps 708 to 711 may be omitted. In this embodiment, the method establishes a Pareto front based on the variants in the initial pool of variants 701, but the steps of introducing mutations (steps 708 to 711) are not performed. The result in step S713 is a ranking of the variants in the initial pool 701 to identify which of the variants from the initial pool are most likely candidates for an effective broad-spectrum vaccine.

In another embodiment, the scores of interest 2 to 4 above are not calculated to a centroid of the cluster, but are instead calculated to a variant of interest within a cluster. For example, the distances may be calculated between variants of interest in different clusters. For example, vaccine candidates could be selected to have a good response to the Beta and Delta variants of SARS-CoV-2. In such embodiments, the clustering steps can be omitted. This includes omitting the steps of performing T-SNE projection and the step of calculating the centre of the cluster.

As just noted, in the example above, the projection into T-SNE space is used to cluster the variants. However, in further embodiments, another method of clustering the multi-dimensional embedding vectors may be used. Accordingly, steps 703 and 711 may be omitted in some embodiments in which clustering is used.

### Examples

### Immunogen Used in Examples Below

For SARS-CoV-2, the primary focus for immunogens used in development of vaccines and antibody therapeutics has been the spike protein which is considered as the ideal target for COVID-19 immuinotherapies. This is based on previous evidence with SARS and MERS (Salvatori, et al. “SARS-CoV-2 SPIKE PROTEIN: an optimal inunological target for vaccines” 2020) and is also now being validated by the vaccines being used during the current pandemic.

The spike protein is a structural protein on the surface of SARS-CoV-2. An early study on recombinant vectors expressing the S protein of SARS-CoV found this protein to be highly immunogenic and protective against SARS-CoV challenge in hamsters, while in contrast, other surface proteins (the N, M, and E proteins) did not significantly contribute to a neutralizing antibody response or protective immunity (Buchholz, et al. 2004). Evidence of the key role played by the S protein in counteracting coronavirus infection came from studies on human-neutralizing antibodies from rare memory B cells of individuals infected with SARS-CoV (Traggiai, et al., “An efficient method to make human monoclonal antibodies from memory B cells: potent neutralization of SARS coronavirus” 2004) or MERS-Co (Corti, et al., “Prophylactic and postexposure efficacy of a potent human monoclonal antibod against MERS coronavirus” 2015). in such studies, antibodies directed against the S protein of SARS-CoV were found effective in inhibiting virus entry into the host cells. More recently, it has been found that SARS-CoV S elicited polyclonal antibody responses, and vigorously neutralized SARS-CoV-2 S-mediated entry into cells, thus further encouraging the use of this molecular target for vaccination and imununotherapies (Walls, et al_“Structure, Function, and Antigenicity of the SARS-CoV-2 Spike Glycoprotein” 2020).

Structural studies of antibodies in complex with SARS-CoV S and MERS-Coy S have provided information about the mechanism of competitive inhibition to the host receptor. The receptor-binding domain (RBD) in SARS-CoV-2 S protein was identified and found to bind strongly to ACE2 receptors. SARS-CoV RBD-specific antibodies cross-react with SARS-CoV-2 RBD protein, and SARS-CoV RED-induced antisera neutralized SARS-CoV-2, providing additional evidence that targeting this domain of the S protein of SARS-CoV-2 with a vaccine could be effective in preventive COVID-19. (Tai, et al., “Characterization of the receptor-binding domain (RBD) of 201) novel coronavirus: implication for development of RBD protein as a viral attachment inhibitor and vaccine” 2020).

### Example 1a

A detailed worked example concerns the COVID-19 crisis. During the spread of the virus, the situation was immensely complicated and rendered considerably more dangerous through the emergence of a myriad of variants of the ‘original’ virus. Adapting to the environment through mutagenesis, SARS-CoV-2 served as the starting point for many variations on a common theme. Most of these variants were less ‘fit’ than their progenitor, i.e. less virulent, less transmissible, less able to survive. However, certain notable variants were markedly more ‘fit,’ being more virulent, more transmissible, and, importantly, less susceptible to detection by the human immune system.

Using methods known in the art, it is very difficult to determine at the outset whether a newly-observed variant has the propensity to increase its ‘fitness’ to a point that it will become more concerning—i.e. whether this variant is likely to pose a danger to humans and society. For instance, variant B.1.1.7 (the ‘UK variant’) was sequenced and identified in September 2020, but was not recognized as a variant of concern until December 2020. A similar delay was observed in relation to both the B.1.351 (‘South African variant’) and P.1 (‘Brazilian variant’) variants. This lag in recognizing the danger of newly-sequenced variants is a factor in tens, conceivably hundreds, of thousands of deaths. Embodiments provide the ability to reduce this delay, as will be shown in the following example.

In this example, the method of identifying and ranking existing variants of a pathogen is applied to the SARS-CoV-2 virus. The results provided below demonstrate that had this method been available and utilised at the point of initial observation and sequencing of these variants, they would have been ranked and categorized as variants of concern far sooner than by the methods currently used in the art.

To begin, data was collected from the GISAID database, with all data thus collected having been initially gathered prior to (and not including) 20 Sep. 2020. Potentially mislabelled data (such as samples of SARS-CoV-2 listed as being gathered prior to December 2019) was excluded from the training dataset, so as to eliminate data that could bias the experiment. No UK (B.1.1.7), South African (B.1.351) or Brazilian (P.1) sequences were observed in this training data. Two sequences of 242,488 in the training set were observed to have partial UK (B.1.1.7) characteristics, having some but not all (<50%) of the characteristic mutations.

The model was trained as described previously on this training data. The trained model was then used to evaluate an initial pool of variants as sampled on 20 Sep. 2020. The pool of variants was then updated iteratively, with each update containing the sequences sampled as of the end of each month. The initial pool thus contained all relevant samples up to 20 Sep. 2020, the first update added samples up to the end of October 2020, the second to the end of November 2020, and so on. It should be stressed that the model was at no point re-trained on the new sequences—the new variants were added to the pool of variants for evaluation only.

The following violin plots show the results provided from this evaluation. The evaluation of the background dataset (i.e. the samples from all types of the virus) is represented as ‘Other,’ and provides a median against which to measure the specific variants listed.

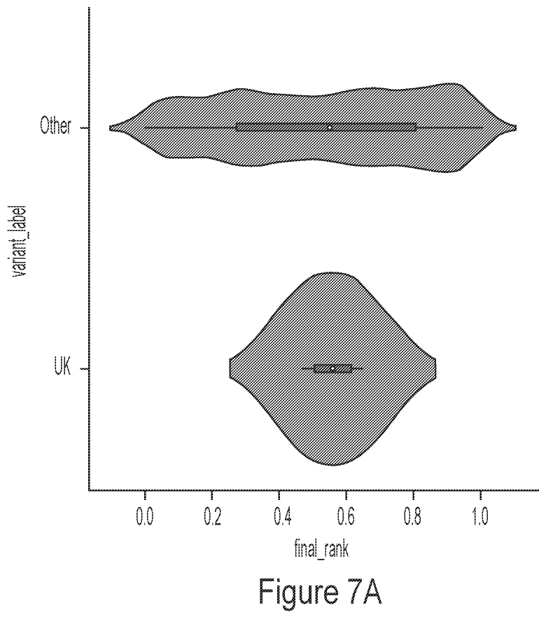

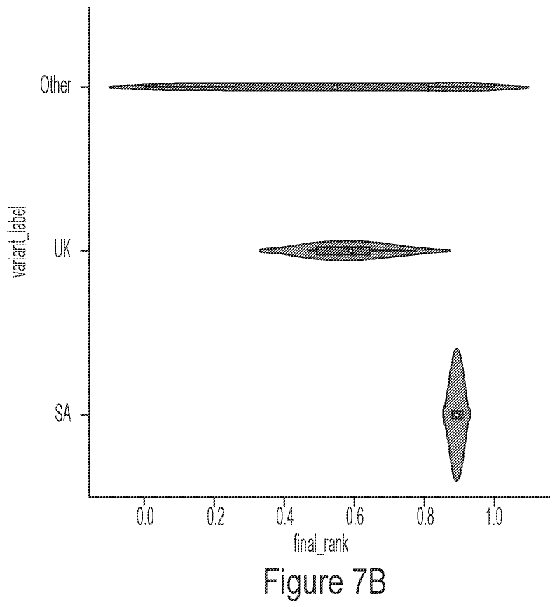

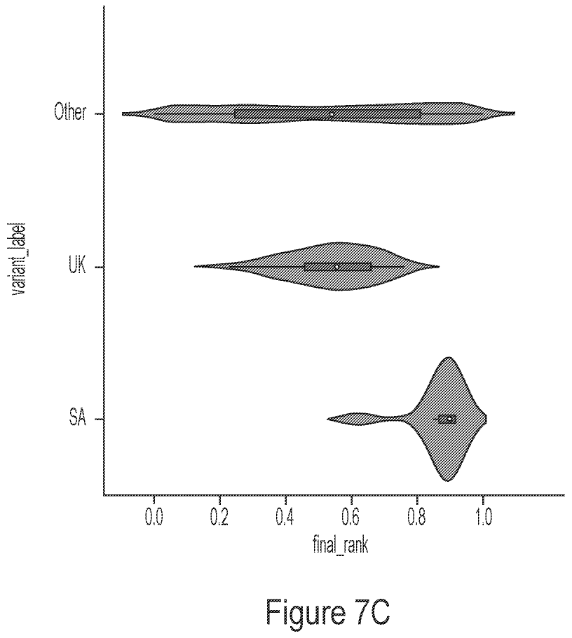

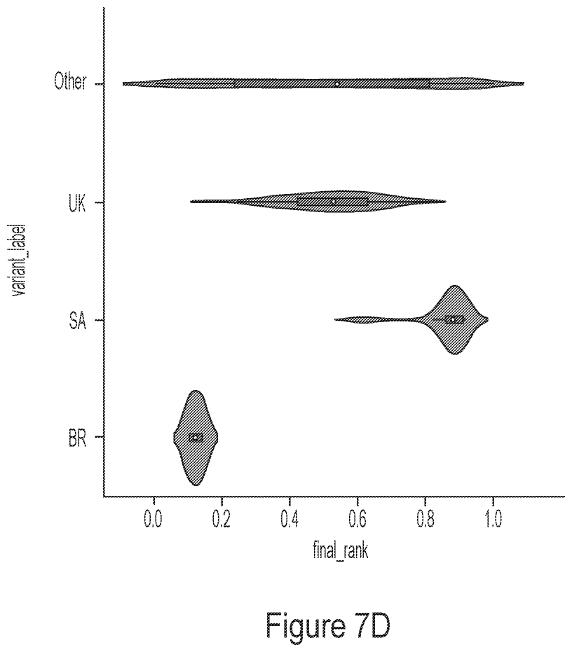

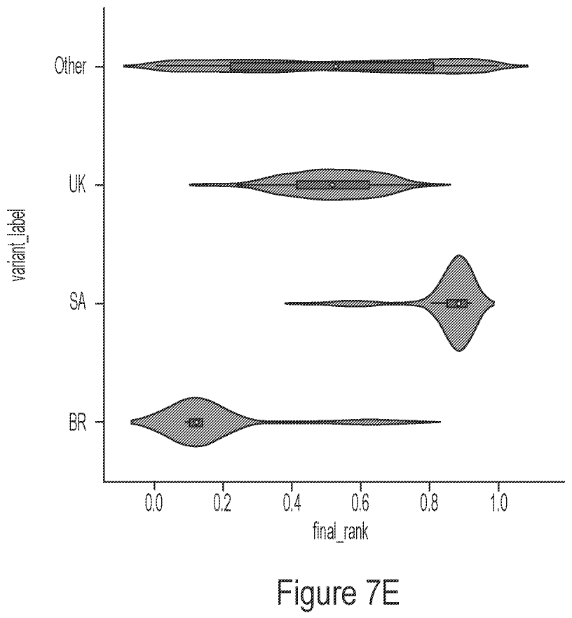

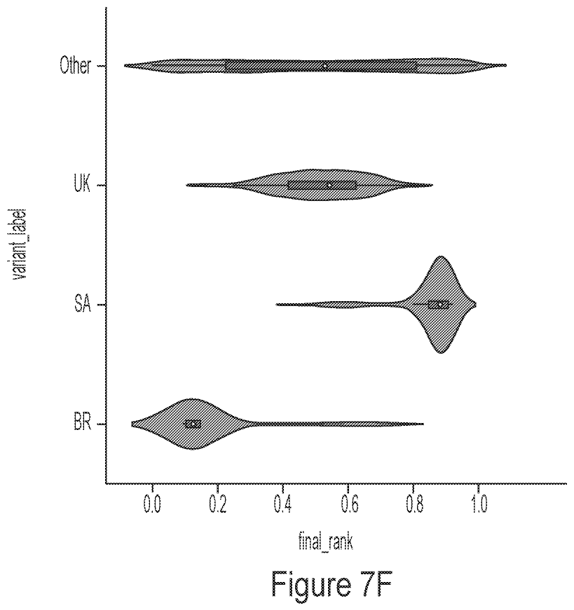

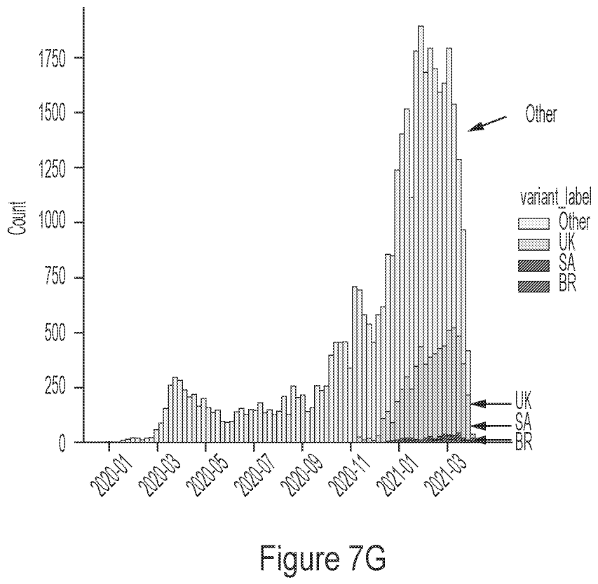

FIG. 7A shows the UK (B.1.1.7) variant ranked against the median. As can be seen, it was ranked as more dangerous, but only slightly. This is to be expected—while the initial version of the UK variant was observed on 20 Sep. 2020, the variant increased significantly in danger with the emergence of later sub-variants, more competent than the initial variant. The low representation of variants such as the UK (B.1.1.7) variant in the training data pool caused the model to initially classify it as putatively dangerous, but potentially too far removed from the observed sequences to be properly ‘fit.’Variants exhibiting more indels (insertions/deletions), such as the UK (B.1.1.7) variant, increased in abundance in the source sequence database after 20 Sep. 2020; had the model been retrained on updated datasets including such variants, the relative importance level of the UK (B.1.1.7) variant would have increased dramatically as the model would have been able to recognize it more effectively. The skilled person will appreciate that this is a function of the methodology applied in this example, rather than of the method itself.

As new, slightly more competent variants were sequenced and appeared in the pool of variants for evaluation, the rankings shifted accordingly. The results from the evaluation of samples as of the end of October, shown in FIG. 7B, show that the newly-observed South African (B.1.351) variant was immediately ranked as a variant of concern, being evaluated by the algorithm as highly ‘fit’ and likely to escape the immune response. Additionally, as more sub-variants of the UK (B.1.1.7) variant were added to the pool, the overall ranking for that variant increased.

The trend from the first two plots is continued in subsequent months. In the sequences gathered as of the end of November, the evaluation of which is shown is FIG. 7C, a greater number of sub-variants were sampled for both the UK (B.1.1.7) and South African (B.1.351) variants. As a natural consequence of this, the data shows the emergence of strains with lower ‘fitness’ and correspondingly lower scores. However, due to the evolutionary advantage granted by the ‘core’ variants, they remain feasible.

Furthermore, while the average rank for the UK (B.1.1.7) variant appears only medium, it should be stressed that the ranking is comparative. Therefore, the rank assigned to the UK variant would still be entirely sufficient to categorize this variant as a variant of concern. Furthermore, the plot shows that sub-variants of this type are still ranked highly. It is therefore apparent that the UK (B.1.1.7) variant has a clear evolutionary path to become significantly more dangerous, solidifying its status as a variant of concern.

In December, shown in FIG. 7D, the Brazilian (P.1) variant entered the pool of variants for the first time. At the outset, this variant is ranked as being of little concern. As will be shown in subsequent plots and discussed below, this evaluation is entirely accurate based on the variants then in the pool. The South African (B.1.351) variant remains consistently highly ranked, marking it as a variant of significant concern.

FIG. 7E, showing the results of an evaluation of a pool of variants sequenced as of the end of January 2021, demonstrates the evolution of a variant with a low ranking into a variant of concern. Newly-sequenced sub-variants of the Brazilian (P.1) variant entered the pool at this point, and were immediately given a high ranking. This suggests that the Brazilian (P.1) variant has an evolutionary path to become a variant of concern.

At this point it must be reiterated that the model performing the evaluation resulting in FIG. 7E was trained on data parameterized to 20 Sep. 2020, meaning that up to this point, the model had not been provided with examples of the South African (B.1.351) or Brazilian (P.1) variants, yet still ranked them as variants of concern.

These results strongly suggest that had the methods described herein been available and utilized at the point at which these variants were observed and sequenced, it would have identified and labelled these mutations as dangerous ab initio, saving time and therefore lives.

FIG. 7F shows the results as of mid-April 2021. The UK (B.1.1.7) variant appears similar to the background distribution, which is still sufficient to mark it as a variant of concern. The South African (B.1.351) and Brazilian (P.1) variants demonstrate notably high evolutionary fitness.

These results are in accord with published vaccine-elicited resistance data across variants; SARS-CoV-2 variants B1.1.7, B.1.351 and P.1 have been classified as Variants of Concern by several health authorities, indicating that there is evidence that a) these variants impact diagnostics, treatments or vaccines; b) increased transmissibility and c) increased disease severity.

This confirms the accuracy of the model, even when trained only on data sampled prior to the appearance of the variants ranked.

The bar chart shown in FIG. 7G illustrates the number of sequences relating to each variant sampled over time. This is provided to demonstrate that scores are not artificially inflated due to a greater number of sequences from a particular variant being sampled.

From the results shown in FIGS. 7A through 7F, it can be seen that when the method was applied to SARS-CoV-2 and trained on sequences of that virus, it was able to identify and rank the UK (B.1.1.7), South African (B.1.351) and Brazilian (P.1) variants of the virus, and indeed sub-variants thereof, as dangerous as soon as they were entered into the pool of variants for evaluation. This was the case despite the method having been trained exclusively on data gathered prior to the emergence and sequencing of these variants. In other words, without any prior exposure to these variants, the method immediately identified the most dangerous sub-variants thereof and ranked them accordingly

From this, it follows that this method would have been able to mark the UK (B.1.1.7), South African (B.1.351) and Brazilian (P.1) variants as variants of concern at the point at which they were first observed and sequenced, had it been available at the time.

This is a marked improvement over the state of the art—as stated, it took several months for methods currently used in the art to make this determination. The method described above would therefore reduce the delay between a new variant being observed and recognition of it as dangerous. This reduction in delay could represent an immense reduction in lives lost.

### Example 1b

The example provided above presumes a method according to the first embodiment, wherein only the scores specifically stated are utilised. However, as noted in connection with FIG. 3, additional scores may also be used to determine the overall ranking of a particular variant.

This example provides a demonstration of the results of including, as an additional score, a knowledge-based criterion. The results show that by doing so, dangerous variants can be identified even faster and with even greater clarity than when relying solely on the data-driven metrics specified.

This example utilises, as the additional score, the score described above in connection with a further embodiment, specifically a score related to the epitope. To obtain this score, each amino acid position in the protein sequence of the variant being evaluated was assessed a further score, calculated as a combination of surface availability, known antibody epitopes, and structural proximity to these known epitopes. This score requires experimental data to calculate, as it may be found through statistical bioinformatics and structure means. The data used for construction of this metric has been openly and freely available before Sep. 20, 2020. Construction of the metric was done by purely automated, data-driven means and required no expert input, making it robust, data-driven and directly transferable to other tasks of this nature.

In all other aspects, the results from Example 1b were obtained through the same process as Example 1a, above. The model used was trained on the same data gathered prior to 20 Sep. 2020, then used to evaluate an initial pool of variants as sampled on 20 Sep. 2020. The pool of variants was then updated iteratively, with each update containing the sequences sampled as of the end of each month.

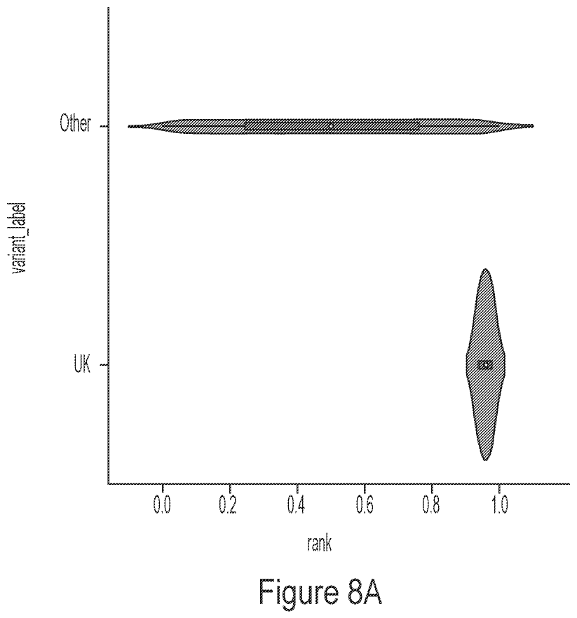

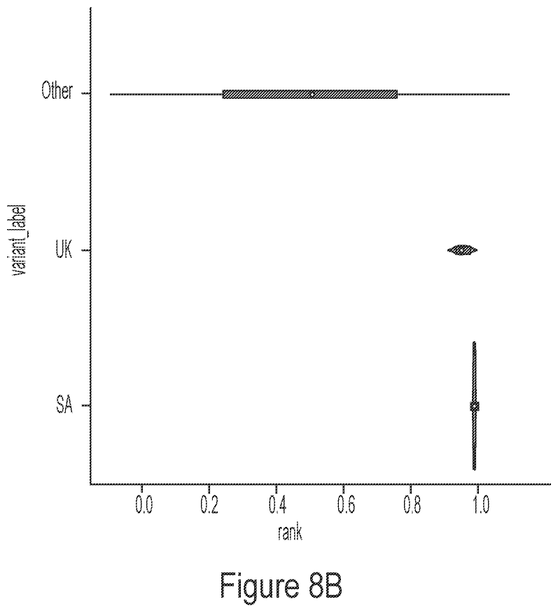

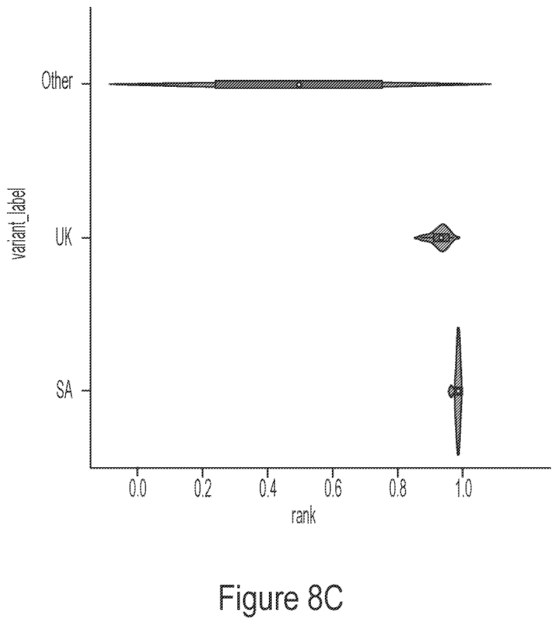

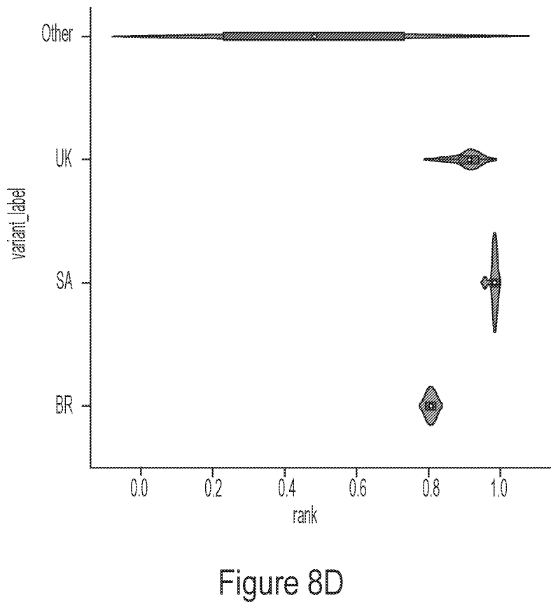

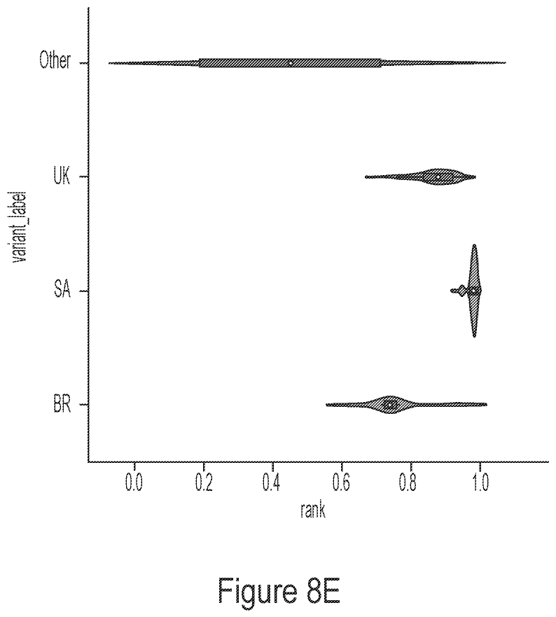

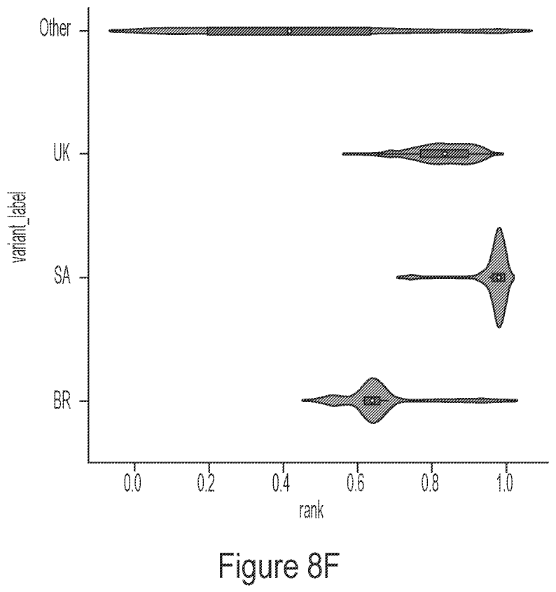

FIG. 8A shows the results of an evaluation of the data as of 20 Sep. 2020, taking into account the additional score. The UK (B.1.1.7) variant has, as a characteristic, mutations in the antibody binding sites. It consequently received a very high additional score, meaning that it was marked as highly dangerous as soon as it entered the pool of variants to be evaluated.

The South African (B.1.351) variant entered the pool of variants for evaluation in October, as noted above. FIG. 8B shows that it is assigned a very high ranking immediately, while FIG. 8C illustrates that this ranking is sustained through November. As this was the case without the additional score, the difference here is less notable than that shown in connection with FIG. 8A.

FIG. 8D, showing the results of the evaluation on variants sampled as of the end of December 2020, shows the effects of the additional score on the ranking of the newly-sequenced Brazilian (P.1) variant. Similar to the UK (B.1.1.7) variant, the Brazilian (P.1) variant displays characteristic mutations in the antibody binding sites, resulting in a high additional score. Consequently, the Brazilian (P.1) variant is immediately ranked as highly dangerous.

FIGS. 8A and 8D together demonstrate the impact this additional score can have on the ranking of a variant. Both the UK (B.1.1.7) and Brazilian (P.1) variants are ranked as substantially more dangerous when this score is taken into account, being immediately identified as variants of concern.

This demonstrates the advantages offered by the method described above. Even when using a model trained on data that, at the point of evaluation, was almost half a year out of date, the method was able to accurately evaluate variants as particularly dangerous immediately on their introduction to the pool of variants for evaluation.

From the results presented above, it is apparent that had the method, as used in this example, been available and utilized to evaluate SARS-CoV-2 variants in September 2020, the UK (B.1.1.7) variant would have been immediately categorised as a variant of concern, allowing for a faster and more comprehensive response to the danger posed. Furthermore, the South African (B.1.351) and Brazilian (P.1) variants would have been categorised as variants of concern immediately upon their observation and sequencing, rather than months thereafter. The reduction in delay could result in a reduction in the number of deaths.

FIG. 8E shows the evaluation of a pool of variants sampled through the end of January 2021. As may be seen, the Brazilian (P.1) variant has increased in ranking, confirming its identification as a variant of concern in December 2020, shown in FIG. 8D. It should be noted that in reality, this same variant was only identified as dangerous on 21 Jan. 2021; this method would therefore have been able to identify the variant as dangerous as soon as it was sequenced, at least a month faster than methods used in reality, even accounting for training data that would have been, at the time of identification, three months out of date.

FIG. 8F shows the results of the evaluation of data gathered up to the present; all sequenced variants have been evaluated by the method and scored according to both the original criteria of the first embodiment and the further criterion relating to the epitope location. As may be seen, while the results are similar to those shown in Example 1a, specifically FIG. 7F, the method in this example has accurately increased the rankings of known variants of concern.

### Example 2—Vaccine Design

The fundamental goal of a vaccine is to elicit an immune response to a given immunogen. A further worked example concerns the SARS-CoV-2 virus, and demonstrates the way in which the method proposes candidate vaccines.

Current vaccines largely train the immune system to recognize the wild type strain of SARS-CoV-2. However, as discussed previously the virus is liable to mutate and adapt; some of these mutations occur at the positions in the regions recognized by the immune system (epitopes)—such mutations increase the risk that the mutant variant will evade the immune response elicited by a vaccine targeted on the wild type.

Studies in the field have identified nine epitopes to which antibodies elicited by the SARS-CoV-2 virus, or vaccination for the same, are likely to bind. These epitopes are listed in the table below.

| Epitope | Residues |
|---|---|
| E1 | 15-28, 63-79, 247-260 |
| E2 | 97, 178-189, 207-219 |
| E3 | 137-164 |
| E4 | 332-346 |
| E5 | 403-406, 438, 440-451, 495-506 |
| E6 | 452-476, 479-482, 484-494 |
| E7 | 527-537 |
| E8 | 603-605, 633-642, 656-661, 674-693 |
| E9 | 808-814 |

As stated, a mutation in any of these regions is likely to affect the immune response, as antibodies produced in response to a vaccine presenting a corresponding epitope will no longer bind to a variant with this mutation as specifically as to the wild type. Certain epitopes (like E4 or E5) are located in the Receptor Binding Domain (RBD) region of the spike protein, a prime target for the generation of antibodies. A large fraction of antibodies sequenced from convalescent sera target RBD epitopes.

An ideal vaccine candidate would therefore prime the immune system to recognize multiple epitopes, as well as mutated variants thereof. However, the number of mutations that can be contained within the protein sequence of a vaccine candidate are limited—if too many mutations are introduced, the sequence will no longer resemble the original version of the immunogen.

In this example, a comprehensive set of 4700 SARS-CoV-2 variants, representing the most common variants of the spike protein, were used as starting points for the vaccine design process. These were used to train a language model as described in connection with FIGS. 1 and 2; the language model of this example used Facebook AI Research ESM Transformer architecture. The trained model was then fine-tuned on the entire, identity-reduced GISAID dataset of spike proteins, as described in connection with FIGS. 1 and 2. The vaccine design process was performed over 24 hours, using a computing architecture of 8 NVIDIA A100 GPUs and 40 CPU cores. When introducing mutations as described in connection with FIG. 6B, only the N-terminal domain (NTD) and RBD were targeted, as these are the regions deemed most relevant for the immune escape potential of SARS-CoV-2 variants. These mutations were guided by the transition probabilities described in connection with FIG. 6B, as well as a freely chosen prior probability of 1% to introduce mutations. The inference and evaluation process described in FIG. 6 was carried out, with relevant embedding vectors being extracted and projected in T-SNE space. An arbitrary number of cluster centres were selected, in this case two, which were computed using the entire GISAID dataset. Three criteria were therefore used to score each variant: L1 distance to cluster centre 1, L1 distance to cluster centre 2, and a log-likelihood with regard to the underlying ESM model. It should be noted that while the clusters were computed using all the data, the initial pool of variants for redesign contained only 4700 data points, slightly more than 10% of the full data set (over 44,000 unique sequences).

After scoring the variants using the criteria discussed, and entering them into an optimization algorithm as described in connection with FIG. 6, the remaining designs were ranked, and the top 25% of the newly designed vaccines were sampled. This yielded 673 candidates. The same number of unique sequences from the GISAID database were also sampled, to provide an estimate of the current mutational landscape of the virus.

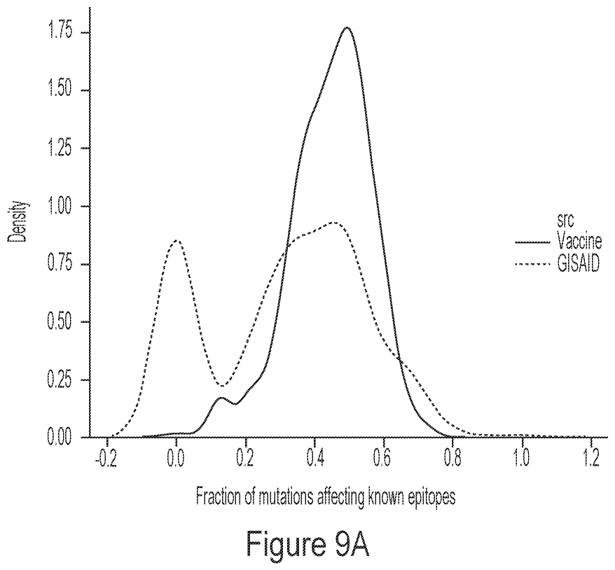

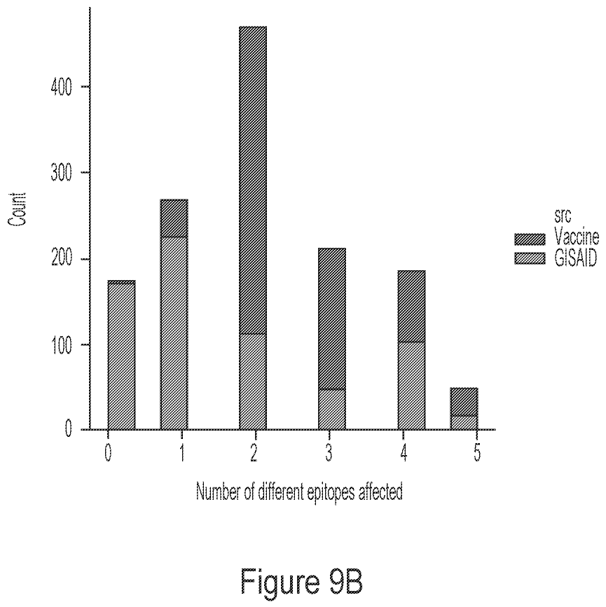

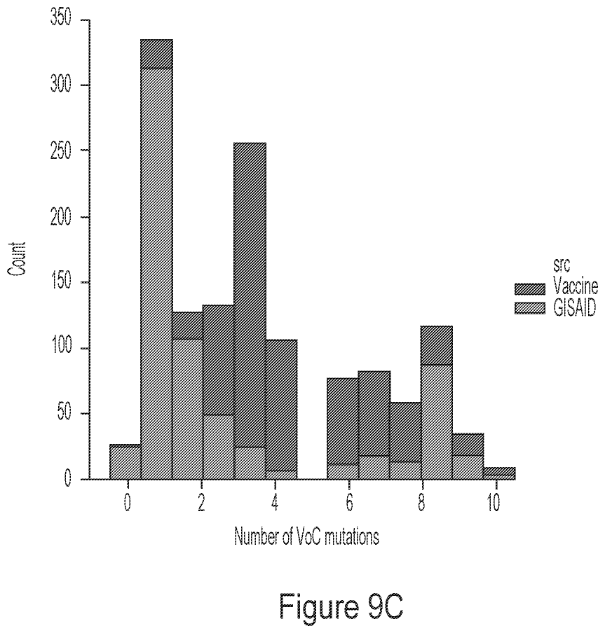

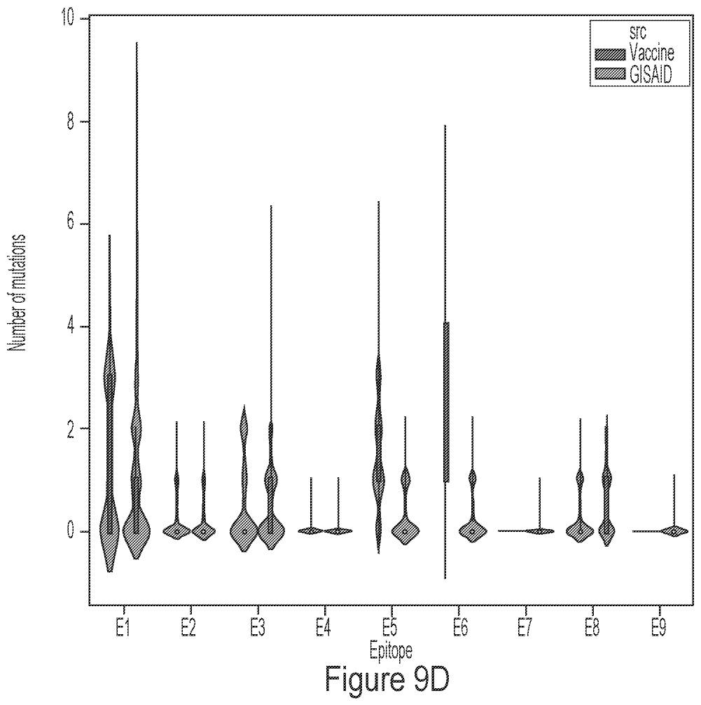

FIG. 9A shows a comparison of the number of variants in each pool (the vaccine candidates and the GISAID randomly-sampled variants) for which a particular fraction of their mutations affected known epitopes. It should be stressed at this point that the model used in this example had not been trained on epitope-specific data—i.e. it had not been ‘taught’ to target variants having mutations affecting known epitopes.

In FIG. 9A, it can be seen that all vaccine sequences proposed by the method affected known epitopes to a greater or lesser degree. However, it may also be observed that no more than 70% of the mutations in the vaccine candidate variants are specifically directed towards known antibody epitopes. This can be attributed to several factors:

First, the method used circulating virus variants as a starting point. This would limit the number of permissible mutations to epitopes of a given variant, before the variant would lose ‘fitness’ and suffer a drop in the log-likelihood score, as it would no longer resemble a naturally-occurring variant.

Second, the functional constraints of the nature of the spike protein, especially compensating mutations. If too many mutations in a given variant are directed towards antibody epitopes, the overall spike protein will lose coherence.

Third, it is quite likely that mutations in the vaccine candidates target not only known antibody epitopes, but also potential T-cell epitopes. As discussed above, this makes it likely that vaccine variants produced by this method impact not only antibody-related but also T-cell related virus immunity.

FIG. 9B is a chart showing the number of epitopes affected by the mutations in both pools of variants. The results are as expected, with the randomly-sampled variants showing a broad distribution of epitopes affected, while the vaccine candidates affect a far narrower ranger. This can be attributed to the fundamental purpose of the vaccine. A vaccine that addresses zero epitopes would not make a difference from the immune point of view, while one that addresses all of them (in this case—nine), would be useless, as it would remove all the defining characteristics of the virus—it would no longer bear a resemblance to the naturally-occurring variant.

FIG. 9C illustrates the number of variants from each pool of variants that contain specific mutations relating to known variants of concern. Many currently circulating strains evade the immune system to a lesser or greater extent, with mutations such as L452R, E484K/Q, K417N and N501Y becoming increasingly more prevalent. Therefore, it is of paramount importance for the vaccine candidates to elicit an immune response against Variant of Concern mutations, to closely mimic the real-world situation. However, even when only taking into account three variants officially named as Variants of Concern—the UK (B.1.1.7) variant, South African (B.1.351) variant and Brazilian (P.1) variant—yields 31 unique mutations, for too many to include in a single design and still maintain internal consistency.

From FIG. 9C, it may be observed that, in comparison with the circulating strains, vaccine sequences produced as described comprise a greater number of Variant of Concern mutations, and as such putatively form better stimuli for the immune system than any of the wild type strains. It should be reiterated at this point that the method as used in this example is not aware of Variants of Concern, and as such does not explicitly aim to maximise the number such mutations.

FIG. 9D illustrates the number of variants in each pool of variants having mutations affecting specific epitopes. As a limited number of mutations are possible in each variant sequence, with higher number of mutations being strongly associated with a diminished stability, lowered similarity to target pathogen, and protein translation issues, this example demonstrates how a targeted design process as described would result in a sufficient number of mutations per epitope, than a high number of mutations overall. Conversely, one would aim to maximize the number of affected epitopes, to cover the broadest possible range of mutations while maintaining internal consistency.

In FIG. 9D, such a distribution of mutations over the known epitopes may be observed. The vaccine candidates display no mutations in epitopes E7 and E9, which is a direct consequence of these epitopes not being in RBD or NTD, which as discussed previously means that mutations here would not particularly impact immune escape potential. Furthermore, the vaccine candidate variants have a low number of E8 mutations, where all the mutations observed have originated from the initial starting variants.

However, the vaccine candidate variants display a far greater number of mutations in epitopes E5 and E6, regions in the RBD, than do the randomly-sampled GISAID variants. Mutations here present a vastly different E5/E6 binding interface, leading to production of other classes of antibodies—some of which recognize the neo-E5/E6 interface, but many of which aim at other epitopes as well. This demonstrates the ability of the method described to identify regions in which mutations will most significantly impact the immune escape potential of SARS-CoV-2 variants, despite not having been trained on epitope-specific data.

It may also be observed, from FIG. 9D, that the vaccine candidate variants display a far lower number of E3 mutations than the randomly-sampled pool. This may be attributed to the majority of E3 mutations in circulating variants being deletions, which lead vastly different epitopes, lowering the similarity of the variant in question to the ‘original’ wild-type variants. By exercising a limited number of E3 mutations, the vaccine candidate variants designed as describe predictably change this epitope, but have sufficient mutation space to focus on other regions.

The protein sequences of the top 10 vaccine candidate variants generated as described are provided below. These vaccine candidate variants are all based on the B.1.1.7 scaffold, which is fully justified, considering that 80% of Covid-19 cases worldwide reported at the time of this process being conducted were due to viruses of this lineage. These designs include additional, yet unseen variations in the potentially immunogenic regions, coupled with novel combinations of mutations from the Variants of Concern.

### Sample Sequences of 10 Top Ranked Vaccines

SEQ ID NO. 1
>vaccine01|L5P L7S S13N K386E D389N K417N S477N E484K S494P N501Y
MFVFPVSLPLVSNQCVNLTTRTQLPPAYTNSFTRGVYYPDKVFRSSVLHSTQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKN
IDGYFKIYSKHTPINLVRDLPQGFSALEPLVDLPIGINITRFQTLLALHRSYLTPGDSSSGWTAGAAAYYVGYLQPRTFLLKYNENGTITDAVDCALD
PLSETKCTLKSFTVEKGIYQTSNERVQPTESIVRFPNITNLCPFGEVFNATRFASVYAWNRKRISNCVADYSVLYNSASFSTFKCYGVSPTELNNLCF
TNVYADSFVIRGDEVRQIAPGQTGNIADYNYKLPDDFTGCVIAWNSNNLDSKVGGNYNYLYRLFRKSNLKPFERDISTEIYQAGNTPCNGVKGFNCYF
PLQPYGFQPTYGVGYQPYRVVVLSFELLHAPATVCGPKKSTNLVKNKCVNFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARS
VASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGKIQDSLSSTASALGKLQDVVNQNAQALNTLVKQLSSNFGAISSVLNDI
LSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT
SEQ ID NO. 2
>vaccine02|L5P L7S S13N D80A K386E K417N S477N E484K S494P N501Y
MFVFPVSLPLVSNQCVNLTTRTQLPPAYTNSFTRGVYYPDKVFRSSVLHSTQDLFLPFFSNVTWFHAIHVSGTNGTKRFANPVLPFNDGVYFASTEKS
NIIRGWIFGTTLDSKTQSLLIVNNATNVVIKVCEFQFCNDPFLGVYYHKNNKSWMESEFRVYSSANNCTFEYVSQPFLMDLEGKQGNEKNLREFVFKN
IDGYFKIYSKHTPINLVRDLPQGFSALEPLVDLPIGINITRFQTLLALHRSYLTPGDSSSGWTAGAAAYYVGYLQPRTFLLKYNENGTITDAVDCALD
PLSETKCTLKSFTVEKGIYQTSNERVQPTESIVRFPNITNLCPFGEVFNATRFASVYAWNRKRISNCVADYSVLYNSASFSTFKCYGVSPTELNDLCF
TNVYADSFVIRGDEVRQIAPGQTGNIADYNYKLPDDFTGCVIAWNSNNLDSKVGGNYNYLYRLFRKSNLKPFERDISTEIYQAGNTPCNGVKGFNCYF
PLQPYGFQPTYGVGYQPYRVVVLSFELLHAPATVCGPKKSTNLVKNKCVNFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARS
VASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQ
VKQIYKTPPIKDFGGFNFSQILPDPSKPSKRSFIEDLLENKVTLADAGFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGKIQDSLSSTASALGKLQDVVNQNAQALNTLVKQLSSNFGAISSVLNDI
LSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT
SEQ ID NO. 3
>vaccine03|F4S L5P L7S S12P S13N V120I D215G C291-S477N
MFVSPVSLPLVPNQCVNLTTRTQLPPAYTNSFTRGVYYPDKVFRSSVLHSTQDLFLPFFSNVTWFHAIHVSGTNGTKRFDNPVLPFNDGVYFASTEKS
NIIRGWIFGTTLDSKTQSLLIINNATNVVIKVCEFQFCNDPFLGVYYHKNNKSWMESEFRVYSSANNCTFEYVSQPFLMDLEGKQGNFKNLREFVFKN
IDGYFKIYSKHTPINLVRGLPQGFSALEPLVDLPIGINITRFQTLLALHRSYLTPGDSSSGWTAGAAAYYVGYLQPRTFLLKYNENGTITDAVDALDP
LSETKCTLKSFTVEKGIYQTSNFRVQPTESIVRFPNITNLCPFGEVFNATRFASVYAWNRKRISNCVADYSVLYNSASFSTFKCYGVSPTKLNDLCFT
NVYADSFVIRGDEVRQIAPGQTGKIADYNYKLPDDFTGCVIAWNSNNLDSKVGGNYNYLYRLFRKSNLKPFFRDISTEIYQAGNTPCNGVEGFNCYFP
LQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITP
CSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSV
ASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDVVIGIVNNTVYDPLQPELDSFKEELDKYFKNHTSPDVDLGDISGINASVV
NIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT
SEQ ID NO. 4
>vaccine04|L5R L7S S13N P26S F55L H69-Y145-S221-A372V K417N S477N N501Y
MFVFRVSLPLVSNQCVNLTTRTQLPSAYTNSFTRGVYYPDKVFRSSVLHSTQDLLLPFFSNVTWFHAIVSGTNGTKRFDNPVLPFNDGVYFASTEKSN
IIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNID
GYFKIYSKHTPINLVRDLPQGFALEPLVDLPIGINITRFQTLLALHRSYLTPGDSSSGWTAGAAAYYVGYLQPRTFLLKYNENGTITDAVDCALDPLS
ETKCTLKSFTVEKGIYQTSNERVQPTESIVRFPNITNLCPFGEVFNATRFASVYAWNRKRISNCVADYSVLYNSVSFSTFKCYGVSPTKLNDLCFTNV
YADSFVIRGDEVRQIAPGQTGNIADYNYKLPDDFTGCVIAWNSNNLDSKVGGNYNYLYRLERKSNLKPFERDISTEIYQAGNTPCNGVEGFNCYFPLQ
SYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCS
FGGVSVITPGTNTSNQVAVLYQDVNCTEVPVAIHADQLTPTWRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQDSLSSTASALGKLQDVVNQNAQALNTLVKQLSSNFGAISSVLNDILSR
LDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT
SEQ ID NO. 5
>vaccine05|L5P L7S S13N P26S D80A T240- L241- L242- T323I K417N R457G K458R E484K N501Y
MFVFPVSLPLVSNQCVNLTTRTQLPSAYTNSFTRGVYYPDKVFRSSVLHSTQDLFLPFFSNVTWFHAIHVSGTNGTKRFANPVLPFNDGVYFASTEKS
NIIRGWIFGTTLDSKTQSLLIVNNATNVVIKVCEFQFCNDPFLGVYYHKNNKSWMESEFRVYSSANNCTFEYVSQPFLMDLEGKQGNEKNLREFVFKN
IDGYFKIYSKHTPINLVRDLPQGFSALEPLVDLPIGINITRFQALHRSYLTPGDSSSGWTAGAAAYYVGYLQPRTFLLKYNENGTITDAVDCALDPLS
ETKCTLKSFTVEKGIYQTSNERVQPIESIVRFPNITNLCPFGEVFNATRFASVYAWNRKRISNCVADYSVLYNSASFSTFKCYGVSPTKLNDLCFTNV
YADSFVIRGDEVRQIAPGQTGNIADYNYKLPDDFTGCVIAWNSNNLDSKVGGNYNYLYRLFGRSNLKPFERDISTEIYQAGSTPCNGVKGFNCYFPLQ
SYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCS
FGGVSVITPGTNTSNQVAVLYQDVNCTEVPVAIHADQLTPTWRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQ
IYKTPPIKDFGGFNFSQILPDPSKPSKRSFIEDLLENKVTLADAGFIKQYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQDSLSSTASALGKLQDVVNQNAQALNTLVKQLSSNFGAISSVLNDILSR
LDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDPLQPELDSFKEELDKYFKNHTSPDVDLGDISGINASVVNI
QKEIDRINEVAKNLNESLIDLQELGKYEQYIKWPWYIWLGFIAGLIAIVMVTIMLCCMTSCCSCLKGCCSCGSCCKFDEDDSEPVLKGVKLHYT
SEQ ID NO. 6
>vaccine06|L5P L7S S13N V327I K386E K417N S477N E484K S494P N501Y
MFVFPVSLPLVSNQCVNLTTRTQLPPAYTNSFTRGVYYPDKVFRSSVLHSTQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHKNNKSWMESEFRVYSSANNCTFEYVSQPFLMDLEGKQGNEKNLREFVFKN
IDGYFKIYSKHTPINLVRDLPQGFSALEPLVDLPIGINITRFQTLLALHRSYLTPGDSSSGWTAGAAAYYVGYLQPRTFLLKYNENGTITDAVDCALD
PLSETKCTLKSFTVEKGIYQTSNERVQPTESIIRFPNITNLCPFGEVFNATRFASVYAWNRKRISNCVADYSVLYNSASFSTFKCYGVSPTELNDLCF
TNVYADSFVIRGDEVRQIAPGQTGNIADYNYKLPDDFTGCVIAWNSNNLDSKVGGNYNYLYRLFRKSNLKPFERDISTEIYQAGNTPCNGVKGFNCYF
PLQPYGFQPTYGVGYQPYRVVVLSFELLHAPATVCGPKKSTNLVKNKCVNFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARS
VASQSIIAYTMSIGAENSVAYSNNSIAIPTNFTISVTTEILPVSMTKTSVDCTMYICGDSTECSNLLLQYGSFCTQLNRALTGIAVEQDKNTQEVFAQ
VKQIYKTPPIKDFGGFNFSQILPDPSKPSKRSFIEDLLENKVTLADAGFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGKIQDSLSSTASALGKLQDVVNQNAQALNTLVKQLSSNFGAISSVLNDI
LSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT
SEQ ID NO. 7
>vaccine07|L5P L7S S13N T240-L242-K417N R457G E484K T500I N501Y
MFVFPVSLPLVSNQCVNLTTRTQLPPAYTNSFTRGVYYPDKVFRSSVLHSTQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHKNNKSWMESEFRVYSSANNCTFEYVSQPFLMDLEGKQGNEKNLREFVFKN
IDGYFKIYSKHTPINLVRDLPQGFSALEPLVDLPIGINITRFQLALHRSYLTPGDSSSGWTAGAAAYYVGYLQPRTFLLKYNENGTITDAVDCALDPL
SETKCTLKSFTVEKGIYQTSNERVQPTESIVRFPNITNLCPFGEVFNATRFASVYAWNRKRISNCVADYSVLYNSASFSTFKCYGVSPTKLNDLCFTN
VYADSFVIRGDEVRQIAPGQTGNIADYNYKLPDDFTGCVIAWNSNNLDSKVGGNYNYLYRLFGKSNLKPFERDISTEIYQAGSTPCNGVKGFNCYFPL
QSYGFQPIYGVGYQPYRVVVLSFELLHAPATVCGPKKSTNLVKNKCVNFNFNGLTGTGVLTESNKKFLPFQQFGRDIADTTDAVRDPQTLEILDITPC
SFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVA
SQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVK
QIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITS
GWTFGAGAALQIPFAMQMAYRFNGIGVTQNVLYENQKLIANQFNSAIGKIQDSLSSTASALGKLQDVVNQNAQALNTLVKQLSSNFGAISSVLNDILS
RLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDPLQPELDSFKEELDKYFKNHTSPDVDLGDISGINASVVN
IQKEIDRLNEVAKNINESLIDLQELGKYEQYIKWPWYIWLGFIAGLIAIVMVTIMLCCMTSCCSCLKGCCSCGSCCKFDEDDSEPVLKGVKLHYT
SEQ ID NO. 8
>vaccine08|L5R L7S S13N H69-V70-Y145-1472M E484K N501Y A570D P681H D1118H K1191N
MFVFRVSLPLVSNQCVNLTTRTQLPPAYTNSFTRGVYYPDKVFRSSVLHSTQDLFLPFFSNVTWFHAISGTNGTKRFDNPVLPFNDGVYFASTEKSNI
IRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDG
YFKIYSKHTPINLVRDLPQGFSALEPLVDLPIGINITRFQTLLALHRSYLTPGDSSSGWTAGAAAYYVGYLQPRTFLLKYNENGTITDAVDCALDPLS
ETKCTLKSFTVEKGIYQTSNERVQPTESIVRFPNITNLCPFGEVFNATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGNYNYLYRLFRKSNLKPFERDISTEMYQAGSTPCNGVKGFNCYFPLQ
SYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCS
FGGVSVITPGTNTSNQVAVLYQDVNCTEVPVAIHADQLTPTWRVYSTGSNVFQTRAGCLIGAEHVNNSYECDIPIGAGICASYQTQTNSHRRARSVAS
QSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQ
IYKTPPIKDFGGFNFSQILPDPSKPSKRSFIEDLLENKVTLADAGFIKQYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQDSLSSTASALGKLQDVVNQNAQALNTLVKQLSSNFGAISSVLNDILSR
LDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTHNTFVSGNCDVVIGIVNNTVYDPLQPELDSFKEELDKYFKNHTSPDVDLGDISGINASVVNI
QKEIDRINEVANNLNESLIDLQELGKYEQYIKWPWYIWLGFIAGLIAIVMVTIMLCCMTSCCSCLKGCCSCGSCCKFDEDDSEPVLKGVKLHYT
SEQ ID NO. 9
>vaccine09|L5R L7S S13N H69-V70-Y145-1472M E484K N501Y A570D P681H D1118H K1191N
MFVFRVSLPLVSNQCVNLTTRTQLPPAYTNSFTRGVYYPDKVFRSSVLHSTQDLFLPFFSNVTWFHAISGTNGTKRFDNPVLPFNDGVYFASTEKSNI
IRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDG
YFKIYSKHTPINLVRDLPQGFSALEPLVDLPIGINITRFQTLLALHRSYLTPGDSSSGWTAGAAAYYVGYLQPRTFLLKYNENGTITDAVDCALDPLS
ETKCTLKSFTVEKGIYQTSNERVQPTESIVRFPNITNLCPFGEVFNATRFASVYAWNRKRISNCVADYSVLYNSASFSTEKCYGVSPTKLNDLCFTNV
YADSFVIRGDEVRQIAPGQTGKIADYNYKLPDDFTGCVIAWNSNNLDSKVGGNYNYLYRLFRKSNLKPFERDISTEMYQAGSTPCNGVKGFNCYFPLQ
SYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCS
FGGVSVITPGTNTSNQVAVLYQDVNCTEVPVAIHADQLTPTWRVYSTGSNVFQTRAGCLIGAEHVNNSYECDIPIGAGICASYQTQTNSHRRARSVAS
QSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKIQDSLSSTASALGKLQDVVNQNAQALNTLVKQLSSNFGAISSVLNDILSR
LDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTHNTFVSGNCDVVIGIVNNTVYDPLQPELDSFKEELDKYFKNHTSPDVDLGDISGINASVVNI
QKEIDRINEVANNLNESLIDLQELGKYEQYIKWPWYIWLGFIAGLIAIVMVTIMLCCMTSCCSCLKGCCSCGSCCKFDEDDSEPVLKGVKLHYT
SEQ ID NO. 10
>vaccine10|L5P L7S S13N P25S K417N S477N E484K S494P N501Y Y505H
MFVFPVSLPLVSNQCVNLTTRTQLSPAYTNSFTRGVYYPDKVFRSSVLHSTQDLFLPFFSNVTWFHAIHVSGINGTKRFDNPVLPFNDGVYFASTEKS
NIIRGWIFGTTLDSKTQSLLIVNNATNVVIKVCEFQFCNDPFLGVYYHKNNKSWMESEFRVYSSANNCTFEYVSQPFLMDLEGKQGNEKNLREFVFKN
IDGYFKIYSKHTPINLVRDLPQGESALEPLVDLPIGINITRFQTLLALHRSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFNATRFASVYAWNRKRISNCVADYSVLYNSASFSTFKCYGVSPTKLNDLCF
TNVYADSFVIRGDEVRQIAPGQTGNIADYNYKLPDDFTGCVIAWNSNNLDSKVGGNYNYLYRLERKSNLKPFERDISTEIYQAGNTPCNGVKGFNCYF
PLQPYGFQPTYGVGHQPYRVVVLSFELLHAPATVCGPKKSTNLVKNKCVNFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARS
VASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAIGKIQDSLSSTASALGKLQDVVNQNAQALNTLVKQLSSNFGAISSVLNDI
LSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

### Example 3—Vaccine Design

Example 3 discusses vaccine candidates identified using a method embodying the concepts discussed above. A 2020-version A1 model was trained using only sequences available before Jan. 1, 2021 in this example. The A1 model was trained using 9,000 unique gene sequences encoding SARS-CoV-2 spike proteins that were first sequenced in 2020, observed at least three times, and are at least five mutations away from each other.

A selection process was performed using an evolutionary method of the type described above. In particular, the method maximized the log-likelihood and epitope scores using a MAP-Elite grid and pareto fronts as described in steps 705 to 707 above. The method mutated retained variants in nucleotide space and repeated allowing for up to 20 mutations per sequence. The method in this example minimized the semantic changes with respect to two distinct SARS-CoV-2 variants: Beta (B.1.351) and Delta (B.1.617.2).

The aim of this method in this example is to produce a realistic spike protein sequence, which is predicted to be as “correct” as the naturally occurring sequences (i.e. has comparable or better log-likelihood) but is more effective at evading immune response. By providing an immunogen which escapes the first-line immune response, the immune system can be trained to recognize other aspects of the immunogen, as well as recognize the hallmark escape mutations as immunogenic (contrary to the vaccine based on wild type sequence).

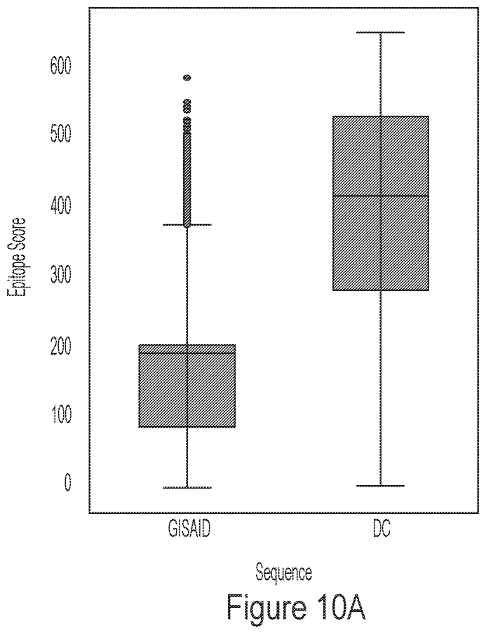

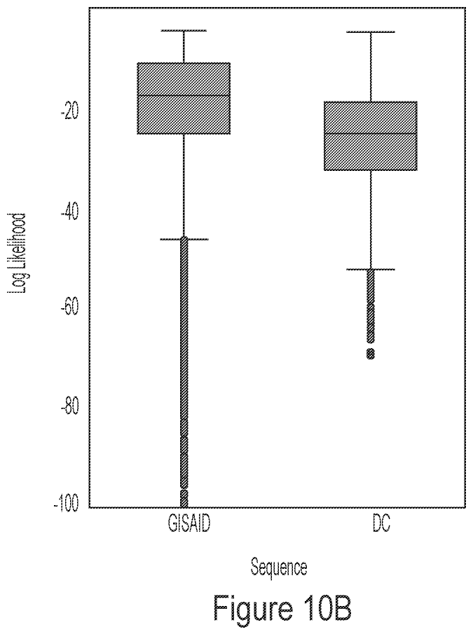

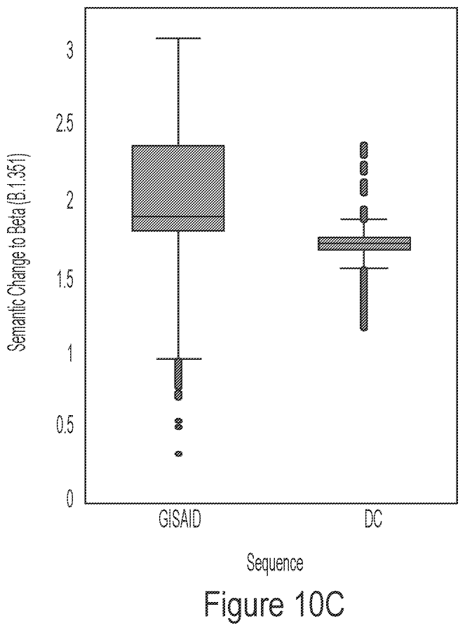

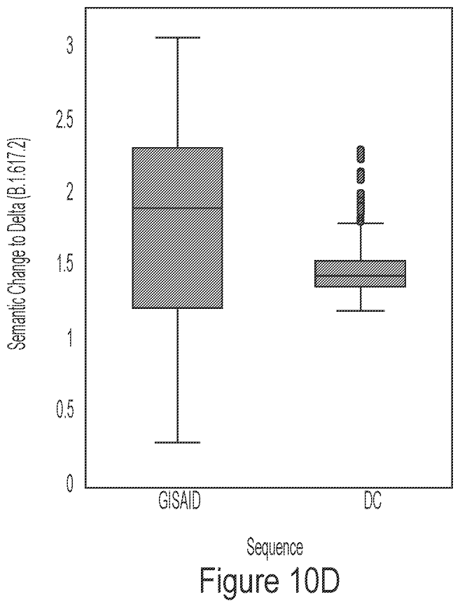

FIG. 10A shows the epitope scores for sequences from the GISAID data set and the epitope scores of sequences generated in this example, which are labelled ‘DC’. A comparison of the sequences obtained from the optimization method in this example with the sequences found in the GISAID dataset showed that sequences generated in this method have higher epitope scores, which means they could potentially evade more antibodies. It is further expected that the sequences identified by the example method will elicit more diverse antibodies.

FIG. 10B shows log likelihoods for sequences from the GISAID data set and log likelihoods of the sequences generated using the method in this example, which are again labelled ‘DC’. The sequences identified by the example method have reasonable log-likelihood values which, while not higher than the naturally occurring sequences, suggest that they are likely to be viable and would be recognized as adequate examples of an archetypal coronavirus.

FIGS. 10C and 10D show distances in embedding space (labelled “semantic change”) of sequences in the GISAID data set and the sequences generated by the method in this example from the Beta variant and the Delta variant respectively. As can be seen from these figures, the sequences identified by the example method have smaller differences compared to Beta and Delta, which means they are more likely to induce a favourable response to Beta and Delta variants.

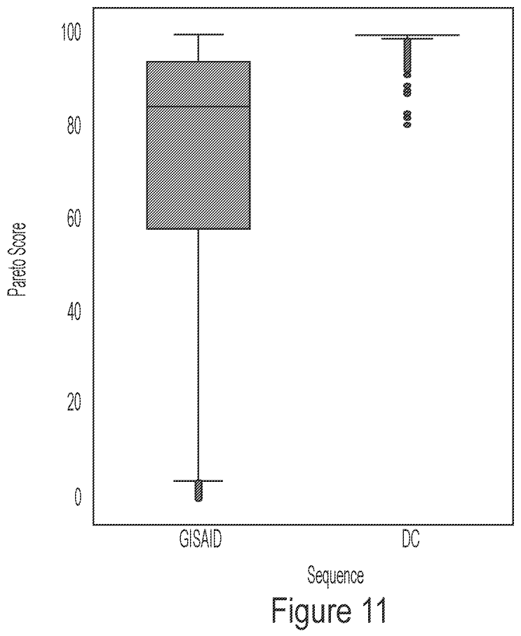

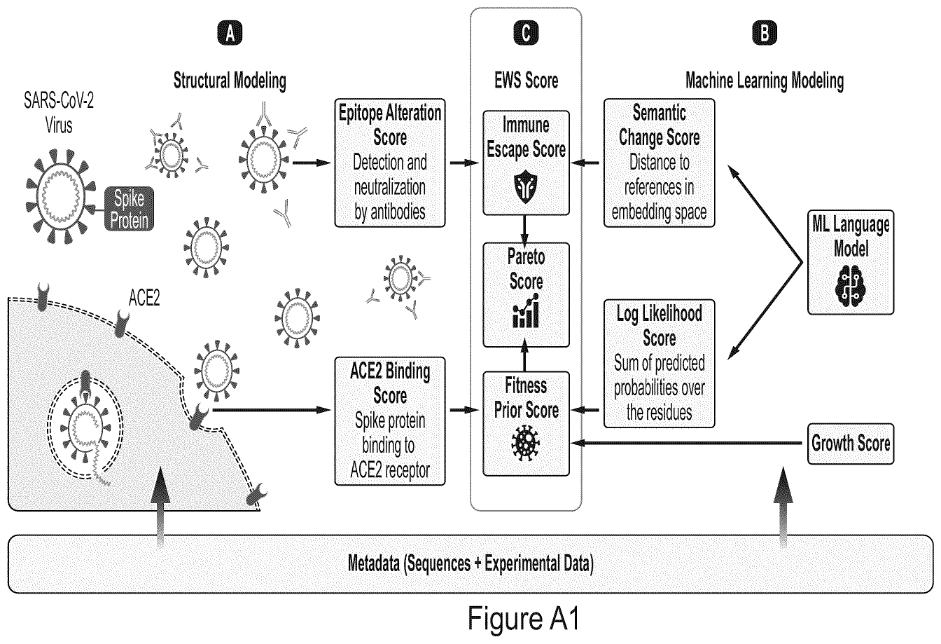

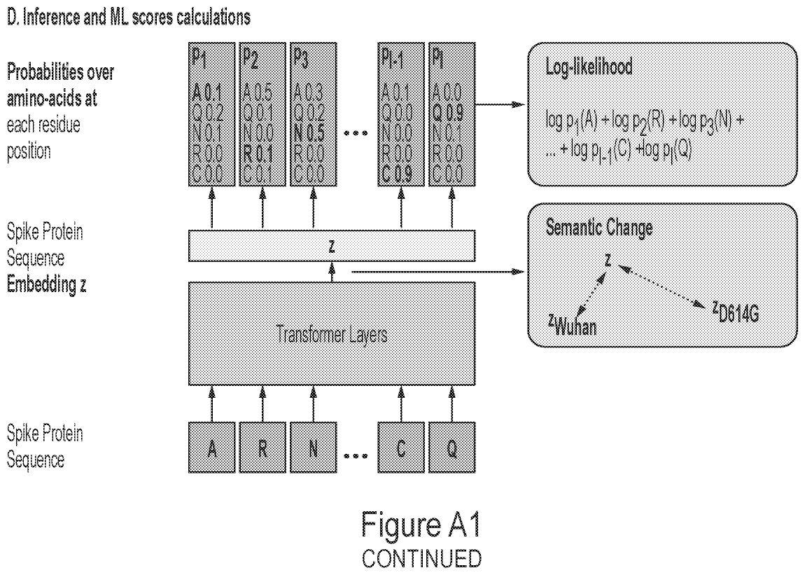

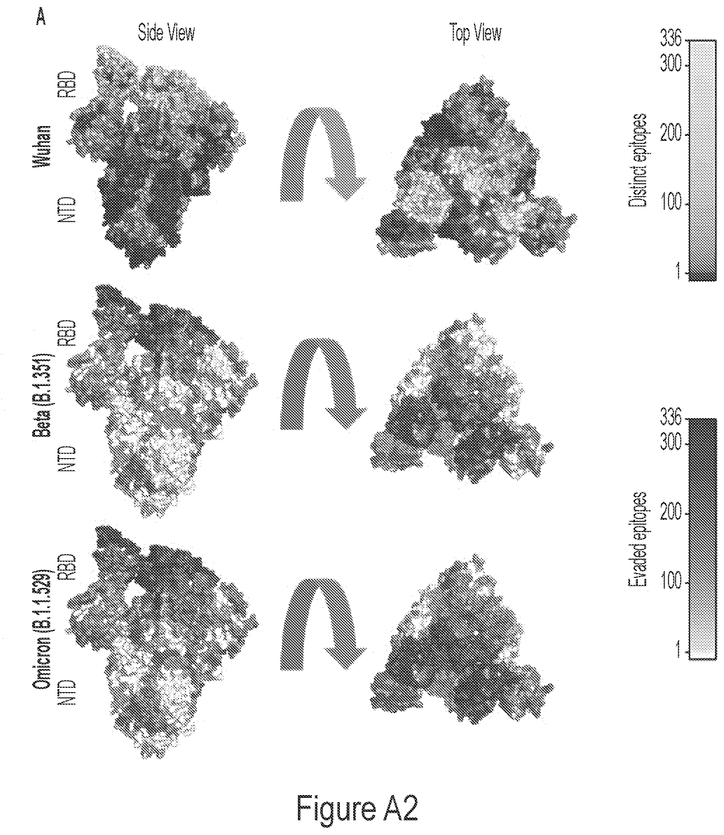

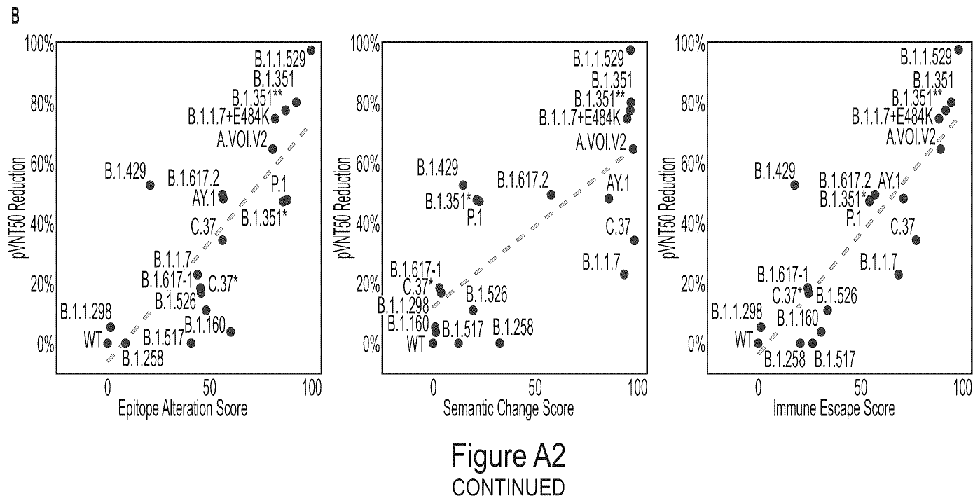

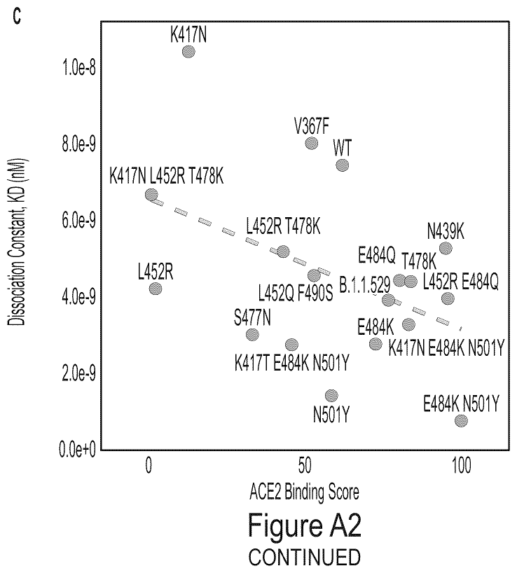

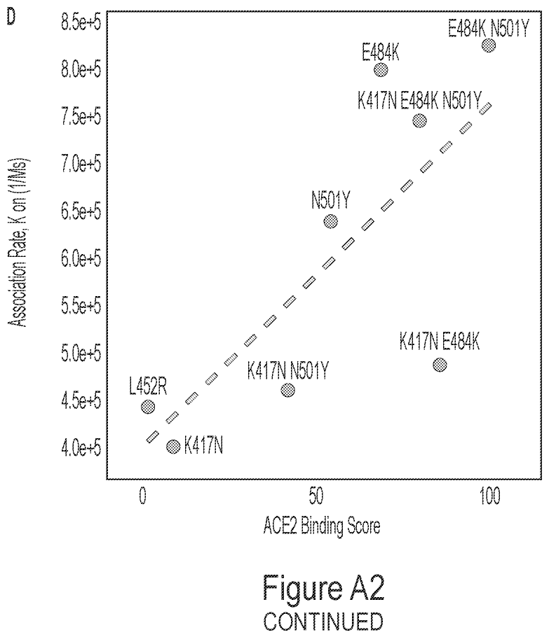

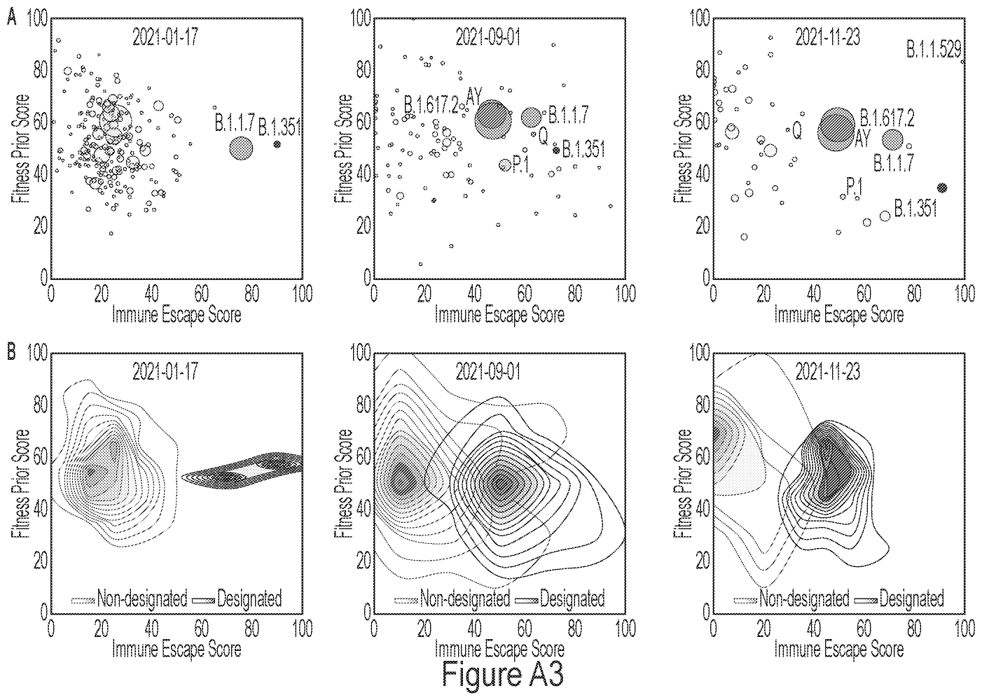

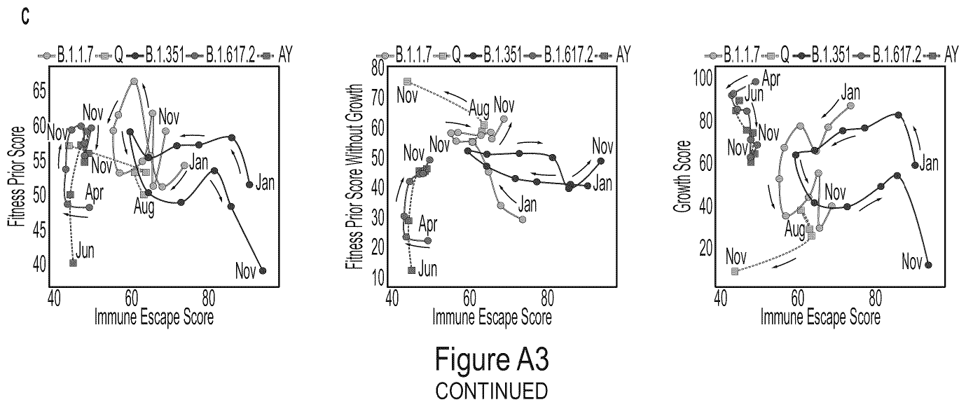

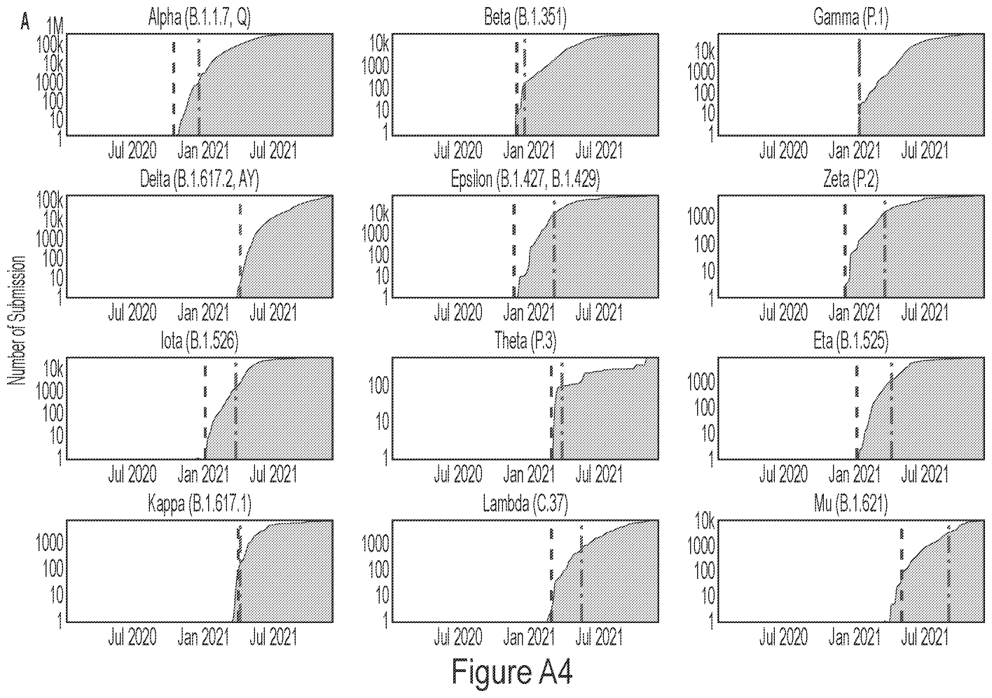

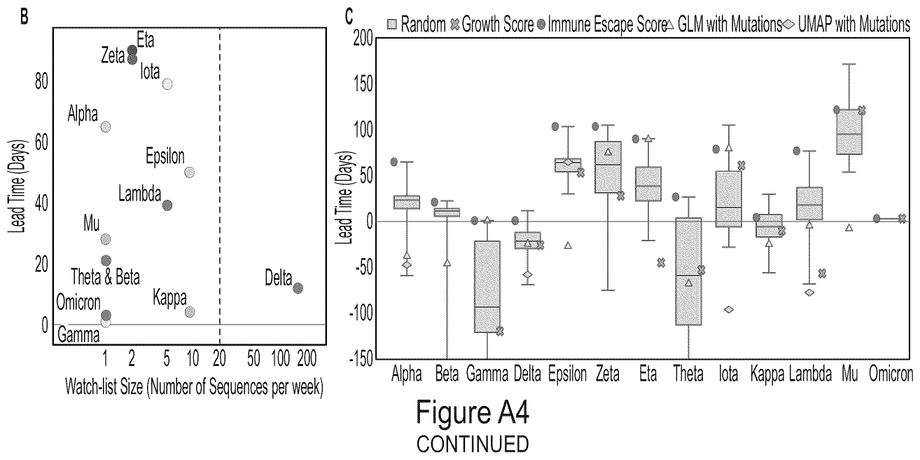

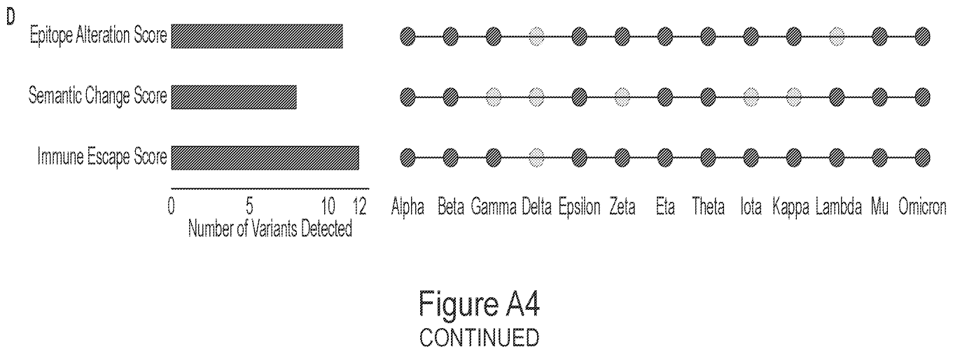

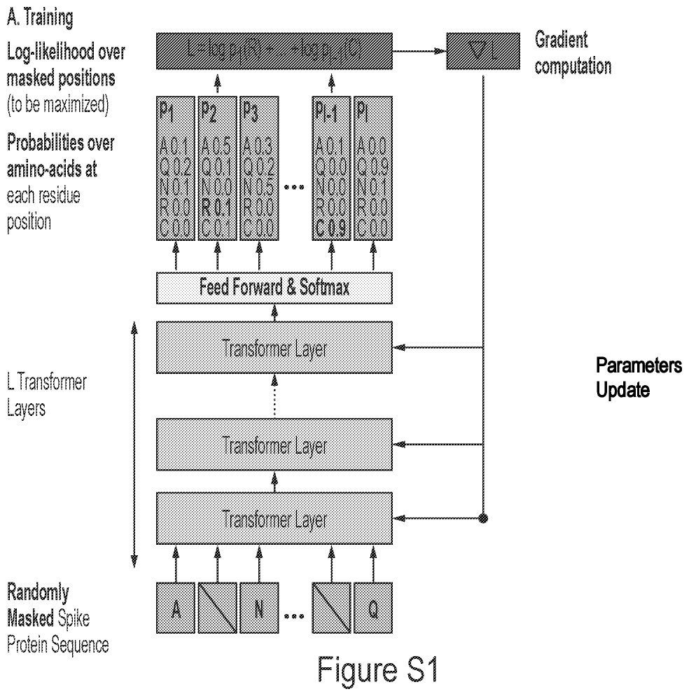

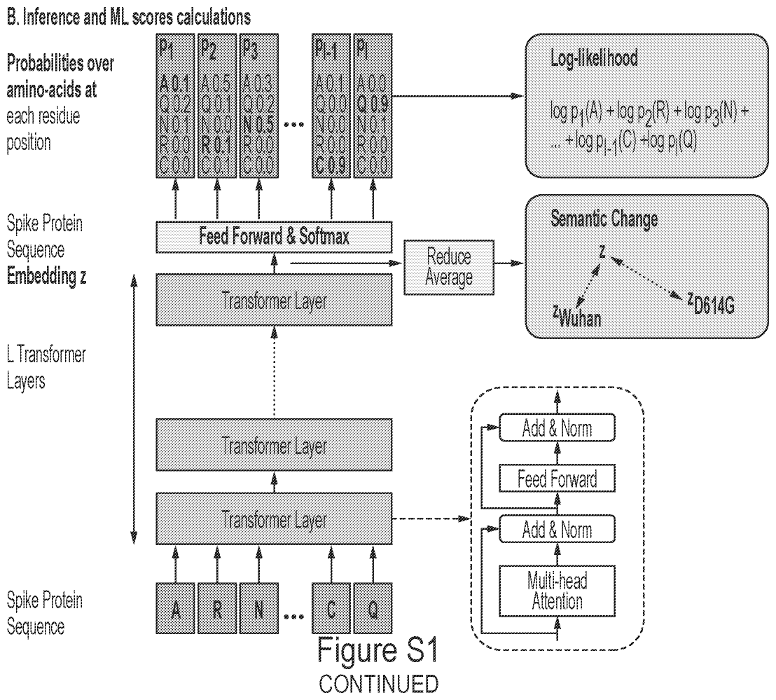

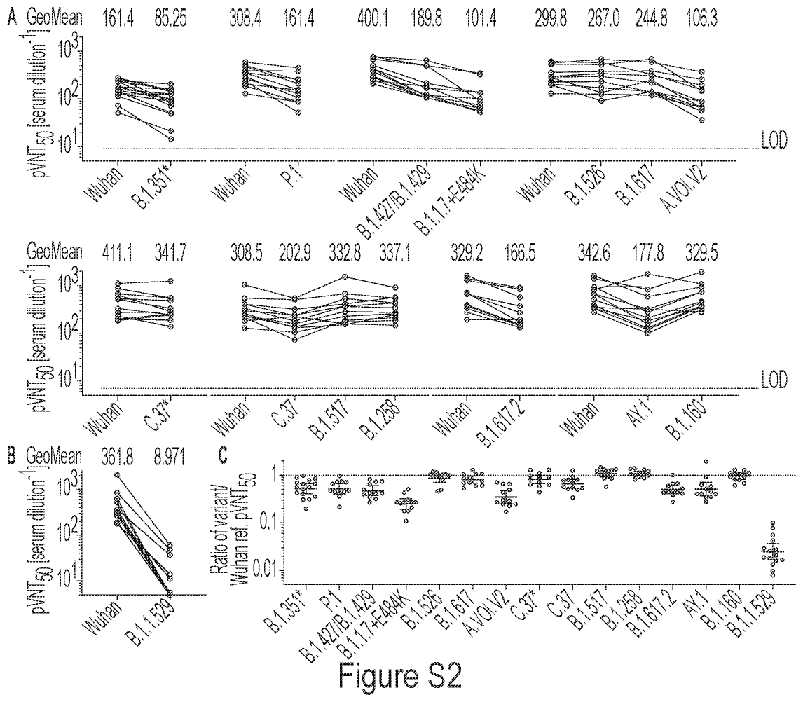

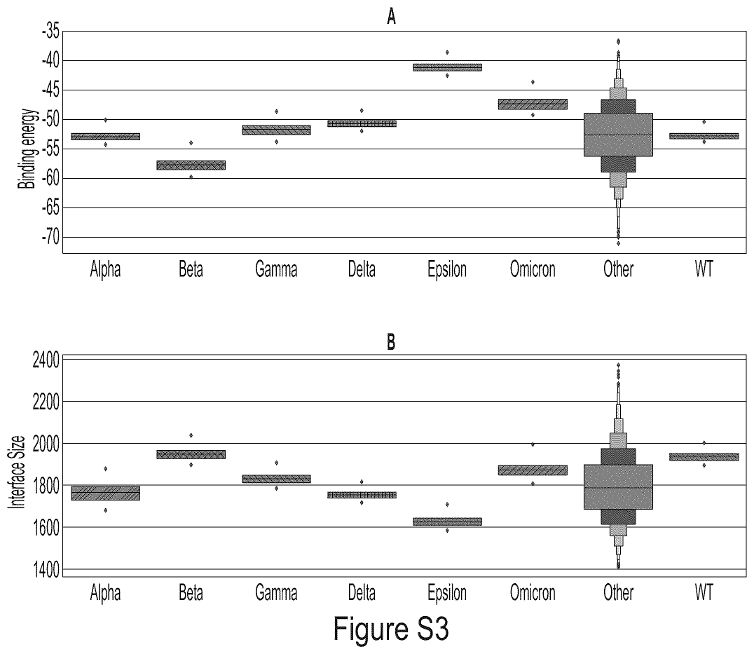

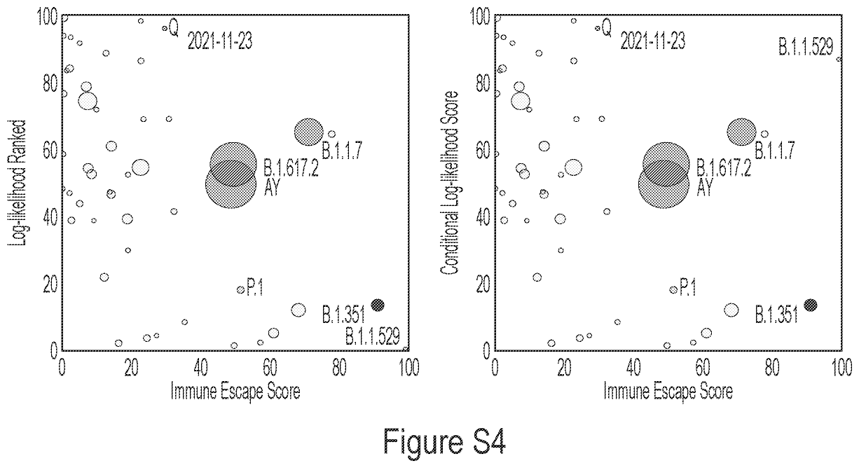

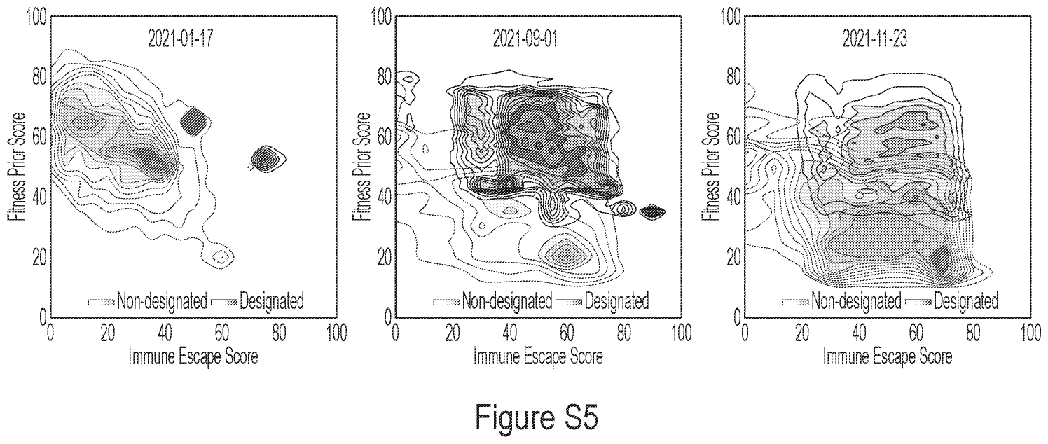

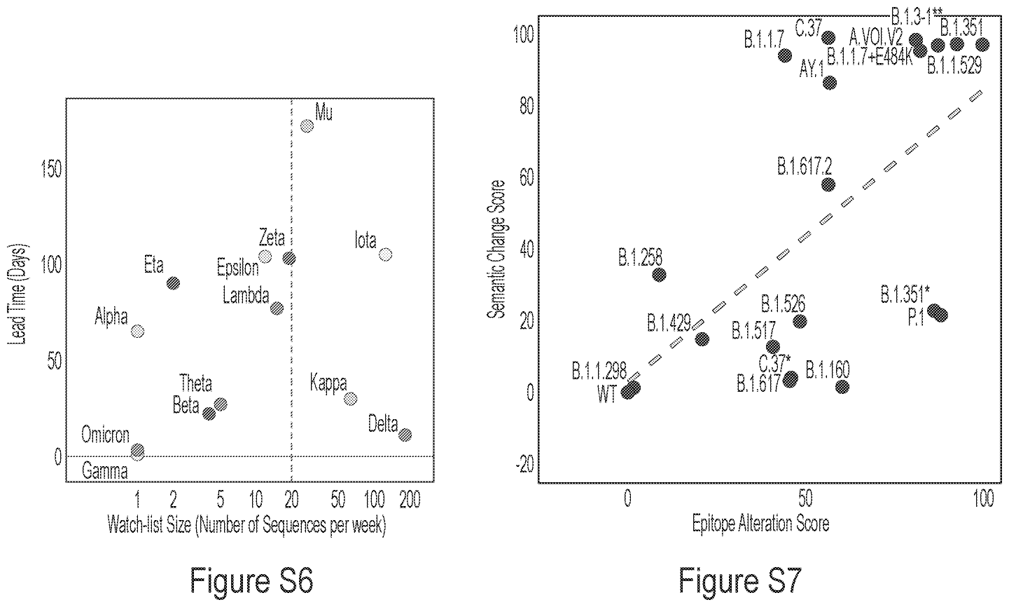

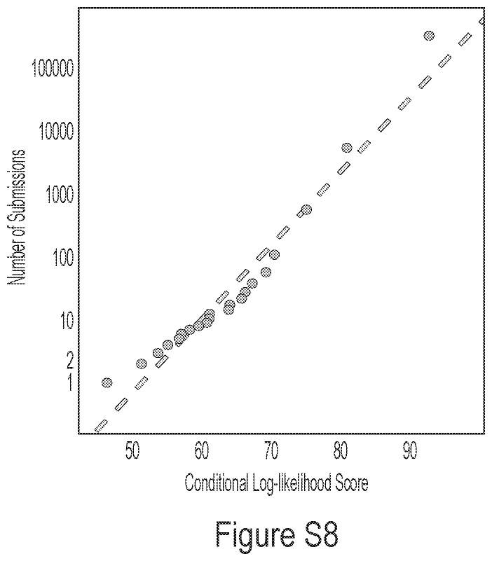

To further compare the sequences generated by the example method, a Pareto score including a factor ranking a smaller distance to the Beta and Delta sequences was calculated. The Pareto scores for the GISAID data set and the sequences from this example are illustrated in FIG. 11. A higher Pareto score indicates there are fewer sequences that have a higher epitope score, log-likelihood, and a smaller semantic change to Beta and Delta sequences. It is expected that sequences with a higher pareto score will be more suitable to be a vaccine candidate against Beta and Delta coronavirus.

As can be seen from FIG. 11, most sequences generated by the method in this example have a Pareto score close to 100, meaning that most of the sequences found are better vaccine candidates than the naturally existing sequences. This result suggests that nearly all the proposed vaccine sequences are simultaneously more grammatical (have better log-likelihood), closer to Beta and Delta variants, and likely to escape more antibodies, than any of the 4.5 million protein sequences observed in nature. The inventors consider it likely that a construct based on such sequences will be a better vaccine candidate than anything sampled from the nature.

For a polyvalent vaccine, it is possible to select a number of sequence designs, which while maximizing the Pareto score would be most distinct from each other. This would allow the polyvalent vaccine to induce an even wider range of immune responses.

### Example 4: Early Computational Detection of Potential High Risk SARS-CoV-2 Variants

### SUMMARY

The ongoing COVID-19 pandemic is leading to the discovery of hundreds of novel SARS-CoV-2 variants on a near daily basis. While most variants do not impact the course of the pandemic, some variants pose significantly increased risk when the acquired mutations allow better evasion of antibody neutralization in previously infected or vaccinated subjects, or increased transmissibility. Early detection of such high-risk variants (HRVs) is paramount for the proper management of the pandemic. However, experimental assays to determine immune evasion and transmissibility characteristics of new variants are resource-intensive and time-consuming, potentially leading to delayed appropriate responses by decision makers. The present example provides results of an in silico approach combining spike protein structure modelling and large protein transformer language models on spike protein sequences to accurately rank SARS-CoV-2 variants for transmissibility factors and immune escape potential. Among other things, the present example documents that transmissibility and immune escape metrics can be combined for an automated Early Warning System (EWS) that is capable of evaluating new variants in minutes and risk monitoring variant lineages in near real-time. In early detection mode, the EWS flagged 12 out of 13 variants, designated by the World Health Organization (WHO, Alpha-Omicron) as potentially dangerous, weeks and sometimes months ahead of them being designated as such, demonstrating its ability to help increase preparedness against future variants. Omicron was flagged by EWS on the day its sequence was made available, with immune evasion and binding metrics subsequently confirmed through in vitro experiments.

### Introduction

Despite a relatively slow mutation rate in the human coronaviruses, since the emergence of the human coronavirus SARS-CoV-2 in Wuhan in December 2019, over 250,000 different missense variants (as of Nov. 25, 2021) have been identified in the protein-coding viral sequences deposited in the GISAID (Global Initiative on Sharing All Influenza Data) database and associated with multiple lineages. Of these, over 12,750 individual missense mutations (including indels) have been observed in the spike protein, the key target for antibody neutralization (While most mutations either reduce the overall fitness of the virus, or bear no consequences to its features, some individual or combinations of mutations lead to high risk variants (HRVs), with modified immune evasion capabilities, and/or improved transmissibility. For example, the Alpha (B.1.1.7) variant of concern (VOC) spread widely through higher transmissibility compared with the Wuhan strain, while the Beta (B.1.351) VOC has been shown to be less effectively neutralized by both convalescent sera and antibodies elicited by approved COVID-19 vaccines (Liu et al., 2021b). The Delta (B.1.617.2) variant characterized by a high transmissibility led to increased mortality and triggered a renewed growth in cases in countries with both high and low vaccination rates (such as the United Kingdom (Twohig et al., 2021) and India (Singh et al., 2021)). Most recently, the more heavily mutated Omicron (B.1.1.529) variant was amongst the quickest variants to be designated as a VOC by the WHO, due to a combination of widespread dissemination and several concerning mutations in the Spike protein as well as in other proteins (The Technical Advisory Group on SARS-CoV-2 Virus Evolution (TAG-VE), 2021).

Hundreds of new variants are sequenced daily, some of which are added to the GISAID and other databases (Hatcher et al., 2017; Shu and McCauley, 2017). As new sequences continue to naturally emerge, the potential for the generation of variants that are both fit (including, e.g. highly transmissible) and highly immune resistant creates a significant challenge for public health authorities. The transmissibility and immune escape potential of a given variant could be assessed experimentally: evaluating one aspect of the fitness (e.g. transmissibility) of variants requires experimental measurements of their binding affinity with its human receptor, angiotensin-converting enzyme 2 (ACE2), which is necessary for host cell infection; assessing immune escape potential requires in vitro neutralization tests involving serum from vaccinated subjects or serum from patients previously infected with other variants of SARS-CoV-2. Both methods are resource-intensive, time-consuming, and cannot be scaled to properly address the multitude of emergent variants.

The present disclosure describes and/or utilizes technology to evaluate SARS-CoV-2 variants based on in silico structural modelling and artificial intelligence (A1) language modelling, which technology captures features of a given variant's transmissibility as well as its immune escape properties (Fig. A1). This approach is used here to build an Early Warning System (EWS) that trains on the complete (up to a chosen time point) GISAID variants database in less than a day and can score novel variants within minutes. It is a non-trivial task, as newly emerging High Risk Variants most often comprise new sets of mutations, and not all combinations of mutations present in previously identified concerning variants actually lead to enhanced immune evasion or transmissibility. In particular, accomplishing this by standard ML methods results in poorer predictive performance (see below, “Detecting HRVs by standard ML methods”). The EWS is fully scalable as new variant data become available, allowing for the continuous risk monitoring of variant lineages and has flagged HRVs weeks and sometimes months earlier than their designation as such by the WHO, providing an opportunity to shorten the response time of health authorities.

### Results:

### In Silico Prediction of Immune Escape Potential

Mutations in the spike (S) protein, especially the receptor-binding domain (RBD), may play a role in the heightened resistance to antibody-mediated neutralization of new SARS-CoV-2 variants (Weisblum et al., 2020). To evaluate the impact of said mutations on humoral immune evasion, the 336 binding epitopes observed in 310 previously resolved structures of neutralizing antibodies (nAbs) (Barnes et al., 2020; Dejnirattisai et al., 2021; Ju et al., 2020; Yan et al., 2021) were mapped onto the S protein based on publicly available resolved 3D structures (Table S.1). An overlay of all nAb:S protein interaction interfaces was used to generate a color-coded heatmap, indicating which surface-exposed amino acids are located in high epitope density regions (Fig. A2.A). The number of known nAbs whose binding epitope is affected by a distinct SARS-CoV-2 variants' mutations was defined as the epitope score (Table S.2).

While this score can be used as a first proxy to evaluate escape from humoral immunity, it may be limited by its dependence on the quantity of available antibody structure data. Herein, deep learning language models, were used to leverage information from hundreds of thousands of SARS-CoV-2 S protein sequences deposited to the GISAID database. It was recently demonstrated that these algorithms have the ability to capture biological properties of proteins through the unsupervised learning on large amounts of biological data (Elnaggar et al., 2020; Rives et al., 2021; Steinegger et al., 2019). At time of inference, a language model returns the predicted probability distribution of the twenty natural amino acids for each position in the protein, thus leveraging underlying biology of the large amount of sequences seen during training from an evolutionary point of view. Hie et al. (Hie et al., 2021) showed that language models trained on a dataset of proteins can be used to assess the risk of a viral variant. This risk was measured through two proxies named grammaticality as a measure for fitness and semantic change to assess antigenic variation. In the approach described herein, the recurrent neural networks used in (Hie et al., 2021) were replaced with attention-based models, namely transformers (Vaswani et al., 2017), hence replacing the auto-regressive way of training the model used in (Hie et al., 2021) by the BERT (Bidirectional Encoder Representations from Transformers) protocol. Even though the GISAID dataset contains hundreds of thousands of spike protein sequences, it is limited to SARS-CoV-2. To learn more general features of protein sequences and address currently unseen viral variants, one would need to use more comprehensive protein sequence resources, such as the UniProtKB database that includes hundreds of millions of protein sequences (UniProt Consortium, 2019). To benefit from this large volume of available data, the model was first pre-trained over the large collection of varied proteins included in UniProt50 and/or UniRef100 (non-redundant sequence clusters of UniProtKB and selected UniParc records) and then fine-tuned over S protein sequences. The transformer model has been re-trained every month on the variants registered in GISAID (up to 250,000 unique S sequences vs. 4,172 S sequences in Hie et al. (Hie et al., 2021)). The semantic change calculation was extended by computing it to estimate the change with respect to the wild type and from the D614G mutation to take into account this mutant that largely replaced the Wuhan strain (Fig. A2.A). The same transformer model was leveraged to calculate the log-likelihood of the input sequence: the likelihood of occurrence of a given input sequence. The higher the log-likelihood of a variant, the more probable is the variant to occur from a language model perspective. In particular, the log-likelihood metric supports substitutions, insertions and deletions without requiring a reference.

In vitro pseudovirus neutralization test (pVNT) assays were used to validate the immune escape in silico metrics: semantic change and epitope score. The cross-neutralizing effect of n>12 BNT162b2-immune sera was assessed against vesicular stomatitis virus (VSV)-SARS-CoV-2-S pseudoviruses bearing the spike protein of n=19 selected High Risk Variants, including Omicron (B.1.1.529) (Fig. A2.B, Fig. S2, Table S.3) (Muik et al., 2021; Sahin et al., 2021). The SARS-CoV-2 Omicron pseudovirus was by far the most immune escaping with >20-fold reduction of the 50% pseudovirus neutralisation titer (pVNT₅₀) compared with the geometric mean titer (GMT) against the Wuhan reference spike-pseudotyped VSV (Fig. S2.B). The calculated geometric mean ratio with 95% confidence interval (CI) of the Omicron pseudotype and the Wuhan pseudotype GMTs was 0.025 (95% CI; 0.017 to 0.037), indicating another 10-fold drop of the neutralising activity against Omicron compared to the second most immune escaping B.1.1.7+E484K pseudovirus with a geometric mean ratio of 0.253 (95% CI; 0.196 to 0.328) (Fig. S2.C). This result is correlates with the in silico immune escape score for Omicron which is the highest amongst observed, circulating variants. Across all HRV pseudoviruses tested, both the epitope score and the semantic change score correlate positively with the calculated pVNT₅₀ reduction (Fig. A2.B; Pearson r=0.79 and 0.74, respectively). Of note, an average of both in silico scores (summarized as the ‘immune escape score’) exhibits a stronger correlation with the observed reduction in neutralizing titers (Pearson r=0.85).

In silico estimation of infectivity or fitness.

The immune escape score predicts if a given viral variant may evade neutralization by the immune system, but it does not capture protein changes that either enhance efficacy of viral cell entry, or negatively impact its structure or function. Capturing the full transmissibility potential of the virus (fitness also referred to herein as infectivity) may involve many complex dynamics. Described herein are at least three informative factors contributing toward it: ACE2 binding score, conditional log-likelihood score and growth.

One determinant of viral spread is the effectiveness with which virus particles can attach to and invade target host cells. This characteristic may be especially important when considering individuals without pre-existing immunity or viral variants which are able to better evade immune responses. To infect the human host cell, the RBD of the viral S protein associates with ACE2, the cellular receptor for SARS-CoV-2. Infectivity was assessed based on the predicted impact of sets of mutations on the binding affinity of the variant S protein to the human ACE2 receptor, here referred to as the ACE2 binding score. The interaction between a variant S protein and the ACE2 protein was computed through repeated, fully flexible, in silico docking experiments, allowing for unbiased sampling of the binding landscape. In order to reduce the required computational resources, spike protein modelling was restricted to its RBD domain, i.e. the domain known to directly bind to the ACE2 receptor. To calculate the ACE2 binding score, the median binding energy (that is the difference estimated Gibbs free energy 6G between bound and unbound states) is used, which acts as a proxy for global complex affinity (Fig. S3).

In order to assess the validity of the ACE2 binding score, the simulation results were compared with in vitro results. Surface plasmon resonance (SPR) kinetic analysis was performed to determine the affinity (KD, dissociation constant) of 19 RBD variants to the ACE-2 receptor. Notably, the assay measures observable association rates, which are a result of a dynamic process, while simulations measure aggregated, static binding affinity, thus marginalising the contribution of mutations toward the flexibility and kinetics of the spike protein. Despite this, the ACE2 binding score used herein shows meaningful correlation with the KD values with a Pearson correlation coefficient of 0.45. (Fig. A2.C).

In a further example, in order to assess the validity of the ACE2 binding score, the simulation results were compared with in vitro results reported by Tanaka et al. (Tanaka et al., 2021). The authors of that paper performed a biolayer interferometry (BLI) kinetic analysis to measure the association rate (kon) for targeted sets of mutations and showed the relationship between RBD mutations and the RBD-ACE2 association rate. The ACE2 binding score shows meaningful correlation with the association rate (kₒₙ) for targeted sets with a Pearson correlation coefficient of 0.75. (Fig. A2.1).

Another aspect that partially models the fitness of a variant, is how similar a given variant is to the other variants which have been known to grow rapidly. Effective assessment of such similarity may not be achievable by simple sequence comparison, due to epistatic interactions between sites of polymorphism, in which certain mutation combinations enhance fitness while being deleterious when they occur separately. The same trained transformer model described previously was leveraged to calculate the log-likelihood of the input sequence. From a language model perspective, the higher the log-likelihood of a variant, the more probable is the variant to occur. In particular, the log-likelihood metric supports substitutions, insertions, and deletions without requiring a reference sequence to measure against, unlike the grammaticality of (Hie et al., 2021) that requires a reference sequence. The language model disclosed herein was not provided with explicit sequence count data in the training phase, yet on average assigned higher log-likelihood values to sequences with higher actual observed count. High log-likelihood may indicate features common in the general variant population, which are likely to be fitness-related, thus allowing strains harbouring these to sustain additional such mutations.

However, the values of log-likelihood tend to diminish with the increasing number of mutations, which is expected given the definition of this metric; this over-emphasizes variants with low mutation counts. Considering that all the samples used for training have been detected in patients, and as such have likely satisfied a minimal fitness criteria, the conditional log-likelihood score is introduced, measuring how the log-likelihood of the variant in question compares to other variants with similar mutational loads, as opposed to the entire population. This metric sheds more light on highly mutated, potentially concerning variants like B.1.1.529 (Omicron). Due to its high mutational load, this variant might be perceived by raw log-likelihood as highly unlikely. However, relative to other variant sequences with a similar number of mutations, it stands out, leading to a high conditional log-likelihood score (Fig. S4).

Metrics discussed above may not capture the entirety of factors affecting frequency of viral variants. Additionally, conditional log-likelihood is a metric measuring similarity to already known, rapidly increasing samples. By its nature, it cannot accurately assess variants which exhibit completely new sequence features, until these features are observed more often. The present example utilizes an infectivity metric that includes growth, an empirical term of the quantified change in the fraction of observed sequences in the database that a variant in question comprises. Variants which are increasing in prevalence may be considered to be more imminently interesting than those which are not.

### Combining Infectivity (Fitness Prior) and Immune Escape Scores to Continuously Monitor High Risk Variants

Different selective pressures on virus evolution lead to variants with high immune escape and fitness (e.g. infectivity), since a virus must remain evolutionarily competent or preserve evolutionary fitness to successfully spread. A system that keeps track of immune escape and infectivity factors can monitor (e.g. can continuously monitor) high risk variants (HRVs) on a near real-time basis, since new sequences can be evaluated and added to the data pool in minutes. The ranking of any sequence, and consequently—lineage, depends the other, circulating sequences.

As seen in Fig. A3C, variants of concern start off relatively far into the upper-right corner (i.e. are comparatively highly immune escaping and have satisfactory infectivity score for their immune escape value). Newly emerging variants diversify over time, as there are more circulating sequences observed. Their aggregated immune escape score decreases, while the infectivity score (based partially on prior observations)—increases for truly fit variants (Alpha: B.1.1.7 and Q lineages, and Delta: B.1.617.2 and AY lineages). Lineages such as Beta progressively decrease in aggregated infectivity, closely recapitulating their lack of global growth, despite continued prevalence. The effect of perceptible global growth of B.1.351 (Beta) in April 2021, as well as its drop in prevalence and acquisition of further diversifying mutations, are all visible in the plot. The case of B.1.351 (Beta) illustrates a variant that is—according to the data and statistical models—unlikely to regain global significance. Simultaneously, one needs to observe the effects of fitness-enhancing events (either mutations or emergence of evolutionary niche), such as the increase in metrics for Alpha lineages in Summer 2021, which was possibly due to the competitive effect of B.1.617.2 emergence. This evolutionary pressure could have been one of the factors behind the near eradication of B.1.1.7. The remaining sequences are under evolutionary pressure to adapt to the changing circumstances, and while currently not significant globally, still pose a tangible threat.

To jointly score the relative risks of variants using immune escape potential and fitness (e.g., infectivity), an optimality score, termed Pareto score, was used to assess variants. The Pareto score is a mathematically robust way to identify lineages that are immune escaping, infectious, and capture the relative evolutionary advantage of a given strain (see Methods section for calculation details). For each lineage, as defined by the Pango nomenclature system (Rambaut et al., 2020), scores were calculated by averaging the scores of the individual sequences belonging to a given lineage. A high Pareto score at a given time for a specific lineage indicates that only a few other lineages have higher scores for fitness and immune escape at that time.

In order to validate that the Pareto score separates WHO designated variants from non-designated variants, Welch's t-tests were conducted over the registered variants population every week from January 2021 to November 2021 (Table S.5). The null hypothesis can be rejected with a p-value <0.05, for all 50 weeks, thus demonstrating that the Pareto score can separate designated from non-designated variants continuously through time. For visualisation purposes, a focus was made on three dates of interest: the 17th of January 2021, the 1st of September 2021 and the 23rd of November 2021. At these dates, p-values=2E-143, 6E-4 and 4E-4 were reported. Density contour estimates conducted on Jan. 17, 2021, Sep. 1, 2021 and Nov. 23, 2021 also demonstrate clear separability between WHO designated variants and non-designated ones (per lineage: Fig. A3. B and per sequence: Fig. S5). Importantly, they suggest that immune escape significantly contributes to this separability, more so than the infectivity score.

### Detection of Potentially High Risk Variants Prior to Substantial Spread in Population

Experimental assays aiming to determine a given variant's immune evasion and fitness (e.g. infectivity) are time and resource-intensive. Available data show that approximately thousands of new variants are emerging every week at an increasing rate (on average ˜250 per week in September 2020, 7,000 in August 2021 and 10,000 in October 2021). Moreover, this number is likely an underestimate given limited viral sequencing and data deposition in many countries. It is therefore not feasible for health authorities to perform preventative experimental (e.g. in vitro) assessments whenever a new variant is identified, despite the benefits of a proactive stance detecting HRVs before their spread.

As seen in the previous section, the utilized EWS immune escape score helps separate WHO designated variants from non-designated variants and has demonstrated a significant correlation to in vitro neutralisation test results.

In addition, our immune escape score is computed from sequence alone and unlike the described infectivity score does not require growth metrics, which are not available when a novel variant is sequenced. This means that an early detection version of our system, operating based on immune escape score alone, could potentially spot HRVs.

Moreover, it was recently proposed that viral evolution in immunocompromised patients generates intrapatient viral variants with increased immune evasion, rather than increased fitness and constitutes a significant factor contributing to variant spread (Corey et al., 2021; Weigang et al., 2021). Some of the new variants reside on long branches of phylogenetic tree, which suggests they could have undergone an extensive intrapatient evolution enabled by the immunocompromised status of the host. These results, together with increased vaccination rates worldwide, put an added emphasis on immune evasion as a key risk factor in newly emerging variants, provided further motivation to use an approach that uses only immune escape score to detect HRVs.

A systematic analysis was conducted where for every week between September 16th, 2020 and November 23rd 2021 the EWS ranked all new sequences on immune escape to compile a weekly flagged HRV watch-list. The models were trained on variants up to the previous month's start date and any other data used was prior to the analysis date. To assess the system's sensitivity, the ability of the algorithm to detect the 13 variants designated by WHO (Alpha, Beta, Gamma, Delta, Epsilon, Zeta, Eta, Theta, Iota, Kappa, Lambda, Mu, and Omicron) was assessed.

When using a weekly watch-list with a size of 20 variants (less than 0.5% of the weekly average of new variant sequences), EWS flagged 12 WHO designated variants out of 13 (Fig. A4.A), with an average of 58 days of lead time before these were designated as such by the WHO (Table S.4). For variants Alpha to Mu for which we have sufficient data, on average only 0.5% of cases of that variant were already recorded at the time of their detection by the EWS (25 sequences on average), to be contrasted with the WHO announcements that happened on average when 18% of cases for that variant were already recorded (1,593 sequences on average). Different watch-list sizes ranging from 1 up to 200 variants were assessed to evaluate detection sensitivity (Fig. A4.B). The number of named variants detected remained stable when varying the weekly watch list size between 10 and 200. While a longer list compromises specificity, it leads to an increase in the detection lead time (the number of days ahead of WHO designation) (Fig. S6).

While the system as described in this Example did not accurately pinpoint the emergence of the B.1.617.2 Delta family of variants, the system can be modified to account for mutations that may contribute to abrogation of O-glycosylation, which further enables furin cleavage. In particular, Delta is known to be neutralised by vaccines (Liu et al., 2021a) and its global prevalence can be attributed to other fitness-enhancing factors. These factors, such as P681R mutation (Zhang et al., 2021), which abrogates O-glycosylation, thus further enabling furin cleavage. Delta variants also first emerged in India, a vast country with a diverse population and relatively limited sequencing capabilities, hence available samples may have been insufficient to fully describe the epidemiological landscape in time. Government regulations prohibiting the export of biological data out of the country may have also further restricted sequence data from reaching global databases like GISAID. Thus as more samples and biological data become available, the system can be improved.

Strikingly, WHO-designated variants Alpha, Beta, Gamma, Theta, and Omicron are detected on the same week they are first reported, even in the extreme case where the weekly watch-list allows only one variant, meaning they were the top-scoring sequences among all emerging variants that week (Fig. A4.B). In addition, using a larger weekly watch-list of 20 variants, Epsilon, Zeta, Eta and Lambda are also detected in the same week they are first reported, in addition to the previously mentioned WHO-designated variants (9 in total detected the first week).

Specifically, the EWS identified Omicron as the highest immune escaping variant over more than 70,000 variants discovered between early October and late November 2021. This variant combines frequent RBD mutations (K417N, S477N, N501Y), with less frequent ones (G339D, S371L, S373P, S375F, Q498R) to potentially evade RBD-targeting antibodies. The NTD indels in positions 69-70, 143-145, 211-214 alter known antibody recognition sites as well. These mutations, together with over 20 others, led to an exceptional epitope score, the highest recorded since the beginning of the pandemic and a high semantic change score, which combined rank Omicron in the top 0.005% of variants on immune escape since the pandemic started. Growth score alone could be considered as a plausible metric that requires neither AI nor simulation to early detect HRVs with yet better than random results. However, the immune escape score implemented in EWS outperforms the growth score across the variants where a comparison is available, in terms of lead time ahead of WHO designation. The growth score also fails to detect Delta ahead of time despite the established fitness of this variant, which may be another consequence of incomplete or delayed sequencing data (Fig. A4.C).

The immune escape score used by the EWS to early detect HRVs combines the epitope score and semantic change score (Fig. A1. A, B, C). Early detection performance of each one of these two components was evaluated separately and combined: while the Epitope Score detects 11 out of 13 WHO designated variants ahead of time, the Semantic Change score detects only 8 out of 13. Their combination, however, flags 12 out of 13 WHO designated variants (Fig. A4.D). Standard machine learning techniques, both supervised and unsupervised, were applied and the results denoted respectively “GLM with mutations” and “UMAP with mutations”, corresponding conceptually to Epitope Score and Semantic Change score (see below “Detecting HRVs by standard ML methods”). These standard machine learning techniques do not reach the same predictive performance as the methods proposed in this Example. This validates an approach of associating protein structure modelling and transformer language models on protein sequence to accurately rank SARS-CoV-2 variants.

### Discussion

Validation of the immune escape and infectivity scores using published and newly generated data, showed that the combination of structural simulations, AI, and biological nucleic sequencing of the SARS-CoV-2 variants allows for continuous risk monitoring and sensitive early detection of HRVs.

The EWS described above flags Omicron on the day it is uploaded to GISAID (Nov. 23, 2021) based on its sequence alone, as one of the highest immune escaping variants ever documented for SARS-Cov-2 sequence variants. Moreover, EWS described above assigns Omicron a high fitness prior score based on the combination of predicted ACE2 binding and a substantial conditional log likelihood score. However, the EWS described in this example has a limitation for the calculation of the conditional log-likelihood score for Omicron, which is the relatively small number of variants that have such a high number of mutations.

The Early Warning System can sensitively detect HRVs months ahead of the official WHO designation, sometimes within the same week a sequenced variant enters the database. Specifically, EWS's flagging of Delta only after its designation by the WHO, with a significant underrepresentation of the lineage in GISAID emphasises the importance of extensive, robust and timely sequencing of SARS-CoV-2 genomic samples (e.g. in a potential region and/or globally).

Combining comprehensive sequencing with structural modelling and AI can provide unprecedented insights into the COVID-19 pandemic which could be harnessed e.g. by public health authorities and governments worldwide to increase their early preparedness to HRVs and potentially alleviate the associated human and economic costs.

### Materials and Methods

The methodologies described above are described in greater detail in the following sections.

### Variant Notations

As used herein “a variant” of a spike protein refers to a protein sequence of a coronavirus' spike protein that differs from the original Wuhan spike protein (also referred to herein as the wild type spike protein). Variants are represented in terms of their mutations with respect to the Wuhan strain. For instance, the notation N501Y represents a substitution in position 501, replacing N with Y. The notation ins214AR represents inserting AR after position 213, and the notation H69-V70—represents a deletion of H and V at positions 69 and 70.

### VSV-SARS-CoV-2 S Pseudovirus Neutralization Assay

A recombinant replication-deficient VSV vector that encodes green fluorescent protein (GFP) and luciferase (Luc) instead of the VSV-glycoprotein (VSV-G) was pseudotyped with SARS-CoV-2 spike (S) protein derived from either the Wuhan reference strain (NCBI Ref. 43740568) or variants of interest according to published pseudotyping protocols (Berger Rentsch and Zimmer, 2011). The mutations found in S of the VOCs are listed in Table S.3. In brief, HEK293T/17 monolayers transfected to express SARS-CoV-2 S with the C-terminal cytoplasmic 19 amino acids truncated (SARS-CoV-2-S[CΔ19]) were inoculated with the VSVΔG-GFP/Luc vector. After incubation for 1 hour at 37° C., the inoculum was removed, and cells were washed with PBS before medium supplemented with anti-VSV-G antibody (clone 8G5F11, Kerafast) was added to neutralize residual input virus. VSV-SARS-CoV-2 pseudovirus-containing medium was collected 20 hours after inoculation, 0.2 μm filtered and stored at −80° C.

For pseudovirus neutralization assays, 40,000 Vero 76 cells were seeded per 96-well. Sera were serially diluted 1:2 in culture medium starting with a 1:15 dilution (dilution range of 1:15 to 1:7,680). VSV-SARS-CoV-2-S pseudoparticles were diluted in culture medium to obtain either ˜1,000 or ˜200 transducing units (TU) in the assay. Same input virus amounts for all pseudoviruses were used within an individual experiment. In total 8 experiments were performed covering the SARS-CoV-2 variants listed in Table S.3 always taking Wuhan S pseudovirus as reference. Serum dilutions were mixed 1:1 with pseudovirus for 30 minutes at room temperature prior to addition to Vero 76 cell monolayers and incubation at 37° C. for 24 hours. Supernatants were removed, and the cells were lysed with luciferase reagent (Promega). Luminescence was recorded, and neutralization titres were calculated by generating a four-parameter logistic fit of the percent neutralization at each serial serum dilution. The pVNT₅₀ is reported as the interpolated reciprocal of the dilution yielding a 50% reduction in luminescence. If no neutralization yielding a 50% reduction in luminescence was observed, an arbitrary titer value of 7.5, half of the limit of detection (LO), was reported.

### Binding Kinetics of RBD Variants to ACE2 Using Surface Plasmon Resonance Spectroscopy

Binding kinetics of RBD variants may be determined using a Biacore T200 device (Cytiva) with HBS-EP+ running buffer (BR100669, Cytiva) at 25° C. Carboxyl groups on the CM5 sensor chip matrix were activated with a mixture of 1-ethyl-3-(3-dimethylaminopropyl) carbodiimide hydrochloride (EDC) and N-hydroxysuccinimide (NHS) to form active esters for the reaction with amine groups. anti-mouse-Fc-antibody (BR100838, Cytiva) was diluted in 10 mM sodium acetate buffer pH 5 (30 μg/mL) for covalent coupling to immobilisation level of ˜6,000 response units (RU). Free N-hydroxysuccinimide esters on the sensor surface were deactivated with ethanolamine-HCl.

Human ACE2-mFc (10108-H05H, Sino Biological Inc.) was diluted to 5 μg/mL with HBS-EP+ buffer and applied at 10 μL/min for 15 seconds to the active flow cell for capture by immobilised antibody, while the reference flow cell was treated with buffer. Binding analysis of captured hACE2-mFc to RBD variants (40592-V08B, 40592-V08H113, 40592-V08H115, 40592-V08H82, 40592-V08H59, 40592-V08H84, 40592-V08H85, 40592-V08H112, 40592-V08H28, 40592-V08H81, 40592-V08H90, 40592-V08H91, 40592-V08H88, 40592-V08H86, 40592-V08H111, 40592-V08H80, 40592-V08H1, 40592-V08H14, 40592-V08H46, 40592-V08H121 40592-V08H121, Sinobiological Inc.) was performed using a multi-cycle kinetic method with concentrations ranging from 3.125 to 50 nM. An association period of 120 seconds was followed by a dissociation period of 300 seconds with a constant flow rate of 30 μL/min and a final regeneration step. Binding kinetics were calculated using a global kinetic fit model (1:1 Langmuir, Biacore T200 Evaluation Software Version 3.1, Cytiva).

### Data

The genomic sequences and Spike protein sequences were collected from GISAID. For each Spike protein sequence, the missing amino acids were filled using the next known amino acid and the lineage assignment was performed using PANGOLIN (O'Toole et al., 2021). Mutations with respect to the wild type were calculated using Clustal Omega (3. Sievers, F. et al., 2019) and HH-suite (Steinegger et al., 2019).

The GISAID dataset is imbalanced towards some lineages that have been more prevalent and because certain regions have performed more sequencing than others. To mitigate this bias in the dataset during training, the importance of each sequence is weighted differently in the loss calculation. The importance of a sequence is defined as

$${w}_{s} ∝ {\sum }_{( s , l ) \sim D} {log}_{10} ( {c}_{s} ) + {log}_{10} ( {c}_{( s , i )} ) + {log}_{10} ( {c}_{l \vert s} )$$

where the values cₛ and c_{s,l} are the numbers of occurrences in the dataset of the sequence s and the sequence-laboratory pair (s, l), respectively. The value c_{l|s} corresponds to the number of laboratories having reported sequence s, which measure the prevalence across regions of the variant.

The model excludes from training all the sequences which have been observed only once in the data set. In this way it eliminates spurious changes, due to sequencing errors, as well as samples of viruses of subpar evolutionary fitness, which do not spread between patients.

### Language Modelling

The domain of Natural Language Processing (NLP) has experienced several breakthroughs in the past years. The emergence of recurrent and attention-based deep neural networks led to impressive results for text generation and translation. Recently, this technology has been leveraged to learn the language of biology (Elnaggar et al., 2020; Rives et al., 2021). It works with a simple analogy where protein sequences are considered as sentences and the amino acids as words. The models are trained on large datasets of known protein sequences in an unsupervised manner. In other words, there is no need to label the data and any newly registered protein sequence can be exploited.

Information about protein properties is stored at two positions inside the model once it is trained. On one side, the probabilities returned by the model indicate how likely this sequence is to be natural/viable/feasible. On the other hand, the outputs of the model's layers and notably the last layer provide a high dimensional representation for each sequence, referred to herein as embedding of the protein. The embedding of the protein contains information about the protein properties and can be used either directly or to train a classification or regression model. Recently, (Meier et al.) demonstrated that these models also capture the effects of mutations on protein function (Meier et al., 2021).

### Model Architecture

In the methods utilized herein, the input of the model consists of the sequence characters corresponding to the amino acids forming the protein. Each amino acid is first tokenized, i.e. mapped to their index in the vocabulary containing the 20 natural amino acids and then projected to an embedding space. The sequence of embeddings is then fed to the transformer model (Vaswani et al., 2017) consisting of a series of blocks, each composed of a self-attention operation followed by a position-wise multi-layer network (Fig. S1).

Self-attention modules explicitly construct pairwise interactions between all positions in the sequence which enable them to build complex representations that incorporate context from across the sequence. Because the self-attention operation is permutation-equivariant, a positional encoding must be added to the embedding of each token to distinguish its position in the sequence.

### Self-Supervised Training

Given a large database of protein sequences, the model can be trained using the masked language modeling objective presented in [31]. Each input sequence is corrupted by replacing a fraction of the amino acids with a special mask token. The network is then trained to predict the missing tokens from the corrupted sequence. In practice, for each sequence x, we randomly sample a set of indices i∈M, for which the amino acid tokens are replaced by a mask token, resulting in a corrupted sequence \hat{x}. During pre-training, the set M is defined such that 15% of the amino acids in the sequence get corrupted. When corrupted an amino acid has 10% to be replaced by another randomly selected amino acid and 80% being masked. During fine-tuning these probabilities do not change, however, only 2.5% of the amino acids in the sequence get corrupted. This probability was selected in order to enable the model to become more accurate for spike protein sequences while keeping its performance on diverse sequences from UniRef100. The training objective corresponds to the negative log-likelihood of the true sequence at the corrupted positions.

$${L}_{θ} ( \hat{x} \vert x ) = - {\sum }_{i ∈ M} log p ( {x}_{i} \vert \hat{x} )$$

To minimize this loss, the model must learn to identify dependencies between the corrupted and uncorrupted elements of the sequence. Consequently, the learned representations of the proteins, taken as the average of the embeddings of each amino acid (Fig. S1), must successfully extract generic features of the biological language of proteins. These features can then be used to fine-tune the model on downstream tasks.

In this work, the transformer model from (Rives et al, 2021) (esm1_t34_670M_UR100) was used, which was trained using the aforementioned procedure on the UniRef100 dataset (Suzek et al., 2007), containing >277M representative sequences. The pre-trained model was then fine-tuned every month on all the spike protein sequences registered in the GISAID data bank at the training date.

Gradient descent is used to minimize the loss function. We relied on the Adam optimizer (Kingma and Ba, 2014) and used a learning rate schedule. Batch size is 1. The fine-tuning started with a warm-up period of 100 mini-batches where the learning rate increased linearly from 10⁻⁷ to 10⁻⁵. After the warm-up period, the learning rate decreased following 10⁶\sqrt{k} where k represents the number of mini-batches.

### Inference and ML Scores Calculations

Once fine-tuned, the model can be used to compute the semantic change and the log-likelihood to characterize a Spike protein sequence.

Formally, an input sequence is represented by a sequence of tokens defined as x=(x₁, . . . , xₙ) where n is the number of tokens and ∀i∈[1, n], xᵢ∈χ where χ is a finite alphabet that contains the amino-acids and other tokens such as class and mask tokens. In this work, a class token is appended to all sequences before feeding them to the network, as such x₁ represents the class token, while x₂, . . . , xₙ represents the amino-acids, or masked amino-acids, in the spike protein sequence. The sequence x is passed through attention layers. The vector z (z₁, . . . , zₙ) is the output of the last attention layer where zᵢ is the sequence embedding vector at position i.

In this example, embedding vector a is a function of all input tokens (xⱼ)_{j∈[1,n]}. In opposition, in Bi-LSTM architectures, such as in (Hie et al., 2021), zᵢ would be a function of all inputs tokens except the one at the position i, (xⱼ)_{j∈[1,n]j≠i}.

In order to represent a protein sequence through a single embedding vector, which size does not depend on the protein sequence length, the following vector

$$\overline{z} = \frac{1}{n - 1} {\sum }_{j = 2}^{n} {z}_{i}$$

is defined as the embedding vector of the variant represented by sequence x. Note that the summation starts at the second position to ensure that the class token's embedding, which is at the first position, does not contribute to the sequence embedding.

The embedding of the Wuhan strain z̅_{wumuan} and the embedding of the D614G variant z̅_{D614G} are computed once for all. In this example, the semantic change of a variant x is computed as:

$$Δ \overline{z} = ‖ \overline{z} - {\overline{z}}_{wuhuan} ‖ + {‖ \overline{z} - {\overline{z}}_{D 614 G} ‖}_{1} ,$$

where ∥⋅∥₁ is the L1 norm.

The last attention layer output z is transformed by a feed-forward layer and a softmax activation into a vector of probabilities over tokens at each positions p=(p₁, . . . , pₙ) where P is a vector of probabilities at position i, pᵢ=(p(xᵢ=x₁|x), . . . , p(xᵢ=xₙ|x)).

The log-likelihood of a variant l(x) is computed from these probabilities. It is calculated as the sum of the log probabilities over all the positions of the Spike protein amino acids. Formally,

$$l ( x ) = {\sum }_{i = 2}^{n} log p ( {x}_{i} = {x}_{i} \vert x ) .$$

This quantity measures the likelihood of observing a variant sequence x according to the model. Therefore, the more sequences in the training data that are similar to a considered variant, the higher the log-likelihood of this variant will be. The proposed log-likelihood metric supports substitution, insertion, and deletion without the requirement of a reference.

### Implementation Details

The method may be implemented using the Pytorch (Paszke et al., 2019) deep learning framework. Model training and inferences may be performed on a high performance computing infrastructure. In such examples, the average training and inference time is <4 GPU days and <12 GPU hours, respectively, using Nvidia A100-SXM4-40 GB GPUs.

### Epitope Score

The epitope score attempts to capture the impact of mutations in the variant in question on recognition by experimentally assessed antibodies. To compute this score, the number of unique epitopes involving altered positions is enumerated, as measured across all the known antibody-spike complex structures deposited in Protein Data Bank.

This score, by design, may emphasize the effect of mutations on highly antigenic sites, such as the receptor-binding domain (RBD). This allows to approximate the expected weight of mutations, and to ascribe importance to non-RBD mutations, if and only if sufficient escape potential with regard to RBD-targeting antibodies is achieved.

### ACE2 Binding Score

279 receptor-binding domain (RBD) differentiated variants, including the wide type, were selected for in-silico simulation. For each variant, a putative structure was generated from which at least 500 structures were generated through a conformational sampling algorithm. These structures were further optimized with a probabilistic optimization algorithm, a variant of simulated annealing, aiming to overcome local energy barriers and follow a kinetically accessible path toward an attainable deep energy minimum with respect to a knowledge-based, protein-oriented potential. This results in 214,142 structures in total. For each structure, the change of binding energy when the interface forming chains are separated, versus when they are complexed was calculated. These measurements were aggregated per RBD variant using medians. Each metric is normalized by the metric on wild type, corresponding to no mutation on RBDs, such that the metrics for the wild type are all ones. Sequences having other RBD mutation combinations, representing very rare RBDs, corresponding to <9% of all known sequences, were excluded from this analysis, due to reasons of computational efficiency.

### Growth Score

The growth score is computed from the GISAID metadata. At a given date, only sequences that have been submitted within the last eight weeks are considered. For each lineage, its proportion among all submissions was calculated for the eight-week window and for the last week, denoted by r_{win} and rₗₐₛₜ correspondingly. The growth of the lineage is defined by their ratio, rₗₐₛₜ/r_{win}, measuring the change of the proportion. Having values larger than one means that the lineage is rising and smaller than one indicates declining.

### Scores Scaling and Merging

The semantic change, log-likelihood, epitope score, ACE2 binding score, and growth all have different scales and units. Thus, they cannot be compared directly. To make comparisons possible, a scaling strategy is introduced. For a given metric m, all the variants considered are ranked according to this metric. In the ranking system used, the higher rank the better. Variants with the same value for metric m will get the same rank. The ranks are then transformed into values between 0 and 100 through a linear projection to obtain the values for the scaled metric. All reported scores, except for log-likelihood, have been scaled according to this strategy.

Log-likelihood was observed to strongly penalize variants with a large number of mutations. An increased number of mutations may strongly affect fitness, thus explaining decreased log-likelihood. However, as the variants that are scored by EWS have been registered, which implies that they managed to infect hosts and replicate sufficiently to be detected, it is expected that they have at least minimal fitness. A variant with two mutations, whose log-likelihood is in the bottom 20^{th} percentile globally, is less likely to survive the evolutionary competition. A variant, with analogous log-likelihood, but with twenty mutations is more likely among others, similarly mutated ones. Thus, a group-based ranking strategy is introduced where each variant is ranked only among variants with a similar number of mutations. For each variant, having N mutations, its conditional log-likelihood score is ranked among all variants having at least M mutations, with M=min(max(0, N-10), 50). Deletions at N-terminal or C-terminal are considered as one single mutation for grouping. In each group, as for the other ranking technique, the ranks are then transformed into values between 0 and 100 through a linear projection to obtain the values for the scaled metric. Although this example uses all the samples that have no less than ten mutations fewer than the query, results are largely robust to the choice of a threshold.

The immune escape score is computed as the average of the scaled semantic change and of the scaled epitope score. The fitness prior score is computed as the average of the scaled conditional log-likelihood, the scaled ACE2 binding score, and the scaled growth.

### Pareto Score

Pareto optimality was defined over a set of lineages. Lineages are Pareto optimal within that set if there are no lineages in the set with both higher immune escape and higher infectivity scores. The Pareto score is a measure of the degree of Pareto optimality. Lineages with the highest Pareto score are Pareto optimal. Lineages with the second-best Pareto score would be Pareto optimal, if the Pareto optimal lineages were removed from the set, and so on.

To compute the Pareto score, first compute all the Pareto fronts that exist in the considered set of lineages. The first Pareto front corresponds to the set of lineages for which there does not exist any other lineage with both higher immune escape and infectivity score. The second Pareto front is computed as the Pareto front over the set of lineages remaining when removing the ones from the first Pareto front. Successive Pareto fronts are computed until all the lineages are assigned to a front. Then, a linear projection is used so that the lineages from the first front obtain a Pareto score of 100 and the ones from the last front get a Pareto score of 0.

### Semantic Change vs Epitope Score

Semantic change is a measure of how different the variant in question is with regard to the underlying statistical model (large ML model fine-tuned on spike protein sequences observed until a given time point). This value depends on the observations. Epitope score is a measure of how many distinct epitopes are evaded by the variant in question in comparison to wild type. This score, on the other hand, is computed purely based on known binding sites of antibodies, as reported in Protein Data Bank. It too changes with time with new discoveries of anti-spike antibodies, but to a lesser extent and is expected to converge to a stable value.

These scores, while intuitively correlated, are not collinear. Most of HRVs regarded as immune escaping (and denoted as VoCs, VoIs etc.) exhibit high semantic change, but are rather diverse in terms of Epitope Score (Fig. S7).

### Detecting HRVs by Standard ML Methods

A comparison is made between the detection capability of EWS compared to standard ML methods to highlight both the difficulty of the task and the need for deep learning representations.

For all the baselines protein sequences are represented by a vector of N binary components. To compute the representation for a protein sequence S deposited at time t, the N most prevalent mutations are calculated in all deposited sequences up to time t, inclusive. Each binary component of the representation equals 1 if the mutation is present in S and 0 otherwise. N=1280 is used for all the baselines, to permit a direct comparison with the methods proposed in this example.

As this example learns from unlabelled data, the unsupervised learning baseline: Uniform Manifold Approximation and Projection (UMAP) is considered first. UMAP is performed each week over the representations of all sequences available up to this week. A metric equivalent to the semantic change is computed as a mean L1 distance between the sequence projection and the projections of the Wuhan and D614G strains in UMAP spaces. The same detection technique as performed by EWS in this example is then used to flag every week a set of 20 variants suspected to be dangerous. Using UMAP only 5 out of 13 variants were detected with an average lead time of −43 days, see Table S.6. In comparison, analogous techniques applied in EWS example detect 12 out of 13 variants (8 ahead of time), with a mean lead time of 6 days. Both results suggest the benefit of more involved representations, such as the ones learned by Transformers models, especially in tasks like this one, where significance of the novel findings is difficult to approximate a priori.

Second, a supervised learning baseline was considered. Each week, all protein sequences that have been registered are labelled by 1 if it has been named as HRV any time before or during the week considered and 0 otherwise. A Generalized Linear Model (GLM) is built over the same 1280-dimensional representations of the sequences. The probability of belonging to the HRV class returned by the GLM is then used to rank sequences. Subsequently, the same detection technique performed by EWS is used to flag every week a set of 20 variants suspected to be dangerous. Using the GLM, 12 of 13 variants are eventually detected, with only 4 over 13 being detected before WHO naming, with an average lead time of 0.3 days, see Table S.6. The GLM approach is found to be less performing and less generic than the one proposed in this example. This makes the GLM approach less useful for pandemics that would attract less worldwide attention and thus have less or no labelled data available. In addition, the GLM approach is difficult or impossible to use early in a pandemic as there are no labels available and hallmark mutations are unlikely to be among the most common ones in population early on.

TABLE S.1
N = 310 nAbs Resolved 3D Structures PDB Identifiers.

| Code | Accession Date | Code | Accession Date | Code | Accession Date | Code | Accession Date | Code | Accession Date |
|---|---|---|---|---|---|---|---|---|---|
| 6W41 | 2020 Mar. 9 | 7MMO | 2021 Apr. 30 | 7KXJ | 2020 Dec. 4 | 7E5Y | 2021 Feb. 21 | 7CH4 | 2020 Jul. 5 |
| 6WPS | 2020 Apr. 27 | 7MY2 | 2021 May 20 | 7KXK | 2020 Dec. 4 | 7E7X | 2021 Feb. 28 | 7CH5 | 2020 Jul. 5 |
| 6WPT | 2020 Apr. 27 | 7MY3 | 2021 May 20 | 7KZB | 2020 Dec. 10 | 7E7Y | 2021 Feb. 28 | 7CHB | 2020 Jul. 5 |
| 6XC2 | 2020 Jun. 8 | 7N0G | 2021 May 25 | 7L02 | 2020 Dec. 10 | 7E86 | 2021 Mar. 1 | 7CHC | 2020 Jul. 5 |
| 6XC3 | 2020 Jun. 8 | 7N0H | 2021 May 25 | 7L06 | 2020 Dec. 11 | 7E88 | 2021 Mar. 1 | 7CHE | 2020 Jul. 5 |
| 6XC4 | 2020 Jun. 8 | 7N0I | 2021 May 25 | 7L09 | 2020 Dec. 11 | 7E8C | 2021 Mar. 1 | 7CHF | 2020 Jul. 5 |
| 6XC7 | 2020 Jun. 8 | 7N0R | 2021 May 25 | 7L0N | 2020 Dec. 11 | 7E8F | 2021 Mar. 1 | 7CHH | 2020 Jul. 5 |
| 6XCM | 2020 Jun. 8 | 7N3C | 2021 May 31 | 7L2C | 2020 Dec. 16 | 7E8M | 2021 Mar. 2 | 7CHO | 2020 Jul. 6 |
| 6XCN | 2020 Jun. 8 | 7N3D | 2021 May 31 | 7L2D | 2020 Dec. 16 | 7EAM | 2021 Mar. 7 | 7CHP | 2020 Jul. 6 |
| 6XDG | 2020 Jun. 10 | 7N3I | 2021 Jun. 1 | 7L2E | 2020 Dec. 16 | 7EAN | 2021 Mar. 7 | 7CHS | 2020 Jul. 6 |
| 6XE1 | 2020 Jun. 11 | 7N62 | 2021 Jun. 7 | 7L2F | 2020 Dec. 16 | 7EJ4 | 2021 Apr. 1 | 7CJF | 2020 Jul. 10 |
| 6XEY | 2020 Jun. 14 | 7N64 | 2021 Jun. 7 | 7L3N | 2020 Dec. 18 | 7EJ5 | 2021 Apr. 1 | 7CM4 | 2020 Jul. 24 |
| 6XKP | 2020 Jun. 26 | 7N8H | 2021 Jun. 14 | 7L56 | 2020 Dec. 21 | 7F62 | 2021 Jun. 24 | 7CR5 | 2020 Aug. 12 |
| 6XKQ | 2020 Jun. 26 | 7N8I | 2021 Jun. 14 | 7L57 | 2020 Dec. 21 | 7F63 | 2021 Jun. 24 | 7CWL | 2020 Aug. 29 |
| 6YLA | 2020 Apr. 6 | 7N9A | 2021 Jun. 17 | 7L58 | 2020 Dec. 21 | 7JMO | 2020 Aug. 2 | 7CWM | 2020 Aug. 29 |
| 6YM0 | 2020 Apr. 7 | 7N9B | 2021 Jun. 17 | 7L5B | 2020 Dec. 21 | 7JMP | 2020 Aug. 2 | 7CWN | 2020 Aug. 29 |
| 6YZ5 | 2020 May 6 | 7N9C | 2021 Jun. 17 | 7LAA | 2021 Jan. 6 | 7JMW | 2020 Aug. 3 | 7CWO | 2020 Aug. 29 |
| 6YZ7 | 2020 May 6 | 7N9E | 2021 Jun. 17 | 7LAB | 2021 Jan. 6 | 7JV2 | 2020 Aug. 20 | 7CWS | 2020 Aug. 31 |
| 6Z2M | 2020 May 17 | 7N9T | 2021 Jun. 18 | 7LCN | 2021 Jan. 11 | 7JV4 | 2020 Aug. 20 | 7CWT | 2020 Aug. 31 |
| 6Z43 | 2020 May 22 | 7ND4 | 2021 Jan. 30 | 7LD1 | 2021 Jan. 12 | 7JV6 | 2020 Aug. 20 | 7CWU | 2020 Aug. 31 |
| 6ZBP | 2020 Jun. 8 | 7ND5 | 2021 Jan. 30 | 7LDJ | 2021 Jan. 13 | 7JVA | 2020 Aug. 20 | 7CYH | 2020 Sep. 3 |
| 6ZCZ | 2020 Jun. 12 | 7ND6 | 2021 Jan. 30 | 7LJR | 2021 Jan. 30 | 7JVB | 2020 Aug. 20 | 7CYP | 2020 Sep. 4 |
| 6ZDG | 2020 Jun. 14 | 7ND7 | 2021 Jan. 30 | 7LM8 | 2021 Feb. 5 | 7JVC | 2020 Aug. 20 | 7CZP | 2020 Sep. 9 |
| 6ZDH | 2020 Jun. 14 | 7ND8 | 2021 Jan. 30 | 7LOP | 2021 Feb. 10 | 7JW0 | 2020 Aug. 24 | 7CZQ | 2020 Sep. 9 |
| 6ZER | 2020 Jun. 16 | 7ND9 | 2021 Jan. 30 | 7LQV | 2021 Feb. 15 | 7JX3 | 2020 Aug. 26 | 7CZR | 2020 Sep. 9 |
| 6ZFO | 2020 Jun. 17 | 7NDA | 2021 Jan. 30 | 7LQW | 2021 Feb. 15 | 7K43 | 2020 Sep. 14 | 7CZS | 2020 Sep. 9 |
| 6ZH9 | 2020 Jun. 21 | 7NDB | 2021 Jan. 30 | 7LRS | 2021 Feb. 17 | 7K45 | 2020 Sep. 14 | 7CZT | 2020 Sep. 9 |
| 6ZHD | 2020 Jun. 22 | 7NDC | 2021 Jan. 30 | 7LRT | 2021 Feb. 17 | 7K4N | 2020 Sep. 15 | 7CZU | 2020 Sep. 9 |
| 6ZLR | 2020 Jul. 1 | 7NDD | 2021 Jan. 30 | 7LS9 | 2021 Feb. 17 | 7K8M | 2020 Sep. 27 | 7CZV | 2020 Sep. 9 |
| 6ZXN | 2020 Jul. 30 | 7NEG | 2021 Feb. 4 | 7LSS | 2021 Feb. 18 | 7K8S | 2020 Sep. 27 | 7CZW | 2020 Sep. 9 |
| 7A25 | 2020 Aug. 16 | 7NEH | 2021 Feb. 4 | 7LX5 | 2021 Mar. 3 | 7K8T | 2020 Sep. 27 | 7CZX | 2020 Sep. 9 |
| 7A29 | 2020 Aug. 16 | 7NKT | 2021 Feb. 18 | 7LXW | 2021 Mar. 5 | 7K8U | 2020 Sep. 27 | 7CZY | 2020 Sep. 9 |
| 7A5R | 2020 Aug. 21 | 7NTC | 2021 Mar. 9 | 7LXX | 2021 Mar. 5 | 7K8V | 2020 Sep. 27 | 7CZZ | 2020 Sep. 9 |
| 7A5S | 2020 Aug. 21 | 7NX6 | 2021 Mar. 17 | 7LXY | 2021 Mar. 5 | 7K8W | 2020 Sep. 27 | 7D00 | 2020 Sep. 9 |
| 7AKD | 2020 Sep. 30 | 7NX7 | 2021 Mar. 17 | 7LXZ | 2021 Mar. 5 | 7K8X | 2020 Sep. 27 | 7D03 | 2020 Sep. 9 |
| 7B14 | 2020 Nov. 23 | 7NX8 | 2021 Mar. 17 | 7LY0 | 2021 Mar. 5 | 7K8Y | 2020 Sep. 27 | 7D0B | 2020 Sep. 9 |
| 7B17 | 2020 Nov. 23 | 7NX9 | 2021 Mar. 17 | 7LY2 | 2021 Mar. 5 | 7K8Z | 2020 Sep. 27 | 7D0C | 2020 Sep. 9 |
| 7B18 | 2020 Nov. 24 | 7NXA | 2021 Mar. 17 | 7LY3 | 2021 Mar. 5 | 7K90 | 2020 Sep. 27 | 7D0D | 2020 Sep. 9 |
| 7B27 | 2020 Nov. 26 | 7NXB | 2021 Mar. 17 | 7M3I | 2021 Mar. 18 | 7K9Z | 2020 Sep. 29 | 7D2Z | 2020 Sep. 17 |
| 7B3O | 2020 Dec. 1 | 7OAN | 2021 Apr. 19 | 7M42 | 2021 Mar. 19 | 7KFV | 2020 Oct. 15 | 7D30 | 2020 Sep. 17 |
| 7BEH | 2020 Dec. 23 | 7OAO | 2021 Apr. 19 | 7M6D | 2021 Mar. 25 | 7KFW | 2020 Oct. 15 | 7D4G | 2020 Sep. 23 |
| 7BEI | 2020 Dec. 23 | 7OAP | 2021 Apr. 19 | 7M6E | 2021 Mar. 25 | 7KFX | 2020 Oct. 15 | 7DCC | 2020 Oct. 24 |
| 7BEJ | 2020 Dec. 23 | 7OAQ | 2021 Apr. 20 | 7M6F | 2021 Mar. 25 | 7KFY | 2020 Oct. 15 | 7DCX | 2020 Oct. 27 |
| 7BEK | 2020 Dec. 23 | 7OAU | 2021 Apr. 20 | 7M6G | 2021 Mar. 25 | 7KGJ | 2020 Oct. 16 | 7DD2 | 2020 Oct. 27 |
| 7BEL | 2020 Dec. 23 | 7OAY | 2021 Apr. 20 | 7M6H | 2021 Mar. 25 | 7KGK | 2020 Oct. 16 | 7DD8 | 2020 Oct. 28 |
| 7BEM | 2020 Dec. 24 | 7OLZ | 2021 May 20 | 7M6I | 2021 Mar. 25 | 7KKK | 2020 Oct. 27 | 7DEO | 2020 Nov. 4 |
| 7BEN | 2020 Dec. 24 | 7OR9 | 2021 Jun. 4 | 7M71 | 2021 Mar. 26 | 7KKL | 2020 Oct. 27 | 7DET | 2020 Nov. 5 |
| 7BEO | 2020 Dec. 24 | 7ORA | 2021 Jun. 4 | 7M7B | 2021 Mar. 27 | 7KLG | 2020 Oct. 30 | 7DEU | 2020 Nov. 5 |
| 7BEP | 2020 Dec. 24 | 7ORB | 2021 Jun. 4 | 7M7W | 2021 Mar. 29 | 7KLH | 2020 Oct. 30 | 7DJZ | 2020 Nov. 22 |
| 7BWJ | 2020 Apr. 14 | 7P77 | 2021 Jul. 19 | 7M8J | 2021 Mar. 29 | 7KLW | 2020 Nov. 1 | 7DK0 | 2020 Nov. 22 |
| 7BYR | 2020 Apr. 24 | 7P78 | 2021 Jul. 19 | 7MDW | 2021 Apr. 6 | 7KM5 | 2020 Nov. 2 | 7DK4 | 2020 Nov. 23 |
| 7BZ5 | 2020 Apr. 26 | 7P79 | 2021 Jul. 19 | 7ME7 | 2021 Apr. 6 | 7KMG | 2020 Nov. 2 | 7DK5 | 2020 Nov. 23 |
| 7C01 | 2020 Apr. 29 | 7P7A | 2021 Jul. 19 | 7MEJ | 2021 Apr. 6 | 7KMH | 2020 Nov. 2 | 7DK6 | 2020 Nov. 23 |
| 7C2L | 2020 May 8 | 7R6W | 2021 Jun. 23 | 7MF1 | 2021 Apr. 8 | 7KMI | 2020 Nov. 2 | 7DK7 | 2020 Nov. 23 |
| 7C8V | 2020 Jun. 3 | 7R6X | 2021 Jun. 23 | 7MFU | 2021 Apr. 11 | 7KMK | 2020 Nov. 3 | 7DPM | 2020 Dec. 20 |
| 7C8W | 2020 Jun. 3 | 7R7N | 2021 Jun. 25 | 7MJI | 2021 Apr. 20 | 7KML | 2020 Nov. 3 | 7DZX | 2021 Jan. 26 |
| 7CAC | 2020 Jun. 8 | 7R8L | 2021 Jun. 26 | 7MJJ | 2021 Apr. 20 | 7KN5 | 2020 Nov. 4 | 7DZY | 2021 Jan. 26 |
| 7CAH | 2020 Jun. 8 | 7R8M | 2021 Jun. 26 | 7MJK | 2021 Apr. 20 | 7KN6 | 2020 Nov. 4 | 7E23 | 2021 Feb. 4 |
| 7CAI | 2020 Jun. 8 | 7R8N | 2021 Jun. 26 | 7MJL | 2021 Apr. 20 | 7KN7 | 2020 Nov. 4 | 7KSG | 2020 Nov. 22 |
| 7CAK | 2020 Jun. 8 | 7R8O | 2021 Jun. 26 | 7MKL | 2021 Apr. 24 | 7KQB | 2020 Nov. 14 | 7MM0 | 2021 Apr. 29 |
| 7CAN | 2020 Jun. 9 | 7R98 | 2021 Jun. 28 | 7MKM | 2021 Apr. 24 | 7KQE | 2020 Nov. 15 | 7RAL | 2021 Jul. 1 |
| 7CDI | 2020 Jun. 19 | 7RA8 | 2021 Jun. 30 | 7MLZ | 2021 Apr. 29 | 7KS9 | 2020 Nov. 21 | 7CDJ | 2020 Jun. 19 |

TABLE S.2
EWS Scores. EWS has in total five sub-scores grouped into immune escape and infectivity scores.
Each sub-score is normalized ranks that range between 0 and 100%. The average of the sub-scores
in each score category is computed to define immune escape and infectivity scores.

| Score Name | Score Category | Description as it relates to Example 2 |
|---|---|---|
| Epitope | Immune Escape | Measures the alteration of the spike protein at epitope positions by |
| | | counting the number of antibodies potentially escaped. |
| Semantic Change | Immune Escape | Measures the functional change of the spike protein using |
| | | Transformer-derived embedding differences with respect to the |
| | | wild type and D614G. |
| ACE2 Binding | Infectivity | Approximates the binding affinity using binding energies estimated |
| | | from in-silico simulations. |
| Conditional log- | Infectivity | Measures the relative rank of the measured existence probability of |
| Likelihood | | the spike protein using Transformer-derived log-likelihoods with |
| | | reference to other sequences with similar mutation count. |
| Growth | Infectivity | Calculated lineage-level growth using deposited sequence |
| | | metadata (retrospective data). |

TABLE S.3
Spike mutations in SARS-CoV-2 spike pseudoviruses and observed reduction
of neutralizing antibody response in pseudovirus neutralization assay.

| Lineage | WHO Nomenclature | Mutation | Reduction |
|---|---|---|---|
| B.1.1.7 | Alpha | H69- V70- Y144- N501Y A570D D614G P681H T716I S982A D1118H | 22.92% |
| B.1.1.7 + E484K | Alpha | H69- V70- Y144- E484K N501Y A570D D614G P681H T716I S982A D1118H | 74.65% |
| B.1.351 | Beta | L18F D80A D215G L242- A243- L244- R246I K417N E484K N501Y D614G A701V | 80.04% |
| B.1.351* | Beta | D80A D215G L242H K417N E484K N501Y D614G A701V | 47.19% |
| B.1.351** | Beta | D80A D215G L242- A243- L244- K417N E484K N501Y D614G A701V | 77.45% |
| P.1 | Gamma | L18F T20N P26S D138Y R190S K417T E484K N501Y H655Y T1027I V1176F | 47.66% |
| B.1.617.2 | Delta | T19R G142D E156G F157- R158- K417N L452R T478K D614G P681R D950N | 49.43% |
| AY.1 | Delta | T19R T95I G142D E156G F157- R158- W258L K417N L452R T478K K558N D614G | 48.09% |
| | | P681R D950N | |
| B.1.427/B.1.429 | Epsilon | S13I W152C L452R D614G | 52.57% |
| B.1.526 | Iota | L5F T95I D253G E484K D614G A701V | 10.94% |
| B.1.617.1 | Kappa | L452R E484Q D614G P681R | 18.34% |
| C.37 | Lambda | G75V T76I R246- S247- Y248- L249-T250- P251- G252- D253N L452Q F490S D614G | 34.22% |
| | | T859N | |
| C.37* | Lambda | G75V T76I L452Q F490S D614G T859N | 16.88% |
| B.1.1.529 | Omicron | A67V H69- V70- T95I G142D V143- Y144- Y145- N211I L212V ins214EPE G339D | 97.52% |
| | | S371L S373P S375F K417N N440K G446S S477N T478K E484A Q493R G496S Q498R | |
| | | N501Y Y505H T547K D614G H655Y N679K P681H N764K D796Y N856K Q954H | |
| | | N969K L981F | |
| A.VOI.V2 | | D80Y Y144- 1210- D215G R246- S247- Y248- L249M W258L R346K T478R E484K | 64.54% |
| | | H655Y P681H Q957H | |
| B.1.1.298 | | Y453F D614G I692V M1229I | 5.42% |
| B.1.160 | | S477N S494P D614G K1191N | 3.81% |
| B.1.258 | | H69- V70- L189F N439K D614G V772I | −9.28% |
| B.1.517 | | G181V G252V N501T D614G P812L | −7.87% |

TABLE S.4
Early detection of variants of concerns. The summary table shows that the EWS can detect WHO designated variants
months before the WHO official designation date. The average lead time for early detection across is 58 days.

| | | EWS | WHO | First | Lead | Number of registered | Number of registered |
|---|---|---|---|---|---|---|---|
| | | Detection | Designation | Submission | Time | sequences upon WHO | sequences upon EWS |
| WHO Label | Lineage | Date | Date | Date | (Days) | Detection | Designation |
| Alpha | B.1.1.7 | 2020 Oct. 14 | 2020 Dec. 18 | 2020 Oct. 14 | 65 | 1881 | 1 |
| Beta | B.1.351 | 2020 Nov. 27 | 2020 Dec. 18 | 2020 Nov. 26 | 21 | 218 | 15 |
| Gamma | P.1 | 2021 Jan. 10 | 2021 Jan. 11 | 2021 Jan. 10 | 1 | 29 | 4 |
| Delta | B.1.617.2 | | 2021 Apr. 4 | 2021 Mar. 23 | | 5 | |
| Epsilon | B.1.429 | 2020 Nov. 21 | 2021 Mar. 5 | 2020 Nov. 21 | 104 | 9957 | 1 |
| Zeta | P.2 | 2020 Dec. 4 | 2021 Mar. 17 | 2020 Dec. 2 | 103 | 1450 | 3 |
| Eta | B.1.525 | 2021 Jan. 4 | 2021 Apr. 4 | 2021 Jan. 4 | 90 | 1377 | 6 |
| Theta | P.3 | 2021 Feb. 25 | 2021 Mar. 24 | 2021 Feb. 25 | 27 | 102 | 1 |
| Iota | B.1.526 | 2021 Jan. 4 | 2021 Mar. 24 | 2020 Dec. 9 | 79 | 1120 | 2 |
| Kappa | B.1.617.1 | 2021 Mar. 31 | 2021 Apr. 4 | 2021 Mar. 5 | 4 | 167 | 167 |
| Lambda | C.37 | 2021 Feb. 26 | 2021 May 14 | 2021 Feb. 26 | 77 | 719 | 4 |
| Mu | B.1.621 | 2021 Apr. 30 | 2021 Aug. 30 | 2021 Mar. 11 | 122 | 3671 | 88 |
| Omicron | B.1.1.529 | 2021 Nov. 23 | 2021 Nov. 26 | 2021 Nov. 23 | 3 | 11 | 10 |

TABLE S.5
Welch's t-test p-values. Every week, all registered variants
are scored with the Pareto score. Welch's t-tests are
conducted to assess if respectively WHO designated variants
and VOCs can be separated from others. p-values are reported
every week between January 2020 and November 2021.

Welch t-test p-values

| Week | WHO Designated Variants | VOC |
|---|---|---|
| 2020 Dec. 13 | 8E−118 | 8E−118 |
| 2020 Dec. 20 | 7E−07 | 7E−07 |
| 2020 Dec. 27 | 5E−128 | 5E−128 |
| 2021 Jan. 3 | 7E−06 | 7E−06 |
| 2021 Jan. 10 | 5E−140 | 5E−140 |
| 2021 Jan. 17 | 2E−07 | 2E−07 |
| 2021 Jan. 24 | 4E−06 | 4E−06 |
| 2021 Jan. 31 | 5E−144 | 5E−144 |
| 2021 Feb. 7 | 2E−06 | 2E−06 |
| 2021 Feb. 14 | 6E−28 | 4E−19 |
| 2021 Feb. 21 | 7E−21 | 9E−08 |
| 2021 Feb. 28 | 1E−16 | 2E−08 |
| 2021 Mar. 7 | 8E−07 | 2E−19 |
| 2021 Mar. 14 | 2E−05 | 2E−07 |
| 2021 Mar. 21 | 1E−05 | 1E−20 |
| 2021 Mar. 28 | 1E−05 | 1E−22 |
| 2021 Apr. 4 | 4E−06 | 2E−19 |
| 2021 Apr. 11 | 3E−05 | 4E−18 |
| 2021 Apr. 18 | 5E−06 | 2E−20 |
| 2021 Apr. 25 | 6E−06 | 4E−08 |
| 2021 May 2 | 2E−12 | 5E−07 |
| 2021 May 9 | 3E−08 | 5E−06 |
| 2021 May 16 | 4E−09 | 8E−06 |
| 2021 May 23 | 1E−06 | 4E−04 |
| 2021 May 30 | 6E−07 | 4E−04 |
| 2021 Jun. 6 | 2E−04 | 2E−04 |
| 2021 Jun. 13 | 4E−05 | 2E−05 |
| 2021 Jun. 20 | 8E−06 | 2E−04 |
| 2021 Jun. 27 | 3E−05 | 3E−05 |
| 2021 Jul. 4 | 1E−04 | 2E−03 |
| 2021 Jul. 11 | 1E−05 | 1E−05 |
| 2021 Jul. 18 | 1E−04 | 3E−04 |
| 2021 Jul. 25 | 1E−03 | 1E−07 |
| 2021 Aug. 1 | 2E−04 | 6E−09 |
| 2021 Aug. 8 | 7E−06 | 5E−09 |
| 2021 Aug. 15 | 2E−05 | 9E−06 |
| 2021 Aug. 22 | 4E−07 | 3E−21 |
| 2021 Aug. 29 | 3E−05 | 4E−05 |
| 2021 Sep. 5 | 1E−04 | 5E−04 |
| 2021 Sep. 12 | 5E−05 | 1E−04 |
| 2021 Sep. 19 | 1E−04 | 2E−04 |
| 2021 Sep. 26 | 2E−06 | 9E−06 |
| 2021 Oct. 3 | 1E−04 | 1E−05 |
| 2021 Oct. 10 | 2E−06 | 7E−06 |
| 2021 Oct. 17 | 1E−07 | 4E−07 |
| 2021 Oct. 24 | 7E−07 | 3E−08 |
| 2021 Oct. 31 | 2E−05 | 2E−05 |
| 2021 Nov. 7 | 2E−04 | 3E−04 |
| 2021 Nov. 14 | 4E−03 | 3E−04 |
| 2021 Nov. 21 | 5E−05 | 7E−06 |

TABLE S.6
Comparison between EWS detection capabilities and three
baselines. Two baselines are based on unsupervised learning
(UMAP) and one baseline is supervised (GLM).

| | | | Number Days | |
|---|---|---|---|---|
| | Number | Number | Ahead EWS | Number |
| | Days Ahead | Days Ahead | Semantic | Days Ahead |
| HRV | UMAP | GLM | Change only | EWS |
| Alpha | −47 | −38 | 65 | 65 |
| Beta | — | −46 | 21 | 21 |
| Gamma | — | 1 | −121 | 1 |
| Delta | −58 | −25 | −78 | — |
| Epsilon | 64 | −27 | 66 | 104 |
| Zeta | — | 75 | — | 103 |
| Eta | — | 90 | 90 | 90 |
| Theta | — | −68 | 13 | 27 |
| Iota | −96 | 79 | −49 | 79 |
| Kappa | — | −25 | −29 | 4 |
| Lambda | −77 | −5 | 77 | 77 |
| Mu | — | −8 | 12 | 122 |
| Omicron | — | — | 3 | 3 |

### REFERENCES

Barnes, C. O., Jette, C. A., Abernathy, M. E., Dam, K.-M. A., Esswein, S. R., Gristick, H. B., Malyutin, A. G., Sharaf, N. G., Huey-Tubman, K. E., Lee, Y. E., et al. (2020). SARS-CoV-2 neutralizing antibody structures inform therapeutic strategies. Nature 588, 682-687.
Becht, E., McInnes, L., Healy, J., Dutertre, C.-A., Kwok, I. W. H., Ng, L. G., Ginhoux, F., and Newell, E. W. (2018). Dimensionality reduction for visualizing single-cell data using UMAP. Nat. Biotechnol. 37, 38-44.
Berger Rentsch, M., and Zimmer, G. (2011). A vesicular stomatitis virus replicon-based bioassay for the rapid and sensitive determination of multi-species type I interferon. PLoS ONE 6, e25858.
Corey, L., Beyrer, C., Cohen, M. S., Michael, N. L., Bedford, T., and Rolland, M. (2021). SARS-CoV-2 Variants in Patients with Immunosuppression. N. Engl. J Med. 385, 562-566.
Dejnirattisai, W., Zhou, D., Ginn, H. M., Duyvesteyn, H. M. E., Supasa, P., Case, J. B., Zhao, Y., Walter, T. S., Mentzer, A. J., Liu, C., et al. (2021). The antigenic anatomy of SARS-CoV-2 receptor binding domain. Cell 184, 2183-2200.e22.
Devlin, J., Chang, M.-W., Lee, K., and Toutanova, K. (2018). Bert: Pre-training of deep bidirectional transformers for language understanding. ArXiv Preprint ArXiv:1810.04805.
Elnaggar, A., Heinzinger, M., Dallago, C., Rihawi, G., Wang, Y., Jones, L., Gibbs, T., Feher, T., Angerer, C., Steinegger, M., et al. (2020). ProtTrans: towards cracking the language of Life's code through self-supervised deep learning and high performance computing. ArXiv Preprint ArXiv:2007.06225.
Hatcher, E. L., Zhdanov, S. A., Bao, Y., Blinkova, O., Nawrocki, E. P., Ostapchuck, Y., Schaffer, A. A., and Brister, J. R. (2017). Virus Variation Resource—improved response to emergent viral outbreaks. Nucleic Acids Res. 45, D482-D490.
Hie, B., Zhong, E. D., Berger, B., and Bryson, B. (2021). Learning the language of viral evolution and escape. Science 371, 284-288.
Ju, B., Zhang, Q., Ge, J., Wang, R., Sun, J., Ge, X., Yu, J., Shan, S., Zhou, B., Song, S., et al. (2020). Human neutralizing antibodies elicited by SARS-CoV-2 infection. Nature 584, 115-119.
Kingma, D. P., and Ba, J. (2014). Adam: A method for stochastic optimization. ArXiv Preprint ArXiv:1412.6980.
Liu, J., Liu, Y., Xia, H., Zou, J., Weaver, S. C., Swanson, K. A., Cai, H., Cutler, M., Cooper, D., Muik, A., et al. (2021a). BNT162b2-elicited neutralization of B.1.617 and other SARS-CoV-2 variants. Nature 596, 273-275.
Liu, Y., Liu, J., Xia, H., Zhang, X., Fontes-Garfias, C. R., Swanson, K. A., Cai, H., Sarkar, R., Chen, W., Cutler, M., et al. (2021b). Neutralizing Activity of BNT162b2-Elicited Serum. N. Engl. J Med. 384, 1466-1468.
Meier, J., Rao, R., Verkuil, R., Liu, J., Sercu, T., and Rives, A. (2021). Language models enable zero-shot prediction of the effects of mutations on protein function. BioRxiv.
Muik, A., Wallisch, A.-K., Sdnger, B., Swanson, K. A., Mühl, J., Chen, W., Cai, H., Maurus, D., Sarkar, R., Tureci, O., et al. (2021). Neutralization of SARS-CoV-2 lineage B.1.1.7 pseudovirus by BNT162b2 vaccine-elicited human sera. Science 371, 1152-1153.
O'Toole, Á., Scher, E., Underwood, A., Jackson, B., Hill, V., McCrone, J. T., Colquhoun, R., Ruis, C., Abu-Dahab, K., Taylor, B., et al. (2021). Assignment of epidemiological lineages in an emerging pandemic using the pangolin tool. Virus Evol. 7, veab064.
Paszke, A., Gross, S., Massa, F., Lerer, A., Bradbury, J., Chanan, G., Killeen, T., Lin, Z., Gimelshein, N., Antiga, L., et al. (2019). PyTorch: An Imperative Style, High-Performance Deep Learning Library. In Advances in Neural Information Processing Systems 32, (eds. H. Wallach, H. Larochelle, A. Beygelzimer, F. dAlché-Buc, E. Fox, and R. Garnett) (Curran Associates, Inc.), pp. 8024-8035.
Rambaut, A., Holmes, E. C., O'Toole, Á., Hill, V., McCrone, J. T., Ruis, C., du Plessis, L., and Pybus, O. G. (2020). A dynamic nomenclature proposal for SARS-CoV-2 lineages to assist genomic epidemiology. Nat. Microbiol. 5, 1403-1407.
Rives, A., Meier, J., Sercu, T., Goyal, S., Lin, Z., Liu, J., Guo, D., Ott, M., Zitnick, C. L., Ma, J., et al. (2021). Biological structure and function emerge from scaling unsupervised learning to 250 million protein sequences. Proc Natl Acad Sci USA 118.
Sahin, U., Muik, A., Vogler, I., Derhovanessian, E., Kranz, L. M., Vormehr, M., Quandt, J., Bidmon, N., Ulges, A., Baum, A., et al. (2021). BNT162b2 vaccine induces neutralizing antibodies and poly-specific T cells in humans. Nature 595, 572-577.
Shu, Y., and McCauley, J. (2017). GISAID: Global initiative on sharing all influenza data—from vision to reality. Eurosurveillance 22, 30494.
Singh, J., Rahman, S. A., Ehtesham, N. Z., Hira, S., and Hasnain, S. E. (2021). SARS-CoV-2 variants of concern are emerging in India. Nat. Med. 27, 1131-1133.
Steinegger, M., Meier, M., Mirdita, M., Vöhringer, H., Haunsberger, S. J., and Söding, J. (2019). HH-suite3 for fast remote homology detection and deep protein annotation. BMC Bioinformatics 20, 473.
Suzek, B. E., Huang, H., McGarvey, P., Mazumder, R., and Wu, C. H. (2007). UniRef comprehensive and non-redundant UniProt reference clusters. Bioinformatics 23, 1282-1288.
Tanaka, S., Nelson, G., Olson, C. A., Buzko, O., Higashide, W., Shin, A., Gonzalez, M., Taft, J., Patel, R., Buta, S., et al. (2021). An ACE2 Triple Decoy that neutralizes SARS-CoV-2 shows enhanced affinity for virus variants. Sci. Rep. 11, 12740.
The Technical Advisory Group on SARS-CoV-2 Virus Evolution (TAG-VE) (2021). Classification of Omicron (B.1.1.529): SARS-CoV-2 Variant of Concern.
Twohig, K. A., Nyberg, T., Zaidi, A., Thelwall, S., Sinnathamby, M. A., Aliabadi, S., Seaman, S. R., Harris, R. J., Hope, R., Lopez-Bernal, J., et al. (2021). Hospital admission and emergency care attendance risk for SARS-CoV-2 delta (B.1.617.2) compared with alpha (B.1.1.7) variants of concern: a cohort study. Lancet Infect. Dis.
UniProt Consortium (2019). UniProt: a worldwide hub of protein knowledge. Nucleic Acids Res. 47, D506-D515.
Vaswani, A., Shazeer, N., Parmar, N., Uszkoreit, J., Jones, L., Gomez, A. N., Kaiser, U., and Polosukhin, I. (2017). Attention is all you need. In Advances in Neural Information Processing Systems, pp. 5998-6008.
Weigang, S., Fuchs, J., Zimmer, G., Schnepf, D., Kern, L., Beer, J., Luxenburger, H., Ankerhold, J., Falcone, V., Kemming, J., et al. (2021). Within-host evolution of SARS-CoV-2 in an immunosuppressed COVID-19 patient as a source of immune escape variants. Nat. Commun. 12, 6405.
Weisblum, Y., Schmidt, F., Zhang, F., DaSilva, J., Poston, D., Lorenzi, J. C., Muecksch, F., Rutkowska, M., Hoffmann, H.-H., Michailidis, E., et al. (2020). Escape from neutralizing antibodies by SARS-CoV-2 spike protein variants. ELife 9.
Yan, R., Wang, R., Ju, B., Yu, J., Zhang, Y., Liu, N., Wang, J., Zhang, Q., Chen, P., Zhou, B., et al. (2021). Structural basis for bivalent binding and inhibition of SARS-CoV-2 infection by human potent neutralizing antibodies. Cell Res. 31, 517-525.
Zhang, L., Mann, M., Syed, Z. A., Reynolds, H. M., Tian, E., Samara, N. L., Zeldin, D. C., Tabak, L. A., and Ten Hagen, K. G. (2021). Furin cleavage of the SARS-CoV-2 spike is modulated by O-glycosylation. Proc Natl Acad Sci USA 118.

1. A method of identifying a number of variants of concern of a reference disease associated immunogen, the method comprising:
using, by one or more processing units of a computing infrastructure, a language model to perform inference on data representing each of a plurality of variants and data representing the reference immunogen;
determining, by the processing unit(s), for each of the plurality of variants and the reference immunogen, a characteristic vector derived from an output feature map of a hidden layer of the language model;
for each of the plurality of variants, generating, by the processing unit(s), a measure of distance for the variant that includes calculating a measure of distance between the characteristic vector of the variant and the characteristic vector of the reference immunogen;
generating, by the processing unit(s), a semantic change score for each variant based on the generated measure of distance for that variant; and
selecting, by the processing unit(s), a variant of the reference immunogen based, at least in part, on the generated the semantic change scores.

2. The method claim 1, wherein the step of generating a semantic change score comprises ranking the plurality of variants by their respective generated measures of distance and then transforming the ranked values into scaled semantic change scores.

3. The method claim 1, further comprising:
identifying, by the processing unit(s), one or more epitope regions of the reference immunogen and identifying, by the processing unit(s), each corresponding range of positions associated with each epitope region in the data representing the reference immunogen;
generating, by the processing unit(s), an epitope value for each variant based on locations of mutations in that variant relative to the reference immunogen.

4. The method of claim 3, further comprising:
generating, by the processing unit(s), an epitope score for each variant from the epitope values by ranking the plurality of variants by the respective epitope value; and
transforming, by the processing unit(s), the ranked values into a scaled epitope score.

5. The method of claim 2, comprising calculating, by the processing unit(s), an immune escape score that is a combination of the semantic change score and the epitope value.

6. The method of claim 1, comprising calculating, by the processing unit(s), for each variant, a likelihood value for the data representing the variant.

7. The method of claim 6, wherein:
the data representing the variant is data describing a protein sequence of the variant, and
the likelihood value for the data representing the variant is derived from a likelihood for each amino acid in the data describing the protein sequence.

8. The method of claim 7, comprising generating, by the processing unit(s), a likelihood score for each variant from the likelihood values by ranking the plurality of variants by their respective likelihood value.

9. The method of claim 1, comprising determining, by the processing unit(s), a binding value that represents an estimated binding energy between a variant and a corresponding human receptor.

10. The method of claim 9, wherein determining the binding value includes:
simulating, by the processing unit(s), one or more structures associated with the variant, and
estimating, by the processing unit(s), using the simulated one or more structures, a change of binding energy when an interface of the structure is bound with a model of a human receptor.

11. The method of claim 10, comprising:
generating, by the processing unit(s), a binding value that is the estimated change in binding energy, and
calculating, by the processing unit(s), a binding score for each variant from the binding values by ranking the plurality of variants by their respective binding value.

12. The method of claim 1, comprising determining, by the processing unit(s), a growth value for each variant, wherein the growth value is a measure of growth of submissions associated with each variant within a dataset.

13. The method of claim 8, further comprising calculating, by the processing unit(s), an infectivity score that is a combination of the likelihood score, the binding score, and the growth value.

14. The method of claim 5, further comprising calculating, by the processing unit(s), a Pareto score which is determined by calculating a Pareto front using the immune escape score and the infectivity score.

15. The method of claim 14, wherein calculating the Pareto score comprises:
calculating, by the processing unit(s), a first Pareto front corresponding to a set of variants for which there does not exist any other variant with both higher immune escape and infectivity score;
calculating, by the processing unit(s), successive Pareto fronts obtained from the set of variants remaining after removing the first or succeeding Pareto fronts;
calculating, by the processing units, successive Pareto fronts until all variants are assigned to a front;
assigning, by the processing unit(s), each variant to a Pareto value depending on the number of the front that they were member of; and
obtaining, by the processing unit(s), a Pareto score by transforming the Pareto values to a scaled Pareto score.

16. The method of claim 1, wherein the step of generating a measure of distance for the variant comprises calculating a measure of distance between the characteristic vector of the variant and the characteristic vector of the reference immunogen and calculating a measure of distance between the characteristic vector of the variant and the characteristic of one or more additional reference immunogens.

17. A method for use in association with therapeutic or vaccine development, the method comprising:
using, by one or more processing unit(s) of a computing infrastructure, a language model to perform inference on data representing each of a plurality of variants and data representing reference disease associated immunogen;
determining, by the processing unit(s), for each of the plurality of variants and the reference immunogen, a characteristic vector derived from an output feature map of a hidden layer of the language model;
for each of the plurality of variants, generating, by the processing unit(s), a measure of distance for the variant that includes calculating a measure of distance between the characteristic vector of the variant and the characteristic vector of the reference immunogen;
generating, by the processing unit(s), a semantic change score for each variant based on the generated measure of distance for that variant; and
selecting, by the processing unit(s), a variant of the reference immunogen based, at least in part, on the generated the semantic change scores.

18. A method of performing a trend analysis on the prevalence of variants of concern of a reference immunogen in a subject or a population, the method comprising:
using, by one or more processing unit(s) of a computing infrastructure, a language model to perform inference on data representing each of a plurality of variants and data representing the reference immunogen;
determining, by the processing unit(s), for each of the plurality of variants and the reference immunogen, a characteristic vector derived from an output feature map of a hidden layer of the language model;
for each of the plurality of variants, generating, by the processing unit(s), a measure of distance for the variant that includes calculating a measure of distance between the characteristic vector of the variant and the characteristic vector of the reference immunogen; and
generating, by the processing unit(s), a semantic change score for each variant based on the generated measure of distance for that variant; selecting a variant of the reference immunogen based, at least in part, on the generated the semantic change scores.

19. The method of claim 5, comprising selecting, by the processing unit(s), the variant of the reference immunogen based, at least in part, on the calculated immune escape score.

20. The method of claim 1, comprising identifying, by the processing unit(s), clusters of variants, based on the characteristic vector extracted for each of the plurality of variants.

21. The method of claim 3, comprising:
assigning, by the processing unit(s), different weightings to the different epitope regions, wherein the weightings correspond to a measure of confidence of the existence of the epitope; and
generating, by the processing unit(s), the epitope value for each variant based on the locations of mutations in that variant relative to the reference immunogen and the assigned weightings.
